SEQUENCE LISTING (53 sequences)

SEQ ID NO: 1 - protein, 613 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 2 - protein, 612 aa, Artificial Sequence
  Amino acid sequence of EPA with L552V/deltaE553 detoxifying mutation
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 3 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 4 - protein, 535 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 5 - protein, 535 aa, Artificial Sequence
  CRM197, non-toxic mutant of diphtheria toxin
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 6 - protein, 162 aa, Pseudomonas aeruginosa
MAVDMFIKIGDVKGESKDKTHAEEIDVLAWSWGMSQSGSMHMGGGGGAGKVNVQDLSFTKYIDKSTPNLMMACSSGKHYPQAKLTIRKAGGENQVEYLIITLKEVLVSSVSTGGSGGEDRLTENVTLNFAQVQVDYQPQKADGAKDGGPIKYGWNIRQNVQA

SEQ ID NO: 7 - protein, 221 aa, Escherichia coli
GIFSRFADIVNANINALLEKAEDPQKLVRLMIQEMEDTLVEVRSTSARALAEKKQLTRRIEQASAREVEWQEKAELALLKEREDLARAALIEKQKLTDLIKSLEHEVTLVDDTLARMKKEIGELENKLSETRARQQALMLRHQAANSSRDVRRQLDSGKLDEAMARFESFERRIDQMEAEAESHSFGKQKSLDDQFAELKADDAISEQLAQLKAKMKQDNQ

SEQ ID NO: 8 - protein, 396 aa, Escherichia coli
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 9 - protein, 445 aa, Neisseria gonorrhoeae
MIPQYEQPKVEVAETFQNDTSVSSIRAVDLGWHDYFADPRLQKLIDIALERNTSLRTAVLNSEIYRKQYMIERNNLLPTLAANANGSRQGSLSGGNVSSSYNVGLGAASYELDLFGRVRSSSEAALQGYFASVANRDAAHLSLIATVAKAYFNERYAEEAMSLAQRVLKTREETYNAVRIAVQGRRDFRRRPAPAEALIESAKADYAHAARSREQARNALATLINRPIPEDLPAGLPLDKQFFVEKLPAGLSSEVLLDRPDIRAAEHALKQANANIGAARAAFFPSIRLTGSVGTGSVELGGLFKSGTGVWAFAPSITLPIFTWGTNKANLDVAKLRQQAQIVAYESAVQSAFQDVANALAAREQLDKAYDALSKQSRASKEALRLVGLRYKHGVSGALDLLDAERSSYSAEGAALSAQLTRAENLADLYKALGGGLKRDTQTGK

SEQ ID NO: 10 - protein, 522 aa, Staphylococcus aureus
ASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTFNDTNTVSSVNSPQNSTNAENVSTTQDTSTEATPSNNESAPQSTDASNKDVVNQAVNTSAPRMRAFSLAAVAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPED

SEQ ID NO: 11 - protein, 340 aa, Staphylococcus aureus
VAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPED

SEQ ID NO: 12 - protein, 340 aa, Artificial Sequence
  ClfAN2N3P116S/Y118A
VAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMSAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPED

SEQ ID NO: 13 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 14 - protein, 293 aa, Artificial Sequence
  Amino acid sequence of mature HlaH35L
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 15 - protein, 293 aa, Artificial Sequence
  Amino acid sequence of mature Hla H35L/H48C/G122C
ADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 16 - protein, 258 aa, Artificial Sequence
  HlaPSGS
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTPSGSVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 17 - protein, 5 aa, Artificial Sequence
  consensus sequence
  MISC_FEATURE (1)..(1): Xaa can be Asp or Glu
  MISC_FEATURE (2)..(2): Xaa is any amino acid apart from proline
  MISC_FEATURE (4)..(4): Xaa is any amino acid apart from proline
  MISC_FEATURE (5)..(5): Xaa can be Ser or Thr
XXNXX

SEQ ID NO: 18 - protein, 7 aa, Artificial Sequence
  consensus sequence
  MISC_FEATURE (2)..(2): Xaa can be Asp or Glu
  MISC_FEATURE (3)..(3): Xaa is any amino acid apart from proline
  MISC_FEATURE (5)..(5): Xaa is any amino acid apart from proline
  MISC_FEATURE (6)..(6): Xaa can be Ser or Thr
KXXNXXK

SEQ ID NO: 19 - protein, 9 aa, Artificial Sequence
  consensus sequence
  MISC_FEATURE (1)..(1): Xaa can be Lys or Arg
  MISC_FEATURE (2)..(2): Xaa can be Asp or Glu
  MISC_FEATURE (3)..(3): Xaa is any amino acid apart from proline
  MISC_FEATURE (5)..(5): Xaa is any amino acid apart from proline
  MISC_FEATURE (6)..(6): Xaa can be Ser or Thr
  MISC_FEATURE (7)..(7): Xaa is any amino acid except lysine or arginine
  MISC_FEATURE (8)..(8): Xaa is any amino acid except lysine or arginine
  MISC_FEATURE (9)..(9): Xaa can be Lys or Arg
XXXNXXXXX

SEQ ID NO: 20 - protein, 9 aa, Artificial Sequence
  consensus sequence
  MISC_FEATURE (2)..(2): Xaa can be Asp or Glu
  MISC_FEATURE (3)..(3): Xaa is any amino acid apart from proline
  MISC_FEATURE (5)..(5): Xaa is any amino acid apart from proline
  MISC_FEATURE (6)..(6): Xaa can be Ser or Thr
KXXNXXSAR

SEQ ID NO: 21 - protein, 19 aa, Escherichia coli
MIKFLSALILLLVTTAAQA

SEQ ID NO: 22 - protein, 21 aa, Escherichia coli
MKKTAIAIAVALAGFATVAQA

SEQ ID NO: 23 - protein, 26 aa, Escherichia coli
MKIKTGARILALSALTTMMFSASALA

SEQ ID NO: 24 - protein, 22 aa, Erwinia carotovora
MKYLLPTAAAGLLLLAAQPAMA

SEQ ID NO: 25 - protein, 23 aa, Escherichia coli
MSFKKIIKAFVIMAALVSVQAHA

SEQ ID NO: 26 - protein, 28 aa, Escherichia coli
MFKFKKKFLVGLTAAFMSISMFSATASA

SEQ ID NO: 27 - protein, 19 aa, Escherichia coli
MKKIWLALAGLVLAFSASA

SEQ ID NO: 28 - protein, 21 aa, Escherichia coli
MKQALRVAFGFLILWASVLHA

SEQ ID NO: 29 - protein, 26 aa, Artificial Sequence
  SipA signal sequence
MKMNKKVLLTSTMAASLLSVASVQAS

SEQ ID NO: 30 - protein, 325 aa, Artificial Sequence
  Amino acid sequence of Hla H35L/H48C/G122C with N-terminal S, Flgl signal sequence, C-terminal GSHRHR, and KDQNRTK substitution for residue K131
MIKFLSALILLLVTTAAQASADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDQNRTKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTNGSHRHR

SEQ ID NO: 31 - protein, 327 aa, Artificial Sequence
  Amino acid sequence of mature Hla H35L/G122C/H48C with N-terminal S, Flgl signal sequence, C-terminal GSHRHR and KDSNITSAR substitution for residue K131
MIKFLSALILLLVTTAAQASADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDSNITSARIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTNGSHRHR

SEQ ID NO: 32 - protein, 327 aa, Artificial Sequence
  Amino acid sequence of mature Hla H35L/G122C/H48C with N-terminal S, Flgl signal sequence, C-terminal GSHRHR and KDSNSTSAR substitution for residue K131
MIKFLSALILLLVTTAAQASADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDSNSTSARIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTNGSHRHR

SEQ ID NO: 33 - protein, 327 aa, Artificial Sequence
  Amino acid sequence of mature Hla H35L/G122C/H48C with N-terminal S, Flgl signal sequence, C-terminal GSHRHR and KDSNVTSAR substitution for residue K131,
MIKFLSALILLLVTTAAQASADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDSNVTSARIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTNGSHRHR

SEQ ID NO: 34 - protein, 327 aa, Artificial Sequence; identical to SEQ ID NO: 33
  Amino acid sequence of mature Hla H35L/G122C/H48C with N-terminal S, Flgl signal sequence, C-terminal GSHRHR and KDSNATSAR substitution for residue K131

SEQ ID NO: 35 - protein, 299 aa, Artificial Sequence
  Amino acid sequence of mature Hla H35L/H48C/G122C with KDQNRTK substitution for residue K131
ADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDQNRTKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 36 - protein, 301 aa, Artificial Sequence
  Amino acid sequence of mature Hla H35L/G122C/H48C with KDSNITSAR substitution for residue K131
ADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDSNITSARIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 37 - protein, 301 aa, Artificial Sequence
  Amino acid sequence of mature Hla H35L/G122C/H48C with KDSNSTSAR substitution for residue K131
ADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDSNSTSARIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 38 - protein, 301 aa, Artificial Sequence
  Amino acid sequence of mature Hla H35L/G122C/H48C with KDSNVTSAR substitution for residue K131
ADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDSNVTSARIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 39 - protein, 301 aa, Artificial Sequence; identical to SEQ ID NO: 38
  Amino acid sequence of mature Hla H35L/G122C/H48C with KDSNATSAR substitution for residue K131

SEQ ID NO: 40 - protein, 7 aa, Artificial Sequence
  KDQNRTK glycosite
KDQNRTK

SEQ ID NO: 41 - protein, 7 aa, Artificial Sequence
  KDQNATK glycosite
KDQNATK

SEQ ID NO: 42 - protein, 9 aa, Artificial Sequence
  KDSNITSAR glycosite
KDSNITSAR

SEQ ID NO: 43 - protein, 9 aa, Artificial Sequence
  KDSNSTSAR glycosite
KDSNSTSAR

SEQ ID NO: 44 - protein, 9 aa, Artificial Sequence
  KDSNVTSAR glycosite
KDSNVTSAR

SEQ ID NO: 45 - protein, 9 aa, Artificial Sequence
  KDSNATSAR glycosite
KDSNATSAR

SEQ ID NO: 46 - protein, 9 aa, Artificial Sequence
  consensus sequence
  MISC_FEATURE (1)..(1): Xaa can be Lys or Arg
  MISC_FEATURE (2)..(2): Zaa represent 0-9 amino acids which are any amino acid except cysteine, methionine, asparagine, glutamine, lysine or arginine.
  MISC_FEATURE (3)..(3): Xaa can be Asp or Glu
  MISC_FEATURE (4)..(4): Xaa is any amino acid except proline, lysine or arginine
  MISC_FEATURE (6)..(6): Xaa is any amino acid except proline, lysine or arginine
  MISC_FEATURE (7)..(7): Xaa can be Ser or Thr
  MISC_FEATURE (8)..(8): Zaa represent 0-9 amino acids which are any amino acid except cysteine, methionine, asparagine, glutamine, lysine or arginine.
  MISC_FEATURE (9)..(9): Xaa can be Lys or Arg
XXXXNXXXX

SEQ ID NO: 47 - protein, 9 aa, Artificial Sequence
  consensus sequence
  MISC_FEATURE (1)..(1): Xaa can be Lys or Arg
  MISC_FEATURE (2)..(2): Xaa represent 0-9 amino acids which are any amino acid except cysteine, methionine, asparagine, glutamine, lysine or arginine.
  MISC_FEATURE (3)..(3): Xaa can be Asp or Glu
  MISC_FEATURE (4)..(4): Xaa is any amino acid except proline, cysteine, methionine, asparagine, glutamine, lysine or arginine.
  MISC_FEATURE (6)..(6): Xaa is any amino acid except proline, cysteine, methionine, asparagine, glutamine, lysine or arginine.
  MISC_FEATURE (7)..(7): Xaa can be Ser or Thr
  MISC_FEATURE (8)..(8): Xaa represent 0-9 amino acids which are any amino acid except cysteine, methionine, asparagine, glutamine, lysine or arginine.
  MISC_FEATURE (9)..(9): Xaa can be Lys or Arg
XXXXNXXXX

SEQ ID NO: 48 - protein, 8 aa, Artificial sequence
  Peptide 42T-50K named PTP-2
TGDLVTYK

SEQ ID NO: 49 - protein, 10 aa, Artificial sequence
  Peptide 225A-234K named PTP-3
AADNFLDPNK

SEQ ID NO: 50 - protein, 5 aa, Artificial sequence
  Spacer
GSGGG

SEQ ID NO: 51 - protein, 351 aa, Artificial sequence
  Amino acid sequence of mature Hla H35L/G122C/H48C with KDSNITSAR glycosite substitution for residue K131; glycosite KDSNVTSAR at N-terminal with GSGGG spacers before and after this glycosite; Flgl signal sequence; and His tag at C-terminal
MIKFLSALILLLVTTAAQASAGSGGGKDSNVTSARGSGGGKLADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDSNITSARIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTNGSHHHHHH

SEQ ID NO: 52 - protein, 345 aa, Artificial sequence
  Amino acid sequence of mature Hla H35L/G122C/H48C with KDSNITSAR glycosite substitution for residue K131; glycosite KDSNVTSAR at C-terminal with GSGGG spacers before this glycosite; Flgl signal sequence; and His tag at C-terminal
MIKFLSALILLLVTTAAQASAADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDSNITSARIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTNLGSGGGKDSNVTSARGSHHHHHH

SEQ ID NO: 53 - protein, 357 aa, Artificial sequence
  Amino acid sequence of mature Hla H35L/G122C/H48C with KDSNITSAR glycosite at C-terminal end preceded by GSGGG spacers; glycosite KDSNVTSAR at N-terminal with GSGGG spacers before and after this glycosite; Flgl signal sequence; and His tag at C-terminal
MIKFLSALILLLVTTAAQASAGSGGGKDSNVTSARGSGGGKLADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTNLGSGGGKDSNITSARGSHHHHHH

(19) United States
(12) Patent Application Publication
(10) Pub. No.: US 2022/0257751 A1
(43) Pub. Date: Aug. 18, 2022

(54) QUANTIFICATION OF BIOCONJUGATE GLYCOSYLATION

(71) Applicant: GLAXOSMITHKLINE BIOLOGICALS SA, Rixensart (BE)

(72) Inventors: Massimilliano BIAGINI, Siena (IT); Lucia Eleonora FONTANA, Siena (IT); Anna GALEA, Siena (IT); Nathalie NORAIS, Siena (IT); Maria SCARSELLI, Siena (IT)

(73) Assignee: GLAXOSMITHKLINE BIOLOGICALS SA, Rixensart (BE)

(21) Appl. No.: 17/628,667

(22) PCT Filed: Jul. 21, 2020
(86) PCT No.: PCT/EP2020/070595

§ 371 Date: Jan. 20, 2022

(30) Foreign Application Priority Data
Jul. 23, 2019 (EP) 19187963.4

(51) Int. Cl. A61K 39/385; C07K 14/205; C07K 14/21; C07K 14/22; C07K 14/245; C07K 14/31; C07K 14/33; C12P 21/00; C12Q 1/37; G01N 33/68

(52) U.S. Cl. CPC A61K 39/385; C07K 14/205; C07K 14/21; C07K 14/22; C07K 14/245; A61K 2039/6037; C07K 14/33; C12P 21/005; C12Q 1/37; G01N 33/68; C07K 14/31

(57) ABSTRACT

The present invention provides analytical tools for the characterisation of bioconjugates, in particular for the measurement of glycosylation levels, and methods for absolute quantification of glycosylation sequences, as well as sequences and glycosylation sites for use in such methods.

### FIELD OF THE INVENTION

The present invention relates to analytical tools for the characterisation of bioconjugates, in particular the measurement of glycosylation levels.

### BACKGROUND TO THE INVENTION

Glycoconjugate vaccines have been proven to be efficacious and cost effective in the prevention of infectious diseases caused by encapsulated bacteria. In the last decade, new approaches have been taken for glycoconjugate vaccine production, including techniques exploiting bacterial N-glycosylation. The most well-developed of these ‘bioconjugation’ technologies is based on the production of glycoproteins in Escherichia coli in which the Campylobacter jejuni glycosylation machinery PglB is co-expressed with enzymes involved in a pathogen polysaccharide chain biosynthesis and a target carrier protein, acceptor of the polysaccharide (Wacker et al, 2002, Science 298:1790-3), which is engineered to contain a consensus sequence for PglB.

The main strength of the bioconjugation technology is the selectivity of the site of glycosylation on the carrier protein sequence. This is achieved by selectively introducing into the carrier protein sequence specific amino acids, creating one or more effective consensus sequences for selective conjugation to the polysaccharide chain. The core consensus sequence of PglB is D/E-X-N-Z-S/T wherein X and Z are independently any amino acid apart from proline (see Wacker et al, 2002, Science 298:1790-3), but an extended consensus sequence of K-D/E-X-N-Z-S/T-K is glycosylated with higher efficiency and is more widely used (see for example WO2019/121924 and WO2019/121926).

This technology is of particular interest for vaccine development especially when carrier proteins are selected to have a dual role as carrier and antigen, as it can preserve key protective epitopes. Furthermore, bioconjugation shows a higher suitability to large scale production in manufacturing of vaccines in comparison to chemical conjugation, as it decreases the need for pathogen handling, permits a reduction in production process steps, and is less time and resource-consuming.

Bioconjugate vaccine candidates have been recently proposed for the prevention of Gram-negative (Salmonella enterica, Shigella spp, pathogenic E. coli) and Gram-positive pathogen infections (Streptococcus pneumoniae and Staphylococcus aureus) (e.g. Wetter et al, 2013, Glycoconj J. 30:511-22. Engineering, conjugation, and immunogenicity assessment of Escherichia coli O121 O antigen for its potential use as a typhoid vaccine component; Wacker et al., 2014, J. Infect. Dis. 209:1551-1561; Van den Dobbelsteen et al., 2016, Vaccine 34:4152-60). Among them, S. aureus alpha toxin (Hla) bioconjugated with S. aureus type 5 CP (Hla-CP5) was shown to induce rabbit or mice protective antibodies recognizing both the glycan and the protein moieties, demonstrating the dual role of Hla protein as carrier and as protective antigen (Wacker et al., 2014, J. Infect. Dis. 209:1551-1561). This data is particularly relevant for the development of a vaccine preventing the diffusion of S. aureus, which is becoming challenging to fight due to the increase of the multi-drug resistant strains spreading around the world, including hospital and community-related infections strains.

Despite the increasing relevance of bioconjugates in vaccine development field, robust analytical tools needed to evaluate efficacy of carrier glycosylation are still lacking (Micoli, F. et al, 2018, Molecules 23:1451). In particular, precise quantification of the extent of glycosylation remains a challenging task, although this information is fundamental to fulfil potential regulatory requirements and to monitor antigen production and characterization. There is thus a need in the art for robust and reliable methods of accurately quantifying absolute levels of glycosylation site occupancy in bioconjugates.

### SUMMARY OF THE INVENTION

The inventors have designed universal consensus sequences for protein N-glycosylation which are suitable for the absolute quantification of glycosylation site occupancy. Specifically, the use of these consensus sequences allows the overall protein concentration and the unglycosylated portion of the protein to be quantified simultaneously by using heavy isotope-labeled internal standards in a liquid chromatography with tandem mass spectrometry (LC-MS/MS) analysis, and the extent of site occupancy to be accurately determined (Zhu et al 2015, J Am Soc Mass Spectrom. 25:1012-7).

The inventors devised a method based on that of Zhu et al for quantification of glycosylation using as a model a Hla carrier protein containing the glycosylation consensus site KDQNRTK of SEQ ID NO:40 (described in WO2019/121924). The strategy is based on the quantification of the natively unglycosylated form of the glycopeptide, using isotopically labeled internal standards. In brief, two sets of heavy isotope labeled peptide standards are spiked into the sample before trypsin digestion, and the digested sample is analyzed by LC-MS. One set of peptide standards is employed to determine the total glycoprotein amount, while the other standard monitors the unglycosylated amount of the glycoprotein. In this way, the abundance of the glycosylated portion of the protein is calculated by subtracting the unglycosylated protein amount from the total protein amount, and the site occupancy is then determined.

However, the KDQNRTK (SEQ ID NO:40) consensus sequence was found to generate a tryptic peptide which was too short and too hydrophilic to allow a LC-MS quantification. The same problem would be encountered for other commonly used consensus sequences such as KDQNATK (SEQ ID NO:41).

The inventors thus set out to design universal consensus sites that would be compatible with the method, i.e. which would be glycosylated with at least the same efficiency as the previously used sites and would also generate tryptic peptides detectable by LC-MS. Using Hla as a proof of principle, they were able to successfully design consensus sequences suitable for the quantification of the extent of conjugation by mass spectrometry.

The present invention permits the amount of unglycosylated carrier in the final product to be quantified; the rate of bioconjugation to be followed in-process; and the extent of glycosylation on single and multiple consensus sites to be quantified. Moreover, the selectivity of detection reduces the necessity for extensive sample purification supporting the characterisation of in-process and final product.

In a first aspect, therefore, the invention provides a consensus sequence comprising or consisting of the following amino acid sequence:

K/R-Z₀₋₉-D/E-X-N-Y-S/T-Z₀₋₉-K/R
wherein X and Y are independently any amino acid except proline, and Z represents any amino acid. In a preferred embodiment, X and Y are independently any amino acid except proline, lysine or arginine. In an embodiment, Z represents any amino acid except lysine or arginine. In an embodiment, X, Y and/or Z are not aromatic or hydrophobic amino acids. In a preferred embodiment, Z represents any amino acid except cysteine, methionine, asparagine, glutamine, lysine or arginine (eg SEQ ID NO: 47).

In a specific embodiment, the invention provides a consensus sequence(s) comprising or consisting of the amino acid sequence K/R-D/E-X₁-N-X₂-S/T-Z₁-Z₂-K/R (SEQ ID NO 19), wherein X₁ and X₂ are independently any amino acid apart from proline, lysine or arginine and wherein Z₁ and Z₂. are not lysine or arginine or cysteine. In an embodiment, the consensus sequence comprises or consists of the amino acid sequence of SEQ ID NO: 20. In an embodiment, the consensus sequence comprises or consists of the amino acid sequence of any one of SEQ ID No: 42, SEQ ID No: 43; SEQ ID No: 44 or SEQ ID No 45, preferably SEQ ID Nos: 42-44.

In one aspect, the invention provides a modified carrier protein, modified in that it comprises one or more consensus sequence(s) of the invention. Thus, the invention provides a modified carrier protein modified in that it comprises one or more consensus sequence(s) comprising or consisting of the amino acid sequence K/R-(Z)₀₋₉-D/E-X-N-Y-S/T-(Z)₀₋₉-K/R (SEQ ID NO: 46) as defined above; for example the amino acid sequence K/R-D/E-X₁-N-X₂-S/T-Z₁-Z₂-K/R (SEQ ID NO 19), wherein X₁ and X₂ are independently any amino acid apart from proline, lysine or arginine and wherein Z₁ and Z₂. are not lysine or arginine or cysteine. In an embodiment, the consensus sequence comprises or consists of the amino acid sequence of SEQ ID NO: 20. In an embodiment, the consensus sequence comprises or consists of the amino acid sequence of any one of SEQ ID No: 42, SEQ ID No: 43; SEQ ID No: 44 or SEQ ID No 45, preferably SEQ ID Nos: 42-44.

In an embodiment, said consensus sequence has been substituted for one or more amino acids of the carrier protein sequence. In another embodiment said consensus sequence has been inserted into the carrier protein sequence.

The modified carrier protein may comprise more than one said consensus sequence, optionally at least 2, 3, 4 or 5 consensus sequences. In a preferred embodiment, where a modified carrier protein contains more than one consensus sequence, all of said consensus sequences are different i.e. have different amino acid sequences.

The carrier protein may be any protein, preferably a protein able to elicit a T-dependent immune response. In specific embodiments, the carrier protein is CRM197, TT from Clostridium tetani, EPA from P. aeruginosa, Hcp1 from P. aeruginosa, Hla from S. aureus, ClfA from S. aureus, MBP from E., PspA from E. coli, or MtrE from N. gonorrhoeae.

In an embodiment, the carrier protein comprises or consists of an amino acid sequence of any one of SEQ ID Nos: 1 to 16 or an amino acid sequence at least 80%, 85%, 90%, 92%, 95%, 96%, 97%, 98% or 99% identical to any one of SEQ ID NOs. 1 to 16.

In an embodiment, the modified carrier protein comprises or consists of an amino acid sequence at least 80%, 85%, 90%, 92%, 95%, 96% or 97% identical to any one of SEQ ID NOs. 1 to 16.

The modified carrier protein may be glycosylated. The invention also provides a glycosylated carrier protein of the invention, and conjugates (e.g. bioconjugates) comprising a modified carrier protein of the invention linked to a polysaccharide. The polysaccharide is linked to an amino acid on the modified carrier protein selected from asparagine, aspartic acid, glutamic acid, lysine, cysteine, tyrosine, histidine, arginine or tryptophan (preferably asparagine). In an embodiment, the capsular polysaccharide is from the same organism as the carrier protein. In an embodiment, the capsular polysaccharide is from a different organism to the carrier protein.

In an embodiment, the polysaccharide is a bacterial capsular polysaccharide, for example Staphylococcus aureus type 5 capsular saccharide, Staphylococcus aureus type 8 capsular saccharide, N. meningitidis serogroup A capsular saccharide (MenA), N. meningitidis serogroup C capsular saccharide (MenC), N. meningitidis serogroup Y capsular saccharide (MenY), N. meningitidis serogroup W capsular saccharide (MenW), H. influenzae type b capsular saccharide (Hib), Group B Streptococcus group I capsular saccharide, Group B Streptococcus group II capsular saccharide, Group B Streptococcus group III capsular saccharide, Group B Streptococcus group IV capsular saccharide, Group B Streptococcus group V capsular saccharide, Vi saccharide from Salmonella typhi, N. meningitidis LPS (lipopolysaccharide, such as L3 and/or L2), M. catarrhalis LPS, H. influenzae LPS, Shigella O-antigens, P. aeruginosa O-antigens, E. coli O-antigens or S. pneumoniae a S. aureus capsular polysaccharide.

According to a further aspect of the invention, there is provided a polynucleotide encoding a modified carrier protein or bioconjugate of the invention.

According to a further aspect of the invention, there is provided a vector comprising a polynucleotide encoding a modified carrier protein or bioconjugate of the invention.

According to a further aspect of the invention, there is provided a host cell comprising:

i) one or more nucleic acids that encode glycosyltransferase(s);
ii) a nucleic acid that encodes an oligosaccharyl transferase;
iii) a nucleic acid that encodes a modified Carrier protein of the invention; and optionally
iv) a nucleic acid that encodes a polymerase (e.g. wzy).

The nucleic acid that encodes the modified carrier protein may be carried on a plasmid in the host cell, or may be integrated into the genome of the host cell. The host cell is preferably E. coli.

According to a further aspect of the invention, there is provided a process for producing a bioconjugate that comprises (or consists of) a modified carrier protein linked to a saccharide, said method comprising: (i) culturing a host cell of the invention under conditions suitable for the production of proteins and (ii) isolating the bioconjugate produced by said host cell. Also provided is a bioconjugate obtained or obtainable by said process, wherein said bioconjugate comprises a polysaccharide linked to a modified carrier protein.

According to a further aspect of the invention, there is provided an immunogenic composition comprising the modified carrier protein of the invention, or a conjugate of the invention, or a bioconjugate of the invention and a pharmaceutically acceptable excipient or carrier.

According to a further aspect of the invention, there is provided a method of making a immunogenic composition of the invention comprising the step of mixing a modified carrier protein or the conjugate or the bioconjugate of the invention with a pharmaceutically acceptable excipient or carrier.

According to a further aspect of the invention, there is provided an immunogenic composition comprising the modified carrier protein, conjugate or bioconjugate of the invention.

According to a further aspect of the invention, there is provided a method of making the immunogenic composition comprising the modified carrier protein, conjugate or bioconjugate of the invention comprising the step of mixing the modified carrier protein or the conjugate or the bioconjugate of the invention with a pharmaceutically acceptable excipient or carrier.

According to a further aspect of the invention, there is provided a vaccine comprising the immunogenic composition of the invention and a pharmaceutically acceptable excipient or carrier.

According to a further aspect of the invention, there is provided a method for the treatment or prevention of a bacterial infection in a subject in need thereof comprising administering to said subject a therapeutically effective amount of the modified carrier protein, conjugate or bioconjugate of the invention.

According to a further aspect of the invention, there is provided a method of immunising a human host against a bacterial infection comprising administering to the host an immunoprotective dose of the modified carrier protein, conjugate or bioconjugate of the invention.

According to a further aspect of the invention, there is provided a method of inducing an immune response to a bacterium in a subject, the method comprising administering a therapeutically or prophylactically effective amount of the modified carrier protein, conjugate or bioconjugate of the invention.

According to a further aspect of the invention, there is provided a modified carrier protein, conjugate or bioconjugate of the invention for use in the treatment or prevention of a disease caused by bacterial infection.

According to a further aspect of the invention, there is provided use of the modified carrier protein, conjugate or bioconjugate of the invention in the manufacture of a medicament for the treatment or prevention of a disease caused by bacterial infection.

In specific embodiments, said bacterium or bacterial infection is selected from the group consisting of Staphylococcus aureus, N. meningitidis, H. influenzae, H. influenzae type b, Group B Streptococcus, S. typhi, M. catarrhalis LPS, S. flexneri, P. aeruginosa, E. coli or S. pneumoniae.

According to a further aspect of the invention, there is provided a method of measuring the level of glycosylation site occupancy of a carrier protein of the invention, said method comprising digesting the glycosylated carrier protein with a protease, e.g. trypsin; subjecting the digested protein to LC-MS; determining the concentration U of unmodified carrier protein; determining the concentration T of total carrier protein; and calculating glycosylation site occupancy according to the following equation:

$$Site Occupancy ( \% ) = \frac{( Total - unmodified ) carrier concentration}{Total carrier concentration} \times 100$$

The concentration U of unmodified carrier protein is determined by determining the concentration of a peptide fragment corresponding to the consensus sequence of the invention. The concentration T of total carrier protein may suitably be determined by determining the concentration of one or more peptide fragments which are unique to said carrier protein.

### DESCRIPTION OF THE FIGURES

FIG. 1: Workflow of the strategy undertaken.

FIGS. 2A and 2B: In silico design of consensus sequences. (A) Statistical analysis of the occurrence of amino acids in the region from −6 to +6 of the glycosylated Asn residue found in 32 native C. jejuni glycoproteins. The analysis is reported in Kowarik et al. EMBO J. 2006; 25(9): 1957-66. The height of the box reflects the frequency of the amino acid residues in the naturally occurring consensus sequences. The Asn residue (in position 0, site of glycosylation) and the Asp and Thr residues in position −2 and +2 respectively, demonstrated as crucial for an efficient glycosylation, are reported in bold in grey boxes. The amino acid residues in position −3, −1, +1, +3 and +4, respectively, represented in bold in grey boxes, were selected for the design of the four consensus sequences (B).

FIGS. 3A and 3B: Efficacy of bioconjugation of the newly designed carriers assessed by Western blot. The periplasmic fractions prepared from E. coli engineered for the expression of Hla-i-CP5, Hla-v-CP5 and Hla-s-CP5 (lanes 1-3, FIG. 3A) were analyzed by Western blot using a rabbit anti-Hla-CP5 serum. The levels of expression were compared to the optimized Hla bearing the consensus sequence KDQNRTK which is not compatible with the MS analysis (lane 4, FIG. 3A). As negative control the Western blot analysis of the periplasmic fractions prepared from the respective strains that do not express Hla are reported (lanes 1-4, FIG. 3B). The positive signal observed might be related to the reaction intermediate undecaprenyl-linked CP5 molecules, produced and assembled during the process.

FIG. 4: Dose-response linearity. As an example the dose-response linearity curve of PTP-i is reported. To build up the calibration curve, on y axes are plotted the L/H area ratios responses determined by spiking in 50 μg of E. coli periplasmic fraction a fixed amount of heavy forms of PTPi (0.1 pmol/μg) and scalar concentration of light PTPi (ranging from 0.0125 to 1.6 pmol/μg, x axes), before the trypsin digestion. According to the International Conference on Harmonization (ICH) Guidelines (www.ich.org/products/guidelines/quality/article/quality-guidelines.html), the lower limit of quantification (LLOQ) for each peptide was set as the lowest concentration point on the fitted curve that can be quantitively detected and defined as 10 σ/S, where σ=the standard deviation of the response and S=the slope of the calibration curve and was calculated as 0.08 pmoles/ug of periplasmic proteins. Defined in an identical way, the LLOQ was 0.11 and 0.06 pmoles/ug of periplasmic proteins for PTP-s and PTP-v, respectively.

FIG. 5: (A): Schematic illustration of the constructs Hla-N, 131, Hla-N,C, and Hla-131,C each carrying two consensus sequences for bioconjugation to S. aureus CP5, alternatively located at N-terminal, C-terminal or at position 131 on the carrier protein, with their respective calculated extent of glycosylation in the CP5 bioconjugates. (B): Curve of the % of glycosylation of the consensus sequence inserted in position 131 as function of the total amount of the protein in the periplasmic fraction.

### DETAILED DESCRIPTION OF THE INVENTION

### Definitions

As used herein, the term “carrier protein” refers to a protein covalently attached to a polysaccharide antigen (e.g. saccharide antigen) to create a conjugate (e.g. bioconjugate). A carrier protein activates T-cell mediated immunity in relation to the polysaccharide antigen to which it is conjugated.

ClfA: clumping factor A from a staphylococcal bacterium, in particular S. aureus.
CRM197: non-toxic mutant of diphtheria toxin.
EPA: exotoxin A of Pseudomonas aeruginosa.
Hla: Haemolysin A, also known as alpha toxin, from a staphylococcal bacterium, in particular S. aureus.
Hcp1: Protein Hcp1 from Pseudomonas aeruginosa
MBP: Maltose/maltodextrin binding protein from Escherichia coli.
MtrE: Membrane Transporter E from Neisseria gonorrhoeae.
PspA, phage shock protein A from Escherichia coli.
CP: Capsular polysaccharide.

As used herein, the term “bioconjugate” refers to conjugate between a protein (e.g. a carrier protein) and an antigen (e.g. a saccharide) prepared in a host cell background, wherein host cell machinery links the antigen to the protein (e.g. N-links). Usually, in a bioconjugate the polysaccharide is linked to asparagine via N-acetylglucosamine.

As used herein, the term “glycosite” refers to an amino acid sequence recognized by a bacterial oligosaccharyltransferase, e.g. PglB of C. jejuni. The minimal consensus sequence for PglB is D/E-X-N-Z-S/T (SEQ ID NO: 17), while an extended consensus sequence K-D/E-X-N-Z-S/T-K (SEQ ID NO: 18) has also been defined. Exemplary and alternative glycosite sequences are described herein.

Any amino acid apart from proline (pro, P): refers to an amino acid selected from the group consisting of alanine (ala, A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), serine (ser, S), threonine (thr, T), tryptophan (trp, W), tyrosine (tyr, Y), valine (val, V).

As used herein, the term “effective amount,” in the context of administering a therapy (e.g. an immunogenic composition or vaccine of the invention) to a subject refers to the amount of a therapy which has a prophylactic and/or therapeutic effect(s).

As used herein, the term “subject” refers to an animal, in particular a mammal such as a primate (e.g. human).

As used herein, reference to a percentage sequence identity between two amino or nucleic acid sequences means that, when aligned, that percentage of amino acids or bases are the same in comparing the two sequences. This alignment and the percent homology or sequence identity can be determined using software programs known in the art, for example those described in section 7.7.18 of Current Protocols in Molecular Biology (F. M. Ausubel et al., eds., 1987, Supplement 30). A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2: 482-489. Percentage identity to any particular sequence (e.g. to a particular SEQ ID) is ideally calculated over the entire length of that sequence. The percentage sequence identity between two sequences of different lengths is preferably calculated over the length of the longer sequence. Global or local alignments may be used. Preferably, a global alignment is used.

As used herein, the term “purifying” or “purification” of a fusion protein or protein of interest, or conjugate (e.g. bioconjugate) thereof, means separating it from one or more contaminants. A contaminant is any material that is different from said fusion protein or protein of interest, or conjugate (e.g. bioconjugate) thereof. Contaminants may be, for example, cell debris, nucleic acid, lipids, proteins other than the fusion protein or protein of interest, polysaccharides and other cellular components.

A “recombinant” polypeptide is one which has been produced in a host cell which has been transformed or transfected with nucleic acid encoding the polypeptide or produces the polypeptide as a result of homologous recombination.

As used herein, the term “conservative amino acid substitution” involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a parental polypeptide.

As used herein, the term “deletion” is the removal of one or more amino acid residues from the protein sequence. Typically, no more than about from 1 to 6 residues (e.g. 1 to 4 residues) are deleted at any one site within the protein molecule.

As used herein, the term “insertion” is the addition of one or more non-native amino acid residues in the protein sequence. Typically, no more than about from 1 to 6 residues (e.g. 1 to 4 residues) are inserted at any one site within the protein molecule.

As used herein, the term ‘comprising’ indicates that other components in addition to those named may be present, whereas the term ‘consisting of’ indicates that other components are not present, or not present in detectable amounts. The term ‘comprising’ naturally includes the term ‘consisting of’.

### Carrier Proteins

Conjugation of T-independent antigens such as saccharides to carrier proteins has long been established as a way of enabling T-cell help to become part of the immune response for a normally T-independent antigen. In this way, an immune response can be enhanced by allowing the development of immune memory and boostability of the response. The carrier protein turns the T-independent saccharide antigen into a T-dependent antigen capable of triggering an immune memory response. Successful conjugate vaccines which have been developed by conjugating bacterial capsular saccharides to carrier proteins are known in the art; carrier proteins which have been widely used in commercialised vaccines include tetanus toxoid, diphtheria toxoid, CRM197 and protein D from Haemophilus influenzae. CRM197 is currently used in the Streptococcus pneumoniae capsular polysaccharide conjugate vaccine PREVENAR™ (Pfizer) and protein D, tetanus toxoid and diphtheria toxoid are currently used as carriers for capsular polysaccharides in the Streptococcus pneumoniae capsular polysaccharide conjugate vaccine SYNFLORIX™ (GlaxoSmithKline). Other carrier proteins known in the art include EPA (exotoxin A of P. aeruginosa) for Staphlyococcus aureus serotype 5 and 8 capsular polysaccharides (Wacker et al., 2014, J Infect. Dis. 209:1551-1561).

It is also possible to use as a carrier protein, a protein antigen from the same organism as the conjugated polysaccharide, in order to increase the protective capacity of the conjugate. For example, the S. aureus protein antigens Hla have successfully been used as a carrier protein for S. aureus capsular polysaccharide. Vaccination with Hla-CP5 and ClfA-CP8 bioconjugates was able to induce functional antibodies to both the capsular polysaccharide and protein antigens and confer protection from S. aureus infection in animal models, as described in in WO2019/121924, WO2019/121926 and PCT/EP2019/053463. Thus, any protein antigen could be a candidate for use as a carrier protein in a polysaccharide conjugate vaccine. Preferably, said protein antigen would be from the same organism as the polysaccharide. However, it would also be possible to use a protein antigen from a different organism, for example to confer protection against multiple pathogens.

Exemplary carrier proteins which may be used with the present invention are described below.

EPA: Exotoxin A of Pseudomonas aeruginosa.

In an embodiment, the carrier protein is exotoxin A from Pseudomonas aeruginosa (EPA). Said EPA may comprise the amino acid sequence of SEQ ID NO: 1 or an amino acid sequence at least 80%, 85%, 90%, 92%, 95%, 96%, 97%, 98% or 99% A identical to SEQ ID NO: 1.

Accordingly, there is provided in one aspect of the present invention, a modified EPA protein comprising an amino acid sequence of SEQ ID NO: 1 or an amino acid sequence at least 80%, 85%, 90%, 92%, 95%, 96%, 97%, 98% or 99% A identical to SEQ ID NO: 1, modified in that it comprises one or more consensus sequence(s) comprising or consisting of the amino acid sequence K/R-(Z)₀₋₉-D/E-X-N-Y-S/T-(Z)₀₋₉-K/R (SEQ ID NO: 46) as defined above; for example one or more consensus sequence(s) having the amino acid sequence K/R-D/E-X₁-N-X₂-S/T-Z₁-Z₂-K/R (SEQ ID NO 19), wherein X₁ and X₂ are independently any amino acid apart from proline, arginine and lysine, and wherein Z₁ and Z₂. are not lysine or arginine. In an embodiment, said consensus sequence comprises or consists of the amino acid sequence of SEQ ID NO: 20. In an embodiment, said consensus sequence comprises or consists of the amino acid sequence of any one of SEQ ID Nos: 42-45. In a preferred embodiment, said consensus sequence comprises or consists of the amino acid sequence of any one of SEQ ID Nos: 42-44.

The EPA protein may be further modified in that it comprises a detoxifying mutation, for example L to V substitution at the amino acid position corresponding to position L552 of SEQ ID NO: 1, and/or deletion of E553 of SEQ ID NO: 1, or at equivalent positions within an amino acid sequence at least 80%, 85%, 90%, 92%, 95%, 96%, 97%, 98% or 99% identical to SEQ ID NO: 1 (e.g. SEQ ID NO: 2); and/or one or more amino acids have been substituted by one or more consensus sequence(s) K/R-D/E-X₁-N-X₂-S/T-Z₁-Z₂-K/R (SEQ ID NO 19). In an embodiment, said substitution is substitution of A375, A376 or K240 of SEQ ID NO: 1. Hence, the protein of interest may comprise the amino acid sequence of SEQ ID NO: 2 or an amino acid sequence at least 80%, 85%, 90%, 92%, 95%, 96%, 97%, 98% or 99% identical to SEQ ID NO: 2, with insertion or substitution of one or more amino acids with a consensus sequence having an amino acid sequence of SEQ ID NO: 19, 20 or 42-47.

In an embodiment, said modified EPA protein comprises more than one said consensus sequence, for example 2, 3, 4 or 5 consensus sequences. In a preferred embodiment, wherein multiple consensus sequences are present, the consensus sequences have different sequences in order that glycosylation at each individual site may be distinguished.

In an embodiment, the modified EPA protein of the invention may be derived from an amino acid sequence at least 80%, 85%, 90%, 92%, 95%, 96%, 97%, 98% or 99% identical to SEQ ID NO: 1 which is an immunogenic fragment and/or a variant of SEQ ID NO: 1. In an embodiment, the modified EPA protein of the invention may be derived from an immunogenic fragment of SEQ ID NO: 1 or 2 comprising at least about 15, at least about 20, at least about 40, or at least about 60 contiguous amino acid residues of the full length sequence, wherein said polypeptide is capable of eliciting an immune response specific for said amino acid sequence.

In an embodiment, the modified EPA protein of the invention may be derived from an amino acid sequence at least 80%, 85%, 90%, 92%, 95%, 96%, 97%, 98% or 99% A identical to SEQ ID NO: 1 which is a variant of SEQ ID NO: 1 which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, the modified EPA protein of the present invention may be derived from a variant in which 1 to 10, 5 to 10, 1 to 5, 1 to 3, 1 to 2 or 1 amino acid are substituted, deleted, or added in any combination.

In an embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated on the basis of the method described by Jameson and Wolf (CABIOS 4:181-186 [1988]).

The term “modified EPA protein” refers to a EPA acid sequence (for example, having a EPA amino acid sequence of SEQ ID NO: 1 or an amino acid sequence at least 80%, 85%, 90%, 91%, 92%, 93%, 94%, 95%, 96%, 97%, 98% or 99% identical to SEQ ID NO: 1), which EPA amino acid sequence may be a wild-type mature EPA amino acid sequence (for example, a wild-type amino acid sequence of SEQ ID NO: 1), which has been modified by the addition, substitution or deletion of one or more amino acids (for example, addition (insertion) of a consensus sequence(s) with amino acid sequence SEQ ID NO:19 or 46; or by substitution of one or more amino acids by a consensus sequence(s) with amino acid sequence SEQ ID NO:19 or 46. The modified EPA protein may also comprise further modifications (additions, substitutions, deletions) as well as the addition or substitution of one or more consensus sequence(s). For example, a signal sequence and/or peptide tag may be added. Additional amino acids at the N and/or C-terminal may be included to aid in cloning (for example, after the signal sequence or before the peptide tag, where present). In an embodiment, the modified EPA protein of the invention may be a non-naturally occurring EPA protein.

In an embodiment of the invention, one or more amino acids (e.g. 1-7 amino acids, e.g. one amino acid) of the modified EPA amino acid sequence (for example, having an amino acid sequence of SEQ ID NO: 1 or a EPA amino acid sequence at least 80%, 85%, 90%, 91%, 92%, 93%, 94%, 95%, 96%, 97%, 98% or 99% identical to SEQ ID NO: 1, e.g. SEQ ID No 2) have been substituted by a consensus sequence(s) with amino acid sequence SEQ ID NO: 46 or SEQ ID NO: 19, for example SEQ ID NO 20 or 42-45 or 47, in particular SEQ ID Nos: 42-44. For example, a single amino acid in the EPA amino acid sequence (e.g. SEQ ID NO: 1) may be replaced with a said consensus sequence. Alternatively, 2, 3, 4, 5, 6 or 7 amino acids in the EPA amino acid sequence (e.g. SEQ ID NO: 1 or a EPA amino acid sequence at least 80%, 85%, 90%, 92%, 95%, 96%, 97%, 98% or 99% identical to SEQ ID NO: 1) may be replaced with said consensus sequence.

Introduction of a consensus sequence(s) enables the modified EPA protein to be glycosylated. Thus, the present invention also provides a modified EPA protein of the invention wherein the modified EPA protein is glycosylated. In specific embodiments, the consensus sequences are introduced into specific regions of the EPA amino acid sequence, e.g. surface structures of the protein, at the N or C termini of the protein, and/or in loops that are stabilized by disulfide bridges.

A person skilled in the art will understand that when the EPA amino acid sequence is a variant and/or fragment of an amino acid sequence of SEQ ID NO: 2, such as an amino acid sequence at least 80%, 85%, 90%, 92%, 95%, 96%, 97%, 98% or 99% A identical to SEQ ID NO: 2, the reference to “between amino acids . . . ” refers to the position that would be equivalent to the defined position, if this sequence was lined up with an amino acid sequence of SEQ ID NO: 1 in order to maximise the sequence identity between the two sequences Sequence alignment tools are described above. The addition or deletion of amino acids from the variant and/or fragment of SEQ ID NO: 1 could lead to a difference in the actual amino acid position of the consensus sequence in the mutated sequence, however, by lining the mutated sequence up with the reference sequence, the amino acid in in an equivalent position to the corresponding amino acid in the reference sequence can be identified and hence the appropriate position for addition or substitution of the consensus sequence can be established.

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein.

In an embodiment, the modified EPA protein of the invention further comprises a “peptide tag” or “tag”, i.e. a sequence of amino acids that allows for the isolation and/or identification of the modified EPA protein. For example, adding a tag to a modified EPA protein of the invention can be useful in the purification of that protein and, hence, the purification of conjugate vaccines comprising the tagged modified EPA protein. Exemplary tags that can be used herein include, without limitation, histidine (HIS) tags. I one embodiment, the tag is a hexa-histidine tag. In certain embodiments, the tags used herein are removable, e.g. removal by chemical agents or by enzymatic means, once they are no longer needed, e.g. after the protein has been purified. Optionally the peptide tag is located at the C-terminus of the amino acid sequence. Optionally the peptide tag comprises six histidine residues at the C-terminus of the amino acid sequence. The peptide tag may comprise or be preceded by one, two or more additional amino acid residues, for example alanine, serine and/or glycine residues, e.g. GS.

Hla: Haemolysin A of Staphylococcus aureus

In an embodiment, the carrier protein is Hla (haemolysin A of S. aureus, also known as alpha toxin). Hla has successfully been used as a carrier protein for S. aureus capsular polysaccharide, as described above. The mature wild-type amino acid sequence of Hla is given in SEQ ID NO 13.

Accordingly, there is provided in one aspect of the present invention, a modified Hla protein comprising an amino acid sequence of SEQ ID NO: 13 or an amino acid sequence at least 80%, 85%, 90%, 92%, 95%, 96%, 97%, 98% or 99% identical to SEQ ID NO: 13, modified in that it comprises one or more consensus sequence(s) comprising or consisting of the amino acid sequence K/R-(Z)₀₋₉-D/E-X-N-Y-S/T-(Z)₀₋₉-K/R (SEQ ID NO: 46) as defined above; for example one or more consensus sequence(s) selected from: K/R-D/E-X₁-N-X₂-S/T-Z₁-Z₂-K/R (SEQ ID NO 19), wherein X₁ and X₂ are independently any amino acid apart from proline, and wherein Z₁ and Z₂. are not lysine or arginine. In an embodiment, said consensus sequence comprises or consists of the amino acid sequence of SEQ ID NO: 20. In an embodiment, said consensus sequence comprises or consists of the amino acid sequence of any one of SEQ ID Nos: 42-45. In a preferred embodiment, said consensus sequence comprises or consists of the amino acid sequence of any one of SEQ ID Nos: 42-44.

In an embodiment, said modified Hla protein comprises more than one said consensus sequence, for example 2, 3, 4 or 5 consensus sequences. In a preferred embodiment, wherein multiple consensus sequences are present, the consensus sequences have different sequences in order that glycosylation at each individual site may be distinguished. In an embodiment, said modified Hla protein comprises at least one of the multiple consensus sequences at the N-terminal end and/or at the C-terminal end of the Hla sequence.

Because Hla is a toxin, it needs to be detoxified (i.e. rendered non-toxic to a mammal, e.g. human, when provided at a dosage suitable for protection) before it can be administered in vivo. A modified Hla protein of the invention may be genetically detoxified (i.e. by mutation). The genetically detoxified sequences may remove undesirable activities such as the ability to form a lipid-bilayer penetrating pore, membrane permeation, cell lysis, and cytolytic activity against human erythrocytes and other cells, in order to reduce toxicity, whilst retaining the ability to induce anti-Hla protective and/or neutralizing antibodies following administration to a human. For example, as described herein, a Hla protein may be altered so that it is biologically inactive whilst still maintaining its immunogenic epitopes. The modified Hla proteins of the invention may be genetically detoxified by one or more point mutations. For example, residues involved in pore formation been implicated in the lytic activity of Hla. In one aspect, the modified Hla proteins of the invention may be detoxified by amino acid substitutions as described in Menzies and Kernodle (Menzies and Kernodle, 1994, Infect Immun 62, 1843-1847), for example substitution of H35, H48, H114 and/or H259 with another amino acid such as lysine. For example, the modified Hla proteins of the invention may comprise at least one amino acid substitution selected from H35L, H114L or H259L, with reference to the amino acid sequence of SEQ ID NO: 13 (or an equivalent position in an amino acid sequence at least 80%, 85%, 90%, 92%, 95%, 96%, 97%, 98% or 99% identical to SEQ ID NO: 13). Preferably, the modified Hla protein comprises the substitution H35L (e.g. SEQ ID NO: 14).

Said modified Hla protein may thus be further modified in that the amino acid sequence comprises a detoxifying mutation, for example an amino acid substitution at position H35 (e.g. H35L) of SEQ ID NO: 13 or at an equivalent position within an amino acid sequence at least 80%, 85%, 90%, 92%, 95%, 96%, 97%, 98% or 99% identical to SEQ ID NO: 13 (e.g. SEQ ID NO: 14 and 15). An alternative detoxifying mutation is replacement of the stem region of the Hla monomer with PSGS, as for example in SEQ ID NO: 16. Exemplary modified sequences are those of SEQ ID NO: 31-34 and 36-39, in particular 31-33 and 36-38.

In an embodiment, said Hla sequence may be alternatively or additionally modified in that the amino acid sequence comprises amino acid substitutions at positions H48 and G122 of SEQ ID NO: 13 or at equivalent positions within an amino acid sequence at least 80%, 85%, 90%, 92%, 95%, 96%, 97%, 98% or 99% identical to SEQ ID NO: 13, wherein said substitutions are respectively H to C and G to C (e.g. SEQ ID NO: 15).

Accordingly, there is provided a modified Hla protein comprising an amino acid sequence of SEQ ID NO: 15 or an amino acid sequence at least 80%, 85%, 90%, 92%, 95%, 96%, 97%, 98% or 99% identical to SEQ ID NO: 15, wherein said modified Hla protein contains the following mutations: H35L, H48C and G122C modified in that the amino acid sequence comprises one or more consensus sequence(s) selected from SEQ ID Nos 19, 20 and 42-45, in particular 42-44, wherein said modified Hla protein contains the following mutations: H35L, H48C and G122C. Exemplary sequence are those of SEQ ID NO: 31-34 and 36-39, in particular 31-33 and 36-38.

These sequences may be modified by addition of a signal sequence and optionally insertion of an N-terminal serine and/or alanine for cloning purposes, as described herein. The sequences may further be modified to contain detoxifying mutations, such as any one or all of the detoxifying mutations described herein. A preferred detoxifying mutation is H35L of SEQ ID NO: 14 or 15.

In an embodiment, the modified Hla protein of the invention may be derived from an amino acid sequence at least 80%, 85%, 90%, 92%, 95%, 96%, 97%, 98% or 99% identical to SEQ ID NO: 13 which is an immunogenic fragment and/or a variant of SEQ ID NO: 13. In an embodiment, the modified Hla protein of the invention may be derived from an immunogenic fragment of SEQ ID NO: 13, 14 or 15 comprising at least about 15, at least about 20, at least about 40, or at least about 60 contiguous amino acid residues of the full length sequence, wherein said polypeptide is capable of eliciting an immune response specific for said amino acid sequence.

In an embodiment, the modified Hla protein of the invention may be derived from an amino acid sequence at least 80%, 85%, 90%, 92%, 95%, 96%, 97%, 98% or 99% identical to SEQ ID NO: 13 which is a variant of SEQ ID NO: 13 which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, the modified Hla protein of the present invention may be derived from a variant in which 1 to 10, 5 to 10, 1 to 5, 1 to 3, 1 to 2 or 1 amino acids are substituted, deleted, or added in any combination. For example, the modified Hla protein of the invention may be derived from an amino acid sequence which is a variant of any one of SEQ ID NOs. 13-16 in that it has one or two additional amino acids at the N terminus, for example an initial N-terminal SA (e.g. SEQ ID NO: 36-39). The modified Hla protein may additionally or alternatively have one or more additional amino acids at the C terminus, for example 1, 2, 3, 4, 5, or 6 amino acids. Such additional amino acids may include a peptide tag to assist in purification and include for example GSHRHR (e.g. SEQ ID NOs 36-39).

In an embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated based on the method described by Jameson and Wolf (CABIOS 4:181-186 [1988]).

The term “modified Hla protein” refers to a Hla acid sequence (for example, having a Hla amino acid sequence of SEQ ID NO: 13 or an amino acid sequence at least 80%, 85%, 90%, 91%, 92%, 93%, 94%, 95%, 96%, 97%, 98% or 99% A identical to SEQ ID NO: 13), which Hla amino acid sequence may be a wild-type mature Hla amino acid sequence (for example, a wild-type amino acid sequence of SEQ ID NO: 13), which has been modified by the addition, substitution or deletion of one or more amino acids (for example, substitution of H48 and G122 of SEQ ID NO: 13 with cysteine, substitution of H35 of SEQ ID NO: 1 with lysine, addition (insertion) of a consensus sequence(s) with amino acid sequence SEQ ID NO:19 or 46; or by substitution of one or more amino acids by a consensus sequence(s) a consensus sequence(s) with amino acid sequence SEQ ID NO:19 or 46. The modified Hla protein may also comprise further modifications (additions, substitutions, deletions) as well as the addition or substitution of one or more consensus sequence(s). For example, a signal sequence and/or peptide tag may be added. Additional amino acids at the N and/or C-terminal may be included to aid in cloning (for example, after the signal sequence or before the peptide tag, where present). In an embodiment, the modified Hla protein of the invention may be a non-naturally occurring Hla protein.

In an embodiment of the invention, one or more amino acids (e.g. 1-7 amino acids, e.g. one amino acid) of the modified Hla amino acid sequence (for example, having an amino acid sequence of SEQ ID NO: 13 or a Hla amino acid sequence at least 80%, 85%, 90%, 91%, 92%, 93%, 94%, 95%, 96%, 97%, 98% or 99% identical to SEQ ID NO: 13, e.g. SEQ ID Nos 14-16) have been substituted by a consensus sequence(s) with amino acid sequence SEQ ID NO:19 or 46, for example SEQ ID NO 20 or 42-44 or 47, in particular SEQ ID Nos: 42-44. For example, a single amino acid in the Hla amino acid sequence (e.g. SEQ ID NO: 13) may be replaced with a said consensus sequence (e.g. SEQ ID NOs: 30-39). In an embodiment, said substituted amino acid is at the position corresponding to position K131 of SEQ ID NO: 13. Alternatively, 2, 3, 4, 5, 6 or 7 amino acids in the Hla amino acid sequence (e.g. SEQ ID NO: 13 or a Hla amino acid sequence at least 80%, 85%, 90%, 92%, 95%, 96%, 97%, 98% or 99% A identical to SEQ ID NO: 13) may be replaced with said consensus sequence (e.g. SEQ ID NOs: 51-53) In an embodiment, said substituted amino acids are 2 or more amino acids selected from among amino acids at the N-terminal end, at the C-terminal end, and at the position 131 of SEQ ID NO: 13.

Introduction of a consensus sequence(s) enables the modified Hla protein to be glycosylated. Thus, the present invention also provides a modified Hla protein of the invention wherein the modified Hla protein is glycosylated. In specific embodiments, the consensus sequences are introduced into specific regions of the Hla amino acid sequence, e.g. surface structures of the protein, at the N or C termini of the protein, and/or in loops that are stabilized by disulfide bridges. In an aspect of the invention, the position of the consensus sequence(s) provides improved glycosylation, for example increased yield. In an embodiment, a consensus sequence has been added or substituted for one or more amino acid residues or in place of amino acid residue K131 of SEQ ID NO: 13 or in an equivalent position in an amino acid sequence at least 80%, 85%, 90%, 92%, 95%, 96%, 97%, 98% or 99% identical to SEQ ID NO: 13 (e.g. in an equivalent position in the amino acid sequence of SEQ ID Nos: 14-16), e.g. SEQ ID Nos: 30-39.

A person skilled in the art will understand that when the Hla amino acid sequence is a variant and/or fragment of an amino acid sequence of SEQ ID NO: 2, such as an amino acid sequence at least 80%, 85%, 90%, 92%, 95%, 96%, 97%, 98% or 99% A identical to SEQ ID NO: 2, the reference to “between amino acids . . . ” refers to the position that would be equivalent to the defined position, if this sequence was lined up with an amino acid sequence of SEQ ID NO: 1 in order to maximise the sequence identity between the two sequences Sequence alignment tools are described above. The addition or deletion of amino acids from the variant and/or fragment of SEQ ID NO: 13 could lead to a difference in the actual amino acid position of the consensus sequence in the mutated sequence, however, by lining the mutated sequence up with the reference sequence, the amino acid in in an equivalent position to the corresponding amino acid in the reference sequence can be identified and hence the appropriate position for addition or substitution of the consensus sequence can be established.

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein. Thus, in an embodiment, the present invention provides a modified Hla protein having an amino acid sequence wherein the amino acids corresponding to H48 and G122 of SEQ ID NO 13 or equivalent positions in an Hla amino acid sequence at least 80%, 85%, 90%, 92%, 95%, 96%, 97%, 98% or 99% A identical to SEQ ID NO: 13 have been substituted by cysteine, and wherein a glycosylation site has been recombinantly introduced into the Hla amino acid sequence of SEQ ID NO: 13 or a Hla amino acid sequence at least 80%, 85%, 90%, 92%, 95%, 96%, 97%, 98% or 99% identical to SEQ ID NO: 13.

In an embodiment, the modified Hla protein of the invention further comprises a “peptide tag” or “tag”, i.e. a sequence of amino acids that allows for the isolation and/or identification of the modified Hla protein. For example, adding a tag to a modified Hla protein of the invention can be useful in the purification of that protein and, hence, the purification of conjugate vaccines comprising the tagged modified Hla protein. Exemplary tags that can be used herein include, without limitation, histidine (HIS) tags. I one embodiment, the tag is a hexa-histidine tag. In another embodiment, the tag is a HR tag, for example an HRHR tag. In certain embodiments, the tags used herein are removable, e.g. removal by chemical agents or by enzymatic means, once they are no longer needed, e.g. after the protein has been purified. Optionally the peptide tag is located at the C-terminus of the amino acid sequence. Optionally the peptide tag comprises six histidine residues at the C-terminus of the amino acid sequence. Optionally the peptide tag comprises four HR residues (HRHR) at the C-terminus of the amino acid sequence. The peptide tag may be comprised or be preceded by one, two or more additional amino acid residues, for example alanine, serine and/or glycine residues, e.g. GS. Exemplary such sequences are SEQ ID Nos: 36-39.

In an embodiment, the modified Hla protein of the invention comprises a signal sequence which is capable of directing the carrier protein to the periplasm of a host cell (e.g. bacterium). In a specific embodiment, the signal sequence is from S. flexneri flagellin (FlgI) [MIKFLSALILLLVTTAAQA (SEQ ID NO: 21)]. In other embodiments, the signal sequence is from E. coli outer membrane porin A (OmpA) [MKKTAIAIAVALAGFATVAQA (SEQ ID NO: 22)], E. coli maltose binding protein (MalE) [MKIKTGARILALSALTTMMFSASALA (SEQ ID NO: 23)], Pectobacterium carotovorum pectate lyase (PelB) [MKYLLPTAAAGLLLLAAQPAMA (SEQ ID NO: 24], heat labile E. coli enterotoxin LTIIb [MSFKKIIKAFVIMAALVSVQAHA (SEQ ID NO: 25)], Bacillus subtilis endoxylanase XynA [MFKFKKKFLVGLTAAFMSISMFSATASA (SEQ ID NO: 26)], E. coli DsbA [MKKIWLALAGLVLAFSASA (SEQ ID NO: 27)], TolB [MKQALRVAFGFLILWASVLHA (SEQ ID NO: 28)] or S. agalactiae SipA [MKMNKKVLLTSTMAASLLSVASVQAS (SEQ ID NO: 29)]. In an embodiment, the signal sequence has an amino acid sequence at least 80%, 85%, 90%, 92%, 95%, 96%, 97%, 98%, 99% or 100% identical to a SEQ ID NO: 21-29. In one aspect, the signal sequence has an amino acid sequence at least 80%, 85%, 90%, 95%, 96%, 97%, 98%, 99% or 100% identical to E. coli flagellin signal sequence (FlgI) [MIKFLSALILLLVTTAAQA (SEQ ID NO: 21)]. Exemplary modified Hla sequences comprising a signal sequence are SEQ ID NOs: 35-39.

In an embodiment, a serine and/or alanine residue is added between the signal sequence and the start of the sequence of the mature protein, e.g. SA or S, preferably S. Such a reside or residues have the advantage of leading to more efficient cleavage of the leader sequence.

ClfA: Clumping Factor A from Staphylococcus aureus

In an embodiment, the carrier protein is clumping factor A (ClfA) from a staphylococcal bacterium, in particular S. aureus. ClfA has been used as carrier protein for S aureus capsular polysaccharide (CP8) and the ClfA-CP8 conjugate was able to induce functional antibodies to both ClfA and CP8 and had protective effect in animal models. ClfA contains a 520 amino acid N-terminal A domain (the Fibrinogen Binding Region), which comprises three separately folded subdomains N1, N2 and N3. The A domain is followed by a serine-aspartate dipeptide repeat region and a cell wall- and membrane-spanning region, which contains the LPDTG-motif for sortase-promoted anchoring to the cell wall. When used as an antigen or carrier protein, only the N1-N3 (SEQ ID NO: 10) or N2/N3 (SEQ ID No: 11) domains are used.

Said ClfA may thus comprise the amino acid sequence of SEQ ID NO: 10 or 11 or an amino acid sequence at least 80%, 85%, 90%, 92%, 95%, 96%, 97%, 98% or 99% identical to SEQ ID NO: 10 or 11.

The ClfA protein may be further modified to reduce its fibrinogen binding activity. Thus the ClfA protein may further comprise at least one amino acid substitution selected from P116 to S and Y118 to A with reference to the amino acid sequence of SEQ ID NO: 11 (corresponding to positions P336 and Y338 in the sequence of SEQ ID NO: 10) or an equivalent position in an amino acid sequence at least 80%, 85%, 90%, 92%, 95%, 96%, 97%, 98% or 99% identical to SEQ ID NO: 11.

Accordingly, there is provided in one aspect of the present invention, a modified ClfA protein comprising an amino acid sequence of SEQ ID NOs: 10, 11 or 12 or an amino acid sequence at least 80%, 85%, 90%, 92%, 95%, 96%, 97%, 98% or 99% identical to SEQ ID NOs: 10, 11 or 12, modified in that it comprises one or more consensus sequence(s) comprising or consisting of the amino acid sequence K/R-(Z)₀₋₉-D/E-X-N-Y-S/T-(Z)₀₋₉-K/R (SEQ ID NO: 46) as defined above; for example one or more consensus sequence(s) having the amino acid sequence K/R-D/E-X₁-N-X₂-S/T-Z₁-Z₂-K/R (SEQ ID NO 19), wherein X₁ and X₂ are independently any amino acid apart from proline, and wherein Z₁ and Z₂. are not lysine or arginine. In an embodiment, said consensus sequence comprises or consists of the amino acid sequence of SEQ ID NO: 20. In an embodiment, said consensus sequence comprises or consists of the amino acid sequence of any one of SEQ ID Nos: 42-45. In a preferred embodiment, said consensus sequence comprises or consists of the amino acid sequence of any one of SEQ ID Nos: 42-44.

In an embodiment, said modified ClfA protein comprises more than one said consensus sequence, for example 2, 3, 4 or 5 consensus sequences. In a preferred embodiment, wherein multiple consensus sequences are present, the consensus sequences have different sequences in order that glycosylation at each individual site may be distinguished.

In an embodiment, the modified ClfA protein of the invention may be derived from an amino acid sequence at least 80%, 85%, 90%, 92%, 95%, 96%, 97%, 98% or 99% identical to SEQ ID NO:s: 10, 11 or 12 which is an immunogenic fragment and/or a variant of SEQ ID Nos: 10, 11 or 12. In an embodiment, the modified ClfA protein of the invention may be derived from an immunogenic fragment of SEQ ID Nos: 10, 11 or 12 comprising at least about 15, at least about 20, at least about 40, or at least about 60 contiguous amino acid residues of the full length sequence, wherein said polypeptide is capable of eliciting an immune response specific for said amino acid sequence.

In an embodiment, the modified ClfA protein of the invention may be derived from an amino acid sequence at least 80%, 85%, 90%, 92%, 95%, 96%, 97%, 98% or 99% identical to SEQ ID Nos: 10, 11 or 12 which is a variant of SEQ ID Nos: 10, 11 or 12 which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, the modified ClfA protein of the present invention may be derived from a variant in which 1 to 10, 5 to 10, 1 to 5, 1 to 3, 1 to 2 or 1 amino acid are substituted, deleted, or added in any combination.

In an embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated based on the method described by Jameson and Wolf (CABIOS 4:181-186 [1988]).

The term “modified ClfA protein” refers to a ClfA amino acid sequence (for example, having a ClfA amino acid sequence of SEQ ID NO: 10, 11 or 12 or an amino acid sequence at least 80%, 85%, 90%, 91%, 92%, 93%, 94%, 95%, 96%, 97%, 98% or 99% identical to SEQ ID NO: 10, 11 or 12), which ClfA amino acid sequence has been modified by the addition, substitution or deletion of one or more amino acids (for example, addition (insertion) of a consensus sequence(s) with amino acid sequence SEQ ID NO:19 or 46; or by substitution of one or more amino acids by a consensus sequence(s) with amino acid sequence SEQ ID NO:19 or 46). The modified ClfA protein may also comprise further modifications (additions, substitutions, deletions) as well as the addition or substitution of one or more consensus sequence(s). For example, a signal sequence and/or peptide tag may be added. Additional amino acids at the N and/or C-terminal may be included to aid in cloning (for example, after the signal sequence or before the peptide tag, where present). In an embodiment, the modified ClfA protein of the invention may be a non-naturally occurring ClfA protein.

In an embodiment of the invention, one or more amino acids (e.g. 1-7 amino acids, e.g. one amino acid) of the modified ClfA amino acid sequence (for example, having an amino acid sequence of SEQ ID NO: 10, 11 or 12 or a ClfA amino acid sequence at least 80%, 85%, 90%, 91%, 92%, 93%, 94%, 95%, 96%, 97%, 98% or 99% identical to 10, 11 or 12) have been substituted by a consensus sequence(s) with amino acid sequence SEQ ID NO:19 or 46, for example SEQ ID NO 20 or 42-45 or 47, in particular SEQ ID Nos: 42-44. For example, a single amino acid in the ClfA amino acid sequence (e.g. SEQ ID NO: 10, 11 or 12) may be replaced with a said consensus sequence. Alternatively, 2, 3, 4, 5, 6 or 7 amino acids in the EPA amino acid sequence (e.g. SEQ ID NO: 10, 11 or 12 or a ClfA amino acid sequence at least 80%, 85%, 90%, 92%, 95%, 96%, 97%, 98% or 99% identical to SEQ ID NO: 10, 11 or 12) may be replaced with said consensus sequence.

Introduction of a consensus sequence(s) enables the modified ClfA protein to be glycosylated. Thus, the present invention also provides a modified ClfA protein of the invention wherein the modified ClfA protein is glycosylated. In specific embodiments, the consensus sequences are introduced into specific regions of the ClfA amino acid sequence, e.g. surface structures of the protein, at the N or C termini of the protein, and/or in loops that are stabilized by disulfide bridges.

A person skilled in the art will understand that when the ClfA amino acid sequence is a variant and/or fragment of an amino acid sequence of SEQ ID NO: 10, 11 or 12, such as an amino acid sequence at least 80%, 85%, 90%, 92%, 95%, 96%, 97%, 98% or 99% identical to SEQ ID NO: 10, 11 or 12, the reference to “between amino acids . . . ” refers to the position that would be equivalent to the defined position, if this sequence was lined up with an amino acid sequence of SEQ ID NO: 10, 11 or 12 in order to maximise the sequence identity between the two sequences Sequence alignment tools are described above. The addition or deletion of amino acids from the variant and/or fragment of SEQ ID NO: 10, 11 or 12 could lead to a difference in the actual amino acid position of the consensus sequence in the mutated sequence, however, by lining the mutated sequence up with the reference sequence, the amino acid in in an equivalent position to the corresponding amino acid in the reference sequence can be identified and hence the appropriate position for addition or substitution of the consensus sequence can be established.

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein.

In an embodiment, the modified ClfA protein of the invention further comprises a “peptide tag” or “tag”, i.e. a sequence of amino acids that allows for the isolation and/or identification of the modified ClfA protein. For example, adding a tag to a modified EPA protein of the invention can be useful in the purification of that protein and, hence, the purification of conjugate vaccines comprising the tagged modified ClfA protein. Exemplary tags that can be used herein include, without limitation, histidine (HIS) tags. I one embodiment, the tag is a hexa-histidine tag. In certain embodiments, the tags used herein are removable, e.g. removal by chemical agents or by enzymatic means, once they are no longer needed, e.g. after the protein has been purified. Optionally the peptide tag is located at the C-terminus of the amino acid sequence. Optionally the peptide tag comprises six histidine residues at the C-terminus of the amino acid sequence. The peptide tag may comprise or be preceded by one, two or more additional amino acid residues, for example alanine, serine and/or glycine residues, e.g. GS.

### CRM197: Non-Toxic Mutant of Diphtheria Toxin.

In an embodiment, the carrier protein is CRM197, a genetically detoxified mutant of diphtheria toxin having a single point mutation G52E compared to diphtheria toxin. CRM197 is a widely used and well tested carrier protein which has been used in several commercialised vaccines. The amino acid sequence of DT is shown in SEQ ID NO: 4 and that of CRM197 is shown in SEQ ID NO: 5.

Accordingly, there is provided in one aspect of the present invention, a modified CRM197 protein comprising an amino acid sequence of SEQ ID NO: 5 or an amino acid sequence at least 80%, 85%, 90%, 92%, 95%, 96%, 97%, 98% or 99% A identical to SEQ ID NO: 5, modified in that it comprises one or more consensus sequence(s) comprising or consisting of the amino acid sequence K/R-(Z)₀₋₉-D/E-X-N-Y-S/T-(Z)₀₋₉-K/R (SEQ ID NO: 46) as defined above; for example one or more consensus sequence(s) having the amino acid sequence K/R-D/E-X₁-N-X₂-S/T-Z₁-Z₂-K/R (SEQ ID NO 19), wherein X₁ and X₂ are independently any amino acid apart from proline, and wherein Z₁ and Z₂. are not lysine or arginine. In an embodiment, said consensus sequence comprises or consists of the amino acid sequence of SEQ ID NO: 20. In an embodiment, said consensus sequence comprises or consists of the amino acid sequence of any one of SEQ ID Nos: 42-45. In a preferred embodiment, said consensus sequence comprises or consists of the amino acid sequence of any one of SEQ ID Nos: 42-44.

In an embodiment, said modified CRM197 protein comprises more than one said consensus sequence, for example 2, 3, 4 or 5 consensus sequences. In a preferred embodiment, wherein multiple consensus sequences are present, the consensus sequences have different sequences in order that glycosylation at each individual site may be distinguished.

In an embodiment, the modified CRM197 protein of the invention may be derived from an amino acid sequence at least 80%, 85%, 90%, 92%, 95%, 96%, 97%, 98% or 99% identical to SEQ ID NO: 5 which is an immunogenic fragment and/or a variant of SEQ ID NO: 5. In an embodiment, the modified CRM197 protein of the invention may be derived from an immunogenic fragment of SEQ ID NO: 5 comprising at least about 15, at least about 20, at least about 40, or at least about 60 contiguous amino acid residues of the full length sequence, wherein said polypeptide is capable of eliciting an immune response specific for said amino acid sequence.

In an embodiment, the modified CRM197 protein of the invention may be derived from an amino acid sequence at least 80%, 85%, 90%, 92%, 95%, 96%, 97%, 98% or 99% identical to SEQ ID NO: 5 which is a variant of SEQ ID NO: 5 which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, the modified CRM197 protein of the present invention may be derived from a variant in which 1 to 10, 5 to 10, 1 to 5, 1 to 3, 1 to 2 or 1 amino acid are substituted, deleted, or added in any combination.

In an embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated on the basis of the method described by Jameson and Wolf (CABIOS 4:181-186 [1988]).

The term “modified CRM197 protein” refers to a CRM197 acid sequence (for example, having a CRM197 amino acid sequence of SEQ ID NO: 5 or an amino acid sequence at least 80%, 85%, 90%, 91%, 92%, 93%, 94%, 95%, 96%, 97%, 98% or 99% identical to SEQ ID NO: 5 which has been modified by the addition, substitution or deletion of one or more amino acids (for example, addition (insertion) of a consensus sequence(s) with amino acid sequence SEQ ID NO:19 or 46; or by substitution of one or more amino acids by a consensus sequence(s) with amino acid sequence SEQ ID NO:19 or 46. The modified CRM197 protein may also comprise further modifications (additions, substitutions, deletions) as well as the addition or substitution of one or more consensus sequence(s). For example, a signal sequence and/or peptide tag may be added. Additional amino acids at the N and/or C-terminal may be included to aid in cloning (for example, after the signal sequence or before the peptide tag, where present).

In an embodiment of the invention, one or more amino acids (e.g. 1-7 amino acids, e.g. one amino acid) of the modified CRM197 amino acid sequence (for example, having an amino acid sequence of SEQ ID NO: 5) have been substituted by a consensus sequence(s) with amino acid sequence SEQ ID NO:19 or 46, for example SEQ ID NO 20 or 42-45 or 47, in particular SEQ ID Nos: 42-44. For example, a single amino acid in the CRM197 amino acid sequence (e.g. SEQ ID NO: 5) may be replaced with a said consensus sequence. Alternatively, 2, 3, 4, 5, 6 or 7 amino acids in the CRM197 amino acid sequence (e.g. SEQ ID NO: 5) may be replaced with said consensus sequence.

Introduction of a consensus sequence(s) enables the modified CRM197 protein to be glycosylated. Thus, the present invention also provides a modified CRM197 protein of the invention wherein the modified CRM197 protein is glycosylated. In specific embodiments, the consensus sequences are introduced into specific regions of the CRM197 amino acid sequence, e.g. surface structures of the protein, at the N or C termini of the protein, and/or in loops that are stabilized by disulfide bridges.

A person skilled in the art will understand that when the CRM197 amino acid sequence is a variant and/or fragment of an amino acid sequence of SEQ ID NO: 5, such as an amino acid sequence at least 80%, 85%, 90%, 92%, 95%, 96%, 97%, 98% or 99% A identical to SEQ ID NO: 5, the reference to “between amino acids . . . ” refers to the position that would be equivalent to the defined position, if this sequence was lined up with an amino acid sequence of SEQ ID NO: 5 in order to maximise the sequence identity between the two sequences Sequence alignment tools are described above. The addition or deletion of amino acids from the variant and/or fragment of SEQ ID NO: 5 could lead to a difference in the actual amino acid position of the consensus sequence in the mutated sequence, however, by lining the mutated sequence up with the reference sequence, the amino acid in in an equivalent position to the corresponding amino acid in the reference sequence can be identified and hence the appropriate position for addition or substitution of the consensus sequence can be established.

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein.

In an embodiment, the modified CRM197 protein of the invention further comprises a “peptide tag” or “tag”, i.e. a sequence of amino acids that allows for the isolation and/or identification of the modified CRM197 protein. For example, adding a tag to a modified CRM197 protein of the invention can be useful in the purification of that protein and, hence, the purification of conjugate vaccines comprising the tagged modified CRM197 protein. Exemplary tags that can be used herein include, without limitation, histidine (HIS) tags. In one embodiment, the tag is a hexa-histidine tag. In certain embodiments, the tags used herein are removable, e.g. removal by chemical agents or by enzymatic means, once they are no longer needed, e.g. after the protein has been purified. Optionally the peptide tag is located at the C-terminus of the amino acid sequence. Optionally the peptide tag comprises six histidine residues at the C-terminus of the amino acid sequence. The peptide tag may comprise or be preceded by one, two or more additional amino acid residues, for example alanine, serine and/or glycine residues, e.g. GS.

### Tetanus Toxin

Tetanus toxin (TT) produced by C. tetani cultures is widely used as a carrier after detoxification by formaldehyde inactivation. Fragments of TT which show lower toxicity have also been produced recombinant means.

Accordingly, there is provided in one aspect of the present invention, a modified TT protein comprising an amino acid sequence of SEQ ID NO: 3 or an amino acid sequence at least 80%, 85%, 90%, 92%, 95%, 96%, 97%, 98% or 99% A identical to SEQ ID NO: 3, modified in that it comprises one or more consensus sequence(s) comprising or consisting of the amino acid sequence K/R-(Z)₀₋₉-D/E-X-N-Y-S/T-(Z)₀₋₉-K/R (SEQ ID NO: 46) as defined above; for example one or more consensus sequence(s) having the amino acid sequence K/R-D/E-X₁-N-X₂-S/T-Z₁-Z₂-K/R (SEQ ID NO 19), wherein X₁ and X₂ are independently any amino acid apart from proline, and wherein Z₁ and Z₂. are not lysine or arginine. In an embodiment, said consensus sequence comprises or consists of the amino acid sequence of SEQ ID NO: 20. In an embodiment, said consensus sequence comprises or consists of the amino acid sequence of any one of SEQ ID Nos: 42-45. In a preferred embodiment, said consensus sequence comprises or consists of the amino acid sequence of any one of SEQ ID Nos: 42-44.

In an embodiment, said modified TT protein comprises more than one said consensus sequence, for example 2, 3, 4 or 5 consensus sequences. In a preferred embodiment, wherein multiple consensus sequences are present, the consensus sequences have different sequences in order that glycosylation at each individual site may be distinguished.

In an embodiment, the modified TT protein of the invention may be derived from an amino acid sequence at least 80%, 85%, 90%, 92%, 95%, 96%, 97%, 98% or 99% A identical to SEQ ID NO: 3 which is an immunogenic fragment and/or a variant of SEQ ID NO: 3. In an embodiment, the modified TT protein of the invention may be derived from an immunogenic fragment of SEQ ID NO: 3 or 2 comprising at least about 15, at least about 20, at least about 40, or at least about 60 contiguous amino acid residues of the full length sequence, wherein said polypeptide is capable of eliciting an immune response specific for said amino acid sequence.

In an embodiment, the modified TT protein of the invention may be derived from an amino acid sequence at least 80%, 85%, 90%, 92%, 95%, 96%, 97%, 98% or 99% identical to SEQ ID NO: 3 which is a variant of SEQ ID NO: 3 which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, the modified TT protein of the present invention may be derived from a variant in which 1 to 10, 5 to 10, 1 to 5, 1 to 3, 1 to 2 or 1 amino acid are substituted, deleted, or added in any combination.

In an embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated on the basis of the method described by Jameson and Wolf (CABIOS 4:181-186 [1988]).

The term “modified TT protein” refers to a TT acid sequence (for example, having a TT amino acid sequence of SEQ ID NO: 3 or an amino acid sequence at least 80%, 85%, 90%, 91%, 92%, 93%, 94%, 95%, 96%, 97%, 98% or 99% A identical to SEQ ID NO: 3), which TT amino acid sequence may be a wild-type mature TT amino acid sequence (for example, a wild-type amino acid sequence of SEQ ID NO: 3), which has been modified by the addition, substitution or deletion of one or more amino acids (for example, addition (insertion) of a consensus sequence(s) with amino acid sequence SEQ ID NO:19 or 46; or by substitution of one or more amino acids by a consensus sequence(s) with amino acid sequence SEQ ID NO:19 or 46. The modified TT protein may also comprise further modifications (additions, substitutions, deletions) as well as the addition or substitution of one or more consensus sequence(s). For example, a signal sequence and/or peptide tag may be added. Additional amino acids at the N and/or C-terminal may be included to aid in cloning (for example, after the signal sequence or before the peptide tag, where present). In an embodiment, the modified TT protein of the invention may be a non-naturally occurring TT protein.

In an embodiment of the invention, one or more amino acids (e.g. 1-7 amino acids, e.g. one amino acid) of the modified TT amino acid sequence (for example, having an amino acid sequence of SEQ ID NO: 3 or a TT amino acid sequence at least 80%, 85%, 90%, 91%, 92%, 93%, 94%, 95%, 96%, 97%, 98% or 99% identical to SEQ ID NO: 3) have been substituted by a consensus sequence(s) with amino acid sequence SEQ ID NO:19 or 46, for example SEQ ID NO 20 or 42-45 or 47, in particular SEQ ID Nos: 42-44. For example, a single amino acid in the TT amino acid sequence (e.g. SEQ ID NO: 3) may be replaced with a said consensus sequence. Alternatively, 2, 3, 4, 5, 6 or 7 amino acids in the TT amino acid sequence (e.g. SEQ ID NO: 3 or a TT amino acid sequence at least 80%, 85%, 90%, 92%, 95%, 96%, 97%, 98% or 99% identical to SEQ ID NO: 3) may be replaced with said consensus sequence.

Introduction of a consensus sequence(s) enables the modified TT protein to be glycosylated. Thus, the present invention also provides a modified TT protein of the invention wherein the modified TT protein is glycosylated. In specific embodiments, the consensus sequences are introduced into specific regions of the TT amino acid sequence, e.g. surface structures of the protein, at the N or C termini of the protein, and/or in loops that are stabilized by disulfide bridges.

A person skilled in the art will understand that when the TT amino acid sequence is a variant and/or fragment of an amino acid sequence of SEQ ID NO: 3, such as an amino acid sequence at least 80%, 85%, 90%, 92%, 95%, 96%, 97%, 98% or 99% A identical to SEQ ID NO: 3, the reference to “between amino acids . . . ” refers to the position that would be equivalent to the defined position, if this sequence was lined up with an amino acid sequence of SEQ ID NO: 3 in order to maximise the sequence identity between the two sequences Sequence alignment tools are described above. The addition or deletion of amino acids from the variant and/or fragment of SEQ ID NO: 3 could lead to a difference in the actual amino acid position of the consensus sequence in the mutated sequence, however, by lining the mutated sequence up with the reference sequence, the amino acid in in an equivalent position to the corresponding amino acid in the reference sequence can be identified and hence the appropriate position for addition or substitution of the consensus sequence can be established.

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein.

In an embodiment, the modified TT protein of the invention further comprises a “peptide tag” or “tag”, i.e. a sequence of amino acids that allows for the isolation and/or identification of the modified TT protein. For example, adding a tag to a modified TT protein of the invention can be useful in the purification of that protein and, hence, the purification of conjugate vaccines comprising the tagged modified TT protein. Exemplary tags that can be used herein include, without limitation, histidine (HIS) tags. In one embodiment, the tag is a hexa-histidine tag. In certain embodiments, the tags used herein are removable, e.g. removal by chemical agents or by enzymatic means, once they are no longer needed, e.g. after the protein has been purified. Optionally the peptide tag is located at the C-terminus of the amino acid sequence. Optionally the peptide tag comprises six histidine residues at the C-terminus of the amino acid sequence. The peptide tag may comprise or be preceded by one, two or more additional amino acid residues, for example alanine, serine and/or glycine residues, e.g. GS.

Hcp1: Protein Hcp1 from Pseudomonas aeruginosa

In an embodiment, the carrier protein is Hcp1 from Pseudomonas aeruginosa (Hcp1). Said Hcp1 may comprise the amino acid sequence of SEQ ID NO: 6 or an amino acid sequence at least 80%, 85%, 90%, 92%, 95%, 96%, 97%, 98% or 99% A identical to SEQ ID NO: 6.

Accordingly, there is provided in one aspect of the present invention, a modified Hcp1 protein comprising an amino acid sequence of SEQ ID NO: 6 or an amino acid sequence at least 80%, 85%, 90%, 92%, 95%, 96%, 97%, 98% or 99% A identical to SEQ ID NO: 6, modified in that it comprises one or more consensus sequence(s) comprising or consisting of the amino acid sequence K/R-(Z)₀₋₉-D/E-X-N-Y-S/T-(Z)₀₋₉-K/R (SEQ ID NO: 46) as defined above; for example one or more consensus sequence(s) having the amino acid sequence K/R-D/E-X₁-N-X₂-S/T-Z₁-Z₂-K/R (SEQ ID NO 19), wherein X₁ and X₂ are independently any amino acid apart from proline, and wherein Z₁ and Z₂. are not lysine or arginine. In an embodiment, said consensus sequence comprises or consists of the amino acid sequence of SEQ ID NO: 20. In an embodiment, said consensus sequence comprises or consists of the amino acid sequence of any one of SEQ ID Nos: 42-45. In a preferred embodiment, said consensus sequence comprises or consists of the amino acid sequence of any one of SEQ ID Nos: 42-44.

In an embodiment, said modified Hcp1 protein comprises more than one said consensus sequence, for example 2, 3, 4 or 5 consensus sequences. In a preferred embodiment, wherein multiple consensus sequences are present, the consensus sequences have different sequences in order that glycosylation at each individual site may be distinguished.

In an embodiment, the modified Hcp1 protein of the invention may be derived from an amino acid sequence at least 80%, 85%, 90%, 92%, 95%, 96%, 97%, 98% or 99% A identical to SEQ ID NO: 6 which is an immunogenic fragment and/or a variant of SEQ ID NO: 6. In an embodiment, the modified Hcp1 protein of the invention may be derived from an immunogenic fragment of SEQ ID NO: 6 comprising at least about 15, at least about 20, at least about 40, or at least about 60 contiguous amino acid residues of the full length sequence, wherein said polypeptide is capable of eliciting an immune response specific for said amino acid sequence.

In an embodiment, the modified Hcp1 protein of the invention may be derived from an amino acid sequence at least 80%, 85%, 90%, 92%, 95%, 96%, 97%, 98% or 99% A identical to SEQ ID NO: 6 which is a variant of SEQ ID NO: 6 which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, the modified Hcp1 protein of the present invention may be derived from a variant in which 1 to 10, 5 to 10, 1 to 5, 1 to 3, 1 to 2 or 1 amino acid are substituted, deleted, or added in any combination.

In an embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated on the basis of the method described by Jameson and Wolf (CABIOS 4:181-186 [1988]).

The term “modified Hcp1 protein” refers to a Hcp1 acid sequence (for example, having a Hcp1 amino acid sequence of SEQ ID NO: 6 or an amino acid sequence at least 80%, 85%, 90%, 91%, 92%, 93%, 94%, 95%, 96%, 97%, 98% or 99% identical to SEQ ID NO: 6), which Hcp1 amino acid has been modified by the addition, substitution or deletion of one or more amino acids (for example, addition (insertion) of a consensus sequence(s) with amino acid sequence SEQ ID NO:19 or 46; or by substitution of one or more amino acids by a consensus sequence(s) with amino acid sequence SEQ ID NO:19 or 46. The modified Hcp1 protein may also comprise further modifications (additions, substitutions, deletions) as well as the addition or substitution of one or more consensus sequence(s). For example, a signal sequence and/or peptide tag may be added. Additional amino acids at the N and/or C-terminal may be included to aid in cloning (for example, after the signal sequence or before the peptide tag, where present). In an embodiment, the modified Hcp1 protein of the invention may be a non-naturally occurring Hcp1 protein.

In an embodiment of the invention, one or more amino acids (e.g. 1-7 amino acids, e.g. one amino acid) of the modified Hcp1 amino acid sequence (for example, having an amino acid sequence of SEQ ID NO: 6 or a Hcp1 amino acid sequence at least 80%, 85%, 90%, 91%, 92%, 93%, 94%, 95%, 96%, 97%, 98% or 99% A identical to SEQ ID NO: 6) have been substituted by a consensus sequence(s) with amino acid sequence SEQ ID NO:19 or 46, for example SEQ ID NO 20 or 42-45 or 47, in particular SEQ ID Nos: 42-44. For example, a single amino acid in the Hcp1 amino acid sequence (e.g. SEQ ID NO: 6) may be replaced with a said consensus sequence. Alternatively, 2, 3, 4, 5, 6 or 7 amino acids in the HCP6 amino acid sequence (e.g. SEQ ID NO: 6 or a Hcp1 amino acid sequence at least 80%, 85%, 90%, 92%, 95%, 96%, 97%, 98% or 99% identical to SEQ ID NO: 6) may be replaced with said consensus sequence.

Introduction of a consensus sequence(s) enables the modified Hcp1 protein to be glycosylated. Thus, the present invention also provides a modified Hcp1 protein of the invention wherein the modified Hcp1 protein is glycosylated. In specific embodiments, the consensus sequences are introduced into specific regions of the Hcp1 amino acid sequence, e.g. surface structures of the protein, at the N or C termini of the protein, and/or in loops that are stabilized by disulfide bridges.

A person skilled in the art will understand that when the Hcp1 amino acid sequence is a variant and/or fragment of an amino acid sequence of SEQ ID NO: 6, such as an amino acid sequence at least 80%, 85%, 90%, 92%, 95%, 96%, 97%, 98% or 99% identical to SEQ ID NO: 6, the reference to “between amino acids . . . ” refers to the position that would be equivalent to the defined position, if this sequence was lined up with an amino acid sequence of SEQ ID NO: 6 in order to maximise the sequence identity between the two sequences Sequence alignment tools are described above. The addition or deletion of amino acids from the variant and/or fragment of SEQ ID NO: 6 could lead to a difference in the actual amino acid position of the consensus sequence in the mutated sequence, however, by lining the mutated sequence up with the reference sequence, the amino acid in in an equivalent position to the corresponding amino acid in the reference sequence can be identified and hence the appropriate position for addition or substitution of the consensus sequence can be established.

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein.

In an embodiment, the modified Hcp1 protein of the invention further comprises a “peptide tag” or “tag”, i.e. a sequence of amino acids that allows for the isolation and/or identification of the modified Hcp1 protein. For example, adding a tag to a modified Hcp1 protein of the invention can be useful in the purification of that protein and, hence, the purification of conjugate vaccines comprising the tagged modified Hcp1 protein. Exemplary tags that can be used herein include, without limitation, histidine (HIS) tags. In one embodiment, the tag is a hexa-histidine tag. In certain embodiments, the tags used herein are removable, e.g. removal by chemical agents or by enzymatic means, once they are no longer needed, e.g. after the protein has been purified. Optionally the peptide tag is located at the C-terminus of the amino acid sequence. Optionally the peptide tag comprises six histidine residues at the C-terminus of the amino acid sequence. The peptide tag may comprise or be preceded by one, two or more additional amino acid residues, for example alanine, serine and/or glycine residues, e.g. GS.

MBP: Maltose/Maltodextrin Binding Protein from Escherichia coli.

In an embodiment, the carrier protein is exotoxin A from Pseudomonas aeruginosa (MBP). Said MBP may comprise the amino acid sequence of SEQ ID NO: 8 or an amino acid sequence at least 80%, 85%, 90%, 92%, 95%, 96%, 97%, 98% or 99% identical to SEQ ID NO: 8.

Accordingly, there is provided in one aspect of the present invention, a modified MBP protein comprising an amino acid sequence of SEQ ID NO: 8 or an amino acid sequence at least 80%, 85%, 90%, 92%, 95%, 96%, 97%, 98% or 99% identical to SEQ ID NO: 8, modified in that it comprises one or more consensus sequence(s) comprising or consisting of the amino acid sequence K/R-(Z)₀₋₉-D/E-X-N-Y-S/T-(Z)₀₋₉-K/R (SEQ ID NO: 46) as defined above; for example one or more consensus sequence(s) having the amino acid sequence K/R-D/E-X₁-N-X₂-S/T-Z₁-Z₂-K/R (SEQ ID NO 19), wherein X₁ and X₂ are independently any amino acid apart from proline, and wherein Z₁ and Z₂. are not lysine or arginine. In an embodiment, said consensus sequence comprises or consists of the amino acid sequence of SEQ ID NO: 20. In an embodiment, said consensus sequence comprises or consists of the amino acid sequence of any one of SEQ ID Nos: 42-45. In a preferred embodiment, said consensus sequence comprises or consists of the amino acid sequence of any one of SEQ ID Nos: 42-44.

In an embodiment, said modified MBP protein comprises more than one said consensus sequence, for example 2, 3, 4 or 5 consensus sequences. In a preferred embodiment, wherein multiple consensus sequences are present, the consensus sequences have different sequences in order that glycosylation at each individual site may be distinguished.

In an embodiment, the modified MBP protein of the invention may be derived from an amino acid sequence at least 80%, 85%, 90%, 92%, 95%, 96%, 97%, 98% or 99% A identical to SEQ ID NO: 8 which is an immunogenic fragment and/or a variant of SEQ ID NO: 8. In an embodiment, the modified MBP protein of the invention may be derived from an immunogenic fragment of SEQ ID NO: 8 comprising at least about 15, at least about 20, at least about 40, or at least about 60 contiguous amino acid residues of the full length sequence, wherein said polypeptide is capable of eliciting an immune response specific for said amino acid sequence.

In an embodiment, the modified MBP protein of the invention may be derived from an amino acid sequence at least 80%, 85%, 90%, 92%, 95%, 96%, 97%, 98% or 99% A identical to SEQ ID NO: 8 which is a variant of SEQ ID NO: 8 which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, the modified MBP protein of the present invention may be derived from a variant in which 1 to 10, 5 to 10, 1 to 5, 1 to 3, 1 to 2 or 1 amino acid are substituted, deleted, or added in any combination.

In an embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated based on the method described by Jameson and Wolf (CABIOS 4:181-186 [1988]).

The term “modified MBP protein” refers to a MBP acid sequence (for example, having a MBP amino acid sequence of SEQ ID NO: 8 or an amino acid sequence at least 80%, 85%, 90%, 91%, 92%, 93%, 94%, 95%, 96%, 97%, 98% or 99% A identical to SEQ ID NO: 8), which MBP amino acid sequence may be a wild-type mature MBP amino acid sequence (for example, a wild-type amino acid sequence of SEQ ID NO: 8), which has been modified by the addition, substitution or deletion of one or more amino acids (for example, addition (insertion) of a consensus sequence(s) with amino acid sequence SEQ ID NO:19 or 46; or by substitution of one or more amino acids by a consensus sequence(s) with amino acid sequence SEQ ID NO:19 or 46. The modified MBP protein may also comprise further modifications (additions, substitutions, deletions) as well as the addition or substitution of one or more consensus sequence(s). For example, a signal sequence and/or peptide tag may be added. Additional amino acids at the N and/or C-terminal may be included to aid in cloning (for example, after the signal sequence or before the peptide tag, where present). In an embodiment, the modified MBP protein of the invention may be a non-naturally occurring MBP protein.

In an embodiment of the invention, one or more amino acids (e.g. 1-7 amino acids, e.g. one amino acid) of the modified MBP amino acid sequence (for example, having an amino acid sequence of SEQ ID NO: 8 or a MBP amino acid sequence at least 80%, 85%, 90%, 91%, 92%, 93%, 94%, 95%, 96%, 97%, 98% or 99% identical to SEQ ID NO: 8) have been substituted by a consensus sequence(s) with amino acid sequence SEQ ID NO:19 or 46, for example SEQ ID NO 20 or 42-45 or 47, in particular SEQ ID Nos: 42-44. For example, a single amino acid in the MBP amino acid sequence (e.g. SEQ ID NO: 8) may be replaced with a said consensus sequence. Alternatively, 2, 3, 4, 5, 6 or 7 amino acids in the MBP amino acid sequence (e.g. SEQ ID NO: 8 or a MBP amino acid sequence at least 80%, 85%, 90%, 92%, 95%, 96%, 97%, 98% or 99% A identical to SEQ ID NO: 8) may be replaced with said consensus sequence.

Introduction of a consensus sequence(s) enables the modified MBP protein to be glycosylated. Thus, the present invention also provides a modified MBP protein of the invention wherein the modified MBP protein is glycosylated. In specific embodiments, the consensus sequences are introduced into specific regions of the MBP amino acid sequence, e.g. surface structures of the protein, at the N or C termini of the protein, and/or in loops that are stabilized by disulfide bridges.

A person skilled in the art will understand that when the MBP amino acid sequence is a variant and/or fragment of an amino acid sequence of SEQ ID NO: 8, such as an amino acid sequence at least 80%, 85%, 90%, 92%, 95%, 96%, 97%, 98% or 99% A identical to SEQ ID NO: 8, the reference to “between amino acids . . . ” refers to the position that would be equivalent to the defined position, if this sequence was lined up with an amino acid sequence of SEQ ID NO: 8 in order to maximise the sequence identity between the two sequences Sequence alignment tools are described above. The addition or deletion of amino acids from the variant and/or fragment of SEQ ID NO: 8 could lead to a difference in the actual amino acid position of the consensus sequence in the mutated sequence, however, by lining the mutated sequence up with the reference sequence, the amino acid in in an equivalent position to the corresponding amino acid in the reference sequence can be identified and hence the appropriate position for addition or substitution of the consensus sequence can be established.

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein.

In an embodiment, the modified MBP protein of the invention further comprises a “peptide tag” or “tag”, i.e. a sequence of amino acids that allows for the isolation and/or identification of the modified MBP protein. For example, adding a tag to a modified MBP protein of the invention can be useful in the purification of that protein and, hence, the purification of conjugate vaccines comprising the tagged modified MBP protein. Exemplary tags that can be used herein include, without limitation, histidine (HIS) tags. I one embodiment, the tag is a hexa-histidine tag. In certain embodiments, the tags used herein are removable, e.g. removal by chemical agents or by enzymatic means, once they are no longer needed, e.g. after the protein has been purified. Optionally the peptide tag is located at the C-terminus of the amino acid sequence. Optionally the peptide tag comprises six histidine residues at the C-terminus of the amino acid sequence. The peptide tag may comprise or be preceded by one, two or more additional amino acid residues, for example alanine, serine and/or glycine residues, e.g. GS.

MtrE: Membrane Transporter E from Neisseria gonorrhoeae.

In an embodiment, the carrier protein is Membrane Transporter E from Neisseria gonorrhoeae (MtrE). Said MtrE may comprise the amino acid sequence of SEQ ID NO: 9 or an amino acid sequence at least 80%, 85%, 90%, 92%, 95%, 96%, 97%, 98% or 99% A identical to SEQ ID NO: 9.

Accordingly, there is provided in one aspect of the present invention, a modified MtrE protein comprising an amino acid sequence of SEQ ID NO: 9 or an amino acid sequence at least 80%, 85%, 90%, 92%, 95%, 96%, 97%, 98% or 99% A identical to SEQ ID NO: 9, modified in that it comprises one or more consensus sequence(s) comprising or consisting of the amino acid sequence K/R-(Z)₀₋₉-D/E-X-N-Y-S/T-(Z)₀₋₉-K/R (SEQ ID NO: 46) as defined above; for example one or more consensus sequence(s) having the amino acid sequence K/R-D/E-X₁-N-X₂-S/T-Z₁-Z₂-K/R (SEQ ID NO 19), wherein X₁ and X₂ are independently any amino acid apart from proline, and wherein Z₁ and Z₂. are not lysine or arginine. In an embodiment, said consensus sequence comprises or consists of the amino acid sequence of SEQ ID NO: 20. In an embodiment, said consensus sequence comprises or consists of the amino acid sequence of any one of SEQ ID Nos: 42-45. In a preferred embodiment, said consensus sequence comprises or consists of the amino acid sequence of any one of SEQ ID Nos: 42-44.

In an embodiment, said modified MtrE protein comprises more than one said consensus sequence, for example 2, 3, 4 or 5 consensus sequences. In a preferred embodiment, wherein multiple consensus sequences are present, the consensus sequences have different sequences in order that glycosylation at each individual site may be distinguished.

In an embodiment, the modified MtrE protein of the invention may be derived from an amino acid sequence at least 80%, 85%, 90%, 92%, 95%, 96%, 97%, 98% or 99% A identical to SEQ ID NO: 9 which is an immunogenic fragment and/or a variant of SEQ ID NO: 9. In an embodiment, the modified MtrE protein of the invention may be derived from an immunogenic fragment of SEQ ID NO: 9 comprising at least about 15, at least about 20, at least about 40, or at least about 60 contiguous amino acid residues of the full length sequence, wherein said polypeptide is capable of eliciting an immune response specific for said amino acid sequence.

In an embodiment, the modified MtrE protein of the invention may be derived from an amino acid sequence at least 80%, 85%, 90%, 92%, 95%, 96%, 97%, 98% or 99% identical to SEQ ID NO: 9 which is a variant of SEQ ID NO: 9 which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, the modified MtrE protein of the present invention may be derived from a variant in which 1 to 10, 5 to 10, 1 to 5, 1 to 3, 1 to 2 or 1 amino acid are substituted, deleted, or added in any combination.

In an embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated based on the method described by Jameson and Wolf (CABIOS 4:181-186 [1988]).

The term “modified MtrE protein” refers to a MtrE amino acid sequence (for example, having a MtrE amino acid sequence of SEQ ID NO: 9 or an amino acid sequence at least 80%, 85%, 90%, 91%, 92%, 93%, 94%, 95%, 96%, 97%, 98% or 99% identical to SEQ ID NO: 9), which MtrE amino acid sequence may be a wild-type mature MtrE amino acid sequence (for example, a wild-type amino acid sequence of SEQ ID NO: 9), which has been modified by the addition, substitution or deletion of one or more amino acids (for example, addition (insertion) of a consensus sequence(s) with amino acid sequence SEQ ID NO:19 or 46; or by substitution of one or more amino acids by a consensus sequence(s) with amino acid sequence SEQ ID NO:19 or 46. The modified MtrE protein may also comprise further modifications (additions, substitutions, deletions) as well as the addition or substitution of one or more consensus sequence(s). For example, a signal sequence and/or peptide tag may be added. Additional amino acids at the N and/or C-terminal may be included to aid in cloning (for example, after the signal sequence or before the peptide tag, where present). In an embodiment, the modified MtrE protein of the invention may be a non-naturally occurring MtrE protein.

In an embodiment of the invention, one or more amino acids (e.g. 1-7 amino acids, e.g. one amino acid) of the modified MtrE amino acid sequence (for example, having an amino acid sequence of SEQ ID NO: 9 or a MtrE amino acid sequence at least 80%, 85%, 90%, 91%, 92%, 93%, 94%, 95%, 96%, 97%, 98% or 99% A identical to SEQ ID NO: 9, e.g. SEQ ID No 9) have been substituted by a consensus sequence(s) with amino acid sequence SEQ ID NO:19 or 46, for example SEQ ID NO 20 or 42-45 or 47, in particular SEQ ID Nos: 42-44. For example, a single amino acid in the MtrE amino acid sequence (e.g. SEQ ID NO: 9) may be replaced with a said consensus sequence. Alternatively, 2, 3, 4, 5, 6 or 7 amino acids in the MtrE amino acid sequence (e.g. SEQ ID NO: 9 or a MtrE amino acid sequence at least 80%, 85%, 90%, 92%, 95%, 96%, 97%, 98% or 99% identical to SEQ ID NO: 9) may be replaced with said consensus sequence.

Introduction of a consensus sequence(s) enables the modified MtrE protein to be glycosylated. Thus, the present invention also provides a modified MtrE protein of the invention wherein the modified MtrE protein is glycosylated. In specific embodiments, the consensus sequences are introduced into specific regions of the MtrE amino acid sequence, e.g. surface structures of the protein, at the N or C termini of the protein, and/or in loops that are stabilized by disulfide bridges.

A person skilled in the art will understand that when the MtrE amino acid sequence is a variant and/or fragment of an amino acid sequence of SEQ ID NO: 9, such as an amino acid sequence at least 80%, 85%, 90%, 92%, 95%, 96%, 97%, 98% or 99% A identical to SEQ ID NO: 9, the reference to “between amino acids . . . ” refers to the position that would be equivalent to the defined position, if this sequence was lined up with an amino acid sequence of SEQ ID NO: 9 in order to maximise the sequence identity between the two sequences Sequence alignment tools are described above. The addition or deletion of amino acids from the variant and/or fragment of SEQ ID NO: 9 could lead to a difference in the actual amino acid position of the consensus sequence in the mutated sequence, however, by lining the mutated sequence up with the reference sequence, the amino acid in in an equivalent position to the corresponding amino acid in the reference sequence can be identified and hence the appropriate position for addition or substitution of the consensus sequence can be established.

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein.

In an embodiment, the modified MtrE protein of the invention further comprises a “peptide tag” or “tag”, i.e. a sequence of amino acids that allows for the isolation and/or identification of the modified MtrE protein. For example, adding a tag to a modified MtrE protein of the invention can be useful in the purification of that protein and, hence, the purification of conjugate vaccines comprising the tagged modified MtrE protein. Exemplary tags that can be used herein include, without limitation, histidine (HIS) tags. I one embodiment, the tag is a hexa-histidine tag. In certain embodiments, the tags used herein are removable, e.g. removal by chemical agents or by enzymatic means, once they are no longer needed, e.g. after the protein has been purified. Optionally the peptide tag is located at the C-terminus of the amino acid sequence. Optionally the peptide tag comprises six histidine residues at the C-terminus of the amino acid sequence. The peptide tag may comprise or be preceded by one, two or more additional amino acid residues, for example alanine, serine and/or glycine residues, e.g. GS.

PspA, Phage Shock Protein a from Escherichia coli.

In an embodiment, the carrier protein is phage shock protein A from Pseudomonas aeruginosa (PspA). Said PspA may comprise the amino acid sequence of SEQ ID NO: 7 or an amino acid sequence at least 80%, 85%, 90%, 92%, 95%, 96%, 97%, 98% or 99% A identical to SEQ ID NO: 7.

Accordingly, there is provided in one aspect of the present invention, a modified PspA protein comprising an amino acid sequence of SEQ ID NO: 7 or an amino acid sequence at least 80%, 85%, 90%, 92%, 95%, 96%, 97%, 98% or 99% A identical to SEQ ID NO: 7, modified in that it comprises one or more consensus sequence(s) comprising or consisting of the amino acid sequence K/R-(Z)₀₋₉-D/E-X-N-Y-S/T-(Z)₀₋₉-K/R (SEQ ID NO: 46) as defined above; for example one or more consensus sequence(s) having the amino acid sequence K/R-D/E-X₁-N-X₂-S/T-Z₁-Z₂-K/R (SEQ ID NO 19), wherein X₁ and X₂ are independently any amino acid apart from proline, and wherein Z₁ and Z₂. are not lysine or arginine. In an embodiment, said consensus sequence comprises or consists of the amino acid sequence of SEQ ID NO: 20. In an embodiment, said consensus sequence comprises or consists of the amino acid sequence of any one of SEQ ID Nos: 42-45. In a preferred embodiment, said consensus sequence comprises or consists of the amino acid sequence of any one of SEQ ID Nos: 42-44.

In an embodiment, said modified PspA protein comprises more than one said consensus sequence, for example 2, 3, 4 or 5 consensus sequences. In a preferred embodiment, wherein multiple consensus sequences are present, the consensus sequences have different sequences in order that glycosylation at each individual site may be distinguished.

In an embodiment, the modified PspA protein of the invention may be derived from an amino acid sequence at least 80%, 85%, 90%, 92%, 95%, 96%, 97%, 98% or 99% A identical to SEQ ID NO: 7 which is an immunogenic fragment and/or a variant of SEQ ID NO: 7. In an embodiment, the modified PspA protein of the invention may be derived from an immunogenic fragment of SEQ ID NO: 7 comprising at least about 15, at least about 20, at least about 40, or at least about 60 contiguous amino acid residues of the full length sequence, wherein said polypeptide is capable of eliciting an immune response specific for said amino acid sequence.

In an embodiment, the modified PspA protein of the invention may be derived from an amino acid sequence at least 80%, 85%, 90%, 92%, 95%, 96%, 97%, 98% or 99% A identical to SEQ ID NO: 7 which is a variant of SEQ ID NO: 7 which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, the modified PspA protein of the present invention may be derived from a variant in which 1 to 10, 5 to 10, 1 to 5, 1 to 3, 1 to 2 or 1 amino acid are substituted, deleted, or added in any combination.

In an embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated on the basis of the method described by Jameson and Wolf (CABIOS 4:181-186 [1988]).

The term “modified PspA protein” refers to a PspA amino acid sequence (for example, having a PspA amino acid sequence of SEQ ID NO: 7 or an amino acid sequence at least 80%, 85%, 90%, 91%, 92%, 93%, 94%, 95%, 96%, 97%, 98% or 99% identical to SEQ ID NO: 7), which PspA amino acid sequence may be a wild-type mature PspA amino acid sequence (for example, a wild-type amino acid sequence of SEQ ID NO: 7), which has been modified by the addition, substitution or deletion of one or more amino acids (for example, addition (insertion) of a consensus sequence(s) with amino acid sequence SEQ ID NO:19 or 46; or by substitution of one or more amino acids by a consensus sequence(s) with amino acid sequence SEQ ID NO:19 or 46. The modified PspA protein may also comprise further modifications (additions, substitutions, deletions) as well as the addition or substitution of one or more consensus sequence(s). For example, a signal sequence and/or peptide tag may be added. Additional amino acids at the N and/or C-terminal may be included to aid in cloning (for example, after the signal sequence or before the peptide tag, where present). In an embodiment, the modified PspA protein of the invention may be a non-naturally occurring PSPA protein.

In an embodiment of the invention, one or more amino acids (e.g. 1-7 amino acids, e.g. one amino acid) of the modified PspA amino acid sequence (for example, having an amino acid sequence of SEQ ID NO: 7 or a PSPA amino acid sequence at least 80%, 85%, 90%, 91%, 92%, 93%, 94%, 95%, 96%, 97%, 98% or 99% identical to SEQ ID NO: 7) have been substituted by a consensus sequence(s) with amino acid sequence SEQ ID NO:19, for example SEQ ID NO 20 or 42-45, in particular SEQ ID Nos: 42-44. For example, a single amino acid in the PspA amino acid sequence (e.g. SEQ ID NO: 7) may be replaced with a said consensus sequence. Alternatively, 2, 3, 4, 5, 6 or 7 amino acids in the PspA amino acid sequence (e.g. SEQ ID NO: 7 or a PspA amino acid sequence at least 80%, 85%, 90%, 92%, 95%, 96%, 97%, 98% or 99% identical to SEQ ID NO: 7) may be replaced with said consensus sequence.

Introduction of a consensus sequence(s) enables the modified PspA protein to be glycosylated. Thus, the present invention also provides a modified PspA protein of the invention wherein the modified PspA protein is glycosylated. In specific embodiments, the consensus sequences are introduced into specific regions of the PspA amino acid sequence, e.g. surface structures of the protein, at the N or C termini of the protein, and/or in loops that are stabilized by disulfide bridges.

A person skilled in the art will understand that when the PspA amino acid sequence is a variant and/or fragment of an amino acid sequence of SEQ ID NO: 7, such as an amino acid sequence at least 80%, 85%, 90%, 92%, 95%, 96%, 97%, 98% or 99% identical to SEQ ID NO: 7, the reference to “between amino acids . . . ” refers to the position that would be equivalent to the defined position, if this sequence was lined up with an amino acid sequence of SEQ ID NO: 7 in order to maximise the sequence identity between the two sequences. Sequence alignment tools are described above. The addition or deletion of amino acids from the variant and/or fragment of SEQ ID NO: 7 could lead to a difference in the actual amino acid position of the consensus sequence in the mutated sequence, however, by lining the mutated sequence up with the reference sequence, the amino acid in in an equivalent position to the corresponding amino acid in the reference sequence can be identified and hence the appropriate position for addition or substitution of the consensus sequence can be established.

Introduction of such glycosylation sites can be accomplished by, e.g. adding new amino acids to the primary structure of the protein (i.e. the glycosylation sites are added, in full or in part), or by mutating existing amino acids in the protein in order to generate the glycosylation sites (i.e. amino acids are not added to the protein, but selected amino acids of the protein are mutated so as to form glycosylation sites). Those of skill in the art will recognize that the amino acid sequence of a protein can be readily modified using approaches known in the art, e.g. recombinant approaches that include modification of the nucleic acid sequence encoding the protein.

In an embodiment, the modified PspA protein of the invention further comprises a “peptide tag” or “tag”, i.e. a sequence of amino acids that allows for the isolation and/or identification of the modified PspA protein. For example, adding a tag to a modified PspA protein of the invention can be useful in the purification of that protein and, hence, the purification of conjugate vaccines comprising the tagged modified PspA protein. Exemplary tags that can be used herein include, without limitation, histidine (HIS) tags. I one embodiment, the tag is a hexa-histidine tag. In certain embodiments, the tags used herein are removable, e.g. removal by chemical agents or by enzymatic means, once they are no longer needed, e.g. after the protein has been purified. Optionally the peptide tag is located at the C-terminus of the amino acid sequence. Optionally the peptide tag comprises six histidine residues at the C-terminus of the amino acid sequence. The peptide tag may comprise or be preceded by one, two or more additional amino acid residues, for example alanine, serine and/or glycine residues, e.g. GS.

### Signal Sequences and Other Modifications

In an embodiment, the modified carrier protein of the invention comprises a signal sequence which is capable of directing the protein to the periplasm of a host cell (e.g. bacterium). In a specific embodiment, the signal sequence is from S. flexneri flagellin (FlgI) [MIKFLSALILLLVTTAAQA (SEQ ID NO: 21)]. In other embodiments, the signal sequence is from E. coli outer membrane porin A (OmpA) [MKKTAIAIAVALAGFATVAQA (SEQ ID NO: 22)], E. coli maltose binding protein (MalE) [MKIKTGARILALSALTTMMFSASALA (SEQ ID NO: 23)], Pectobacterium carotovorum pectate lyase (PelB) [MKYLLPTAAAGLLLLAAQPAMA (SEQ ID NO: 24], heat labile E. coli enterotoxin LTIIb [MSFKKIIKAFVIMAALVSVQAHA (SEQ ID NO: 25)], Bacillus subtilis endoxylanase XynA [MFKFKKKFLVGLTAAFMSISMFSATASA (SEQ ID NO: 26)], E. coli DsbA [MKKIWLALAGLVLAFSASA (SEQ ID NO: 27)], TolB [MKQALRVAFGFLILWASVLHA (SEQ ID NO: 28)] or S. agalactiae SipA [MKMNKKVLLTSTMAASLLSVASVQAS (SEQ ID NO: 29)]. In an embodiment, the signal sequence has an amino acid sequence at least 80%, 85%, 90%, 92%, 95%, 96%, 97%, 98%, 99% or 100% identical to a SEQ ID NO: 21-29. In one aspect, the signal sequence has an amino acid sequence at least 80%, 85%, 90%, 95%, 96%, 97%, 98%, 99% or 100% identical to E. coli flagellin signal sequence (FlgI) [MIKFLSALILLLVTTAAQA (SEQ ID NO: 21)].

In an embodiment, a serine and/or alanine residue is added between the signal sequence and the start of the sequence of the mature protein, e.g. SA or S, preferably S. Such a reside or residues have the advantage of leading to more efficient cleavage of the leader sequence.

### Glycosylation Sites

The invention provides novel universal PglB specific consensus sequences for glycosylation sites compatible with the quantification of glycosylation site occupancy by LC-MS. The present inventors determined several features that would be shared by such sequences and thus by the consensus sequences of the invention:

Generate tryptic peptides that are between 8 and 16 amino acids in length, e.g. 8, 9, 10, 11, 12, 13, 14 15 or 16.

Show a strong and reproducible signal in mass spectrometry analysis (parental and transition ions);

Commence and terminate with an arginine or lysine (for trypsin cleavage) and do not contain a cysteine (to increase the ionization capability of the tryptic peptide);

Preferably does not contain amino acids susceptible to modification (asparagine and glutamine amino acid residues which are susceptible to deamination, or methionine, cysteine and tryptophan amino acid residues which are susceptible to oxidation or hydrophobic or aromatic amino acids);

Be localized on well exposed loops on the protein surface in order to be accessible to oligosaccharyltransferase enzyme (PglB) also in an at least partially folded molecule and do not interfere with normal process of folding.

Thus, the invention provides a consensus sequence comprising or consisting of the following amino acid sequence:

K/R-Z₀₋₉-D/E-X-N-Y-S/T-Z₀₋₉-K/R
wherein X and Y are independently any amino acid except proline, and Z represents any amino acid. In a preferred embodiment, X and Y are independently any amino acid except proline, lysine or arginine. In an embodiment, Z represents any amino acid except lysine or arginine. In an embodiment, X, Y and/or Z are not aromatic or hydrophobic amino acids. In a preferred embodiment, Z represents any amino acid except cysteine, methionine, asparagine, glutamine, lysine or arginine (eg SEQ ID NO: 47).

Preferably, the total length of said consensus sequence is 16 or fewer amino acids, for example 8, 10, 11, 12, 13, 14, 15 or 16 amino acids. Preferably, the total length of the sequence is 8 or more amino acids, for example 8, 10, 11, 12, 13, 14, 15 or 16 amino acids.

In a specific embodiment, the invention provides a consensus sequence(s) comprising or consisting of the amino acid sequence K/R-D/E-X₁-N-X₂-S/T-Z₁-Z₂-K/R (SEQ ID NO 19), wherein X₁ and X₂ are independently any amino acid apart from proline, lysine or arginine and wherein Z₁ and Z₂. are not lysine or arginine or cysteine. In an embodiment, the consensus sequence comprises or consists of the amino acid sequence of SEQ ID NO: 20. In an embodiment, the consensus sequence comprises or consists of the amino acid sequence of any one of SEQ ID No: 42, SEQ ID No: 43; SEQ ID No: 44 or SEQ ID No 45, preferably SEQ ID Nos: 42-44.

### Polysaccharides

In an embodiment, one of the antigens in a conjugate (e.g. bioconjugate) of the invention is a saccharide such as a bacterial capsular saccharide, a bacterial lipopolysaccharide or a bacterial oligosaccharide. In an embodiment the antigen is a bacterial capsular saccharide.

The saccharides may be selected from a group consisting of: Staphylococcus aureus type 5 capsular saccharide, Staphylococcus aureus type 8 capsular saccharide, N. meningitidis serogroup A capsular saccharide (MenA), N. meningitidis serogroup C capsular saccharide (MenC), N. meningitidis serogroup Y capsular saccharide (MenY), N. meningitidis serogroup W capsular saccharide (MenW), H. influenzae type b capsular saccharide (Hib), Group B Streptococcus group I capsular saccharide, Group B Streptococcus group II capsular saccharide, Group B Streptococcus group III capsular saccharide, Group B Streptococcus group IV capsular saccharide, Group B Streptococcus group V capsular saccharide, Vi saccharide from Salmonella typhi, N. meningitidis LPS (such as L3 and/or L2), M. catarrhalis LPS, H. influenzae LPS, Shigella O-antigens, P. aeruginosa O-antigens, E. coli O-antigens or S. pneumoniae capsular polysaccharide.

In an embodiment, the antigen is a polysaccharide or oligosaccharide. In an embodiment, the antigen comprises two or more monosaccharides, for example 2, 3, 4, 5, 6, 7, 8, 9, 10 or more monosaccharides. In an embodiment, the antigen is an oligosaccharide containing no more than 20, 15, 12, 10, 9, or 8 monosaccharides. In an embodiment, the antigen is an oligosaccharide containing no more than no more than 500, 400, 300, 200, 100, 90, 80, 70, 60, 50, 40, 30, 20, 10 or 5 monosaccharides.

### Host Cell

The present invention also provides a host cell comprising:

i) one or more nucleic acids that encode glycosyltransferase(s);
ii) a nucleic acid that encodes an oligosaccharyl transferase;
iii) a nucleic acid that encodes a modified carrier protein of the invention; and optionally
iv) a nucleic acid that encodes a polymerase (e.g. wzy).

Host cells that can be used to produce the bioconjugates of the invention, include archea, prokaryotic host cells, and eukaryotic host cells. Exemplary prokaryotic host cells for use in production of the bioconjugates of the invention, without limitation, Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species, Bacillus species, and Clostridium species. In a specific embodiment, the host cell is E. coli.

In an embodiment, the host cells used to produce the bioconjugates of the invention are engineered to comprise heterologous nucleic acids, e.g. heterologous nucleic acids that encode one or more carrier proteins and/or heterologous nucleic acids that encode one or more proteins, e.g. genes encoding one or more proteins. In a specific embodiment, heterologous nucleic acids that encode proteins involved in glycosylation pathways (e.g. prokaryotic and/or eukaryotic glycosylation pathways) may be introduced into the host cells of the invention. Such nucleic acids may encode proteins including, without limitation, oligosaccharyl transferases, epimerases, flippases, polymerases, and/or glycosyltransferases. Heterologous nucleic acids (e.g. nucleic acids that encode carrier proteins and/or nucleic acids that encode other proteins, e.g. proteins involved in glycosylation) can be introduced into the host cells of the invention using methods such as electroporation, chemical transformation by heat shock, natural transformation, phage transduction, and conjugation. In specific embodiments, heterologous nucleic acids are introduced into the host cells of the invention using a plasmid, e.g. the heterologous nucleic acids are expressed in the host cells by a plasmid (e.g. an expression vector). In another specific embodiment, heterologous nucleic acids are introduced into the host cells of the invention using the method of insertion described in International Patent application No. PCT/EP2013/068737 (published as WO 14/037585).

Thus, the present invention also provides a host cell comprising:

i) one or more nucleic acids that encode glycosyltransferase(s);
ii) a nucleic acid that encodes an oligosaccharyl transferase;
iii) a nucleic acid that encodes a modified carrier protein of the invention;
iv) a nucleic acid that encodes a polymerase (e.g. wzy); and a nucleic acid that encodes a flippase (e.g. wxy).

In an embodiment, additional modifications may be introduced (e.g. using recombinant techniques) into the host cells of the invention. For example, host cell nucleic acids (e.g. genes) that encode proteins that form part of a possibly competing or interfering glycosylation pathway (e.g. compete or interfere with one or more heterologous genes involved in glycosylation that are recombinantly introduced into the host cell) can be deleted or modified in the host cell background (genome) in a manner that makes them inactive/dysfunctional (i.e. the host cell nucleic acids that are deleted/modified do not encode a functional protein or do not encode a protein whatsoever). In an embodiment, when nucleic acids are deleted from the genome of the host cells of the invention, they are replaced by a desirable sequence, e.g. a sequence that is useful for glycoprotein production.

Exemplary genes that can be deleted in host cells (and, in some cases, replaced with other desired nucleic acid sequences) include genes of host cells involved in glycolipid biosynthesis, such as waaL (see, e.g. Feldman et al. 2005, PNAS USA 102:3016-3021), the lipid A core biosynthesis cluster (waa), galactose cluster (gal), arabinose cluster (ara), colonic acid cluster (wc), capsular polysaccharide cluster, undecaprenol-pyrophosphate biosynthesis genes (e.g. uppS (Undecaprenyl pyrophosphate synthase), uppP (Undecaprenyl diphosphatase)), Und-P recycling genes, metabolic enzymes involved in nucleotide activated sugar biosynthesis, enterobacterial common antigen cluster, and prophage O antigen modification clusters like the gtrABS cluster.

Such a modified prokaryotic host cell comprises nucleic acids encoding enzymes capable of producing a bioconjugate comprising an antigen, for example a saccharide antigen attached to a modified Hla carrier protein of the invention. Such host cells may naturally express nucleic acids specific for production of a saccharide antigen, or the host cells may be made to express such nucleic acids, i.e. in certain embodiments said nucleic acids are heterologous to the host cells. In certain embodiments, one or more of said nucleic acids specific for production of a saccharide antigen are heterologous to the host cell and integrated into the genome of the host cell. In certain embodiments, the host cells of the invention comprise nucleic acids encoding additional enzymes active in the N-glycosylation of proteins, e.g. the host cells of the invention further comprise a nucleic acid encoding an oligosaccharyl transferase and/or one or more nucleic acids encoding other glycosyltransferases.

Nucleic acid sequences comprising capsular polysaccharide gene clusters can be inserted into the host cells of the invention. In a specific embodiment, the capsular polysaccharide gene cluster inserted into a host cell of the invention is a capsular polysaccharide gene cluster from an E. coli strain, a Staphylococcus strain (e.g. S. aureus), a Streptococcus strain (e.g. S. pneumoniae, S. pyrogenes, S. agalacticae), or a Burkholderia strain (e.g. B mallei, B. pseudomallei, B. thailandensis). Disclosures of methods for making such host cells which are capable of producing bioconjugates are found in WO 06/119987, WO 09/104074, WO 11/62615, WO 11/138361, WO 14/57109, WO14/72405 and WO16/20499.

In an embodiment, the host cell comprises a nucleic acid that encodes a modified carrier protein of the invention in a plasmid in the host cell.

### Glycosylation Machinery

The host cells of the invention comprise, and/or can be modified to comprise, nucleic acids that encode genetic machinery (e.g. glycosyltransferases, flippases, polymerases, and/or oligosaccharyltransferases) capable of producing hybrid oligosaccharides and/or polysaccharides, as well as genetic machinery capable of linking antigens to the modified carrier proteins of the invention.

Capsular polysaccharides are assembled on the bacterial membrane carrier lipid undecaprenyl pyrophosphate by a conserved pathway that shares homology to the polymerase-dependent pathway of O polysaccharide synthesis in Gram-negative bacteria. O antigen assembly is initiated by the transfer of a sugar phosphate from a DP-donor to undecaprenyl phosphate. The lipid linked O antigen is assembled at the cytoplasmic side of the inner membrane by sequential action of different glycosyltransferases. The glycolipid is then flipped to the periplasmic space and polymerised. By replacing the O antigen ligase WaaL with the oligosaccharyltransferase PglB, the polymerised O antigen can be transferred to a protein carrier rather than to the lipid A core.

### Glycosyltransferases

The host cells of the invention comprise nucleic acids that encode glycosyltransferases that produce an oligosaccharide or polysaccharide repeat unit. In an embodiment, said repeat unit does not comprise a hexose at the reducing end, and said oligosaccharide or polysaccharide repeat unit is derived from a donor oligosaccharide or polysaccharide repeat unit that comprises a hexose at the reducing end.

In an embodiment, the host cells of the invention may comprise a nucleic acid that encodes a glycosyltransferase that assembles a hexose monosaccharide derivative onto undecaprenyl pyrophosphate (Und-PP). In one aspect, the glycosyltransferase that assembles a hexose monosaccharide derivative onto Und-PP is heterologous to the host cell and/or heterologous to one or more of the genes that encode glycosyltransferase(s). Said glycosyltransferase can be derived from, e.g. Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species. In a specific embodiment, the glycosyltransferase that assembles a hexose monosaccharide derivative onto Und-PP is wecA, optionally from E. coli (wecA can assemble GlcNAc onto UndP from UDP-GlcNAc). In an embodiment, the hexose monosaccharide is selected from the group consisting of glucose, galactose, rhamnose, arabinotol, fucose and mannose (e.g. galactose).

In an embodiment, the host cells of the invention may comprise nucleic acids that encode one or more glycosyltransferases capable of adding a monosaccharide to the hexose monosaccharide derivative assembled on Und-PP. In a specific embodiment, said one or more glycosyltransferases capable of adding a monosaccharide to the hexose monosaccharide derivative is the galactosyltransferase (wfeD) from Shigella boyedii. In another specific embodiment, said one or more glycosyltransferases capable of adding a monosaccharide to the hexose monosaccharide derivative is the galactofuranosyltransferase (wbeY) from E. coli O28. In another specific embodiment, said one or more glycosyltransferases capable of adding a monosaccharide to the hexose monosaccharide derivative is the galactofuranosyltransferase (wfdK) from E. coli O167. Galf-transferases, such as wfdK and wbeY, can transfer Galf (Galactofuranose) from UDP-Galf to -GlcNAc-P-P-Undecaprenyl. In another specific embodiment, said one or more glycosyltransferases capable of adding a monosaccharide to the hexose monosaccharide derivative are the galactofuranosyltransferase (wbeY) from E. coli O28 and the galactofuranosyltransferase (wfdK) from E. coli O167.

In an embodiment, the host cells of the invention comprise nucleic acids that encode glycosyltransferases that assemble the donor oligosaccharide or polysaccharide repeat unit onto the hexose monosaccharide derivative.

In an embodiment, the glycosyltransferases that assemble the donor oligosaccharide or polysaccharide repeat unit onto the hexose monosaccharide derivative comprise a glycosyltransferase that is capable of adding the hexose monosaccharide present at the reducing end of the first repeat unit of the donor oligosaccharide or polysaccharide to the hexose monosaccharide derivative. Exemplary glycosyltransferases include galactosyltransferases (wciP), e.g. wciP from E. coli O21.

In one embodiment, the glycosyltransferases that assemble the donor oligosaccharide or polysaccharide repeat unit onto the hexose monosaccharide derivative comprise a glycosyltransferase that is capable of adding the monosaccharide that is adjacent to the hexose monosaccharide present at the reducing end of the first repeat unit of the donor oligosaccharide or polysaccharide to the hexose monosaccharide present at the reducing end of the first repeat unit of the donor oligosaccharide or polysaccharide. Exemplary glycosyltransferases include glucosyltransferase (wciQ), e.g. wciQ from E. coli O21.

In an embodiment, a host cell of the invention comprises glycosyltransferases for synthesis of the repeat units of an oligosaccharide or polysaccharide selected from the Staphylococcus aureus CP5 or CP8 gene cluster. In a specific embodiment, the glycosyltransferases for synthesis of the repeat units of an oligosaccharide or polysaccharide are from the Staphylococcus aureus CP5 gene cluster. S. aureus CP5 and CP8 have a similar structure to P. aeruginosa O11 antigen synthetic genes, so these genes may be combined with E. coli monosaccharide synthesis genes to synthesise an undecaprenyl pyrophosphate-linked CP5 or CP8 polymer consisting of repeating trisaccharide units.

In an embodiment, a host cell of the invention comprises glycosyltransferases that assemble the donor oligosaccharide or polysaccharide repeat unit onto the hexose monosaccharide derivative comprise a glycosyltransferase that is capable of adding the hexose monosaccharide present at the reducing end of the first repeat unit of the donor oligosaccharide or polysaccharide to the hexose monosaccharide derivative.

### Oligosaccharyl Transferases

N-linked protein glycosylation—the addition of carbohydrate molecules to an asparagine residue in the polypeptide chain of the target protein—is the most common type of post-translational modification occurring in the endoplasmic reticulum of eukaryotic organisms. The process is accomplished by the enzymatic oligosaccharyltransferase complex (OST) responsible for the transfer of a preassembled oligosaccharide from a lipid carrier (dolichol phosphate) to an asparagine residue of a nascent protein within the conserved sequence Asn-X-Ser/Thr (where X is any amino acid except proline) in the Endoplasmic reticulum.

It has been shown that a bacterium, the food-borne pathogen Campylobacter jejuni, can also N-glycosylate its proteins (Wacker et al. Science. 2002; 298(5599):1790-3) due to the fact that it possesses its own glycosylation machinery. The machinery responsible of this reaction is encoded by a cluster called “pgl” (for protein glycosylation).

The C. jejuni glycosylation machinery can be transferred to E. coli to allow for the glycosylation of recombinant proteins expressed by the E. coli cells. Previous studies have demonstrated how to generate E. coli strains that can perform N-glycosylation (see, e.g. Wacker et al. Science. 2002; 298 (5599):1790-3; Nita-Lazar et al. Glycobiology. 2005; 15(4):361-7; Feldman et al. Proc Natl Acad Sci USA. 2005; 102(8):3016-21; Kowarik et al. EMBO J. 2006; 25(9):1957-66; Wacker et al. Proc Natl Acad Sci USA. 2006; 103(18):7088-93; International Patent Application Publication Nos. WO2003/074687, WO2006/119987, WO 2009/104074, and WO/2011/06261, and WO2011/138361).PglB mutants having optimised properties are described in WO2016/107818. A preferred mutant is PglB_{cuo N311V-K482R-D483H-A669V}.

Oligosaccharyl transferases transfer lipid-linked oligosaccharides to asparagine residues of nascent polypeptide chains that comprise a N-glycosylation consensus motif, e.g. Asn-X-Ser(Thr), wherein X can be any amino acid except Pro; or Asp(Glu)-X-Asn-Z-Ser(Thr), wherein X and Z are independently selected from any natural amino acid except Pro (see WO 2006/119987). See, e.g. WO 2003/074687 and WO 2006/119987, the disclosures of which are herein incorporated by reference in their entirety.

In an embodiment, the host cells of the invention comprise a nucleic acid that encodes an oligosaccharyl transferase. The nucleic acid that encodes an oligosaccharyl transferase can be native to the host cell or can be introduced into the host cell using genetic approaches, as described above. In a specific embodiment, the oligosaccharyl transferase is an oligosaccharyl transferase from Campylobacter. In another specific embodiment, the oligosaccharyl transferase is an oligosaccharyl transferase from Campylobacter jejuni (i.e. pglB; see, e.g. Wacker et al. 2002, Science 298:1790-1793; see also, e.g. NCBI Gene ID: 3231775, UniProt Accession No. 086154). In another specific embodiment, the oligosaccharyl transferase is an oligosaccharyl transferase from Campylobacter lari (see, e.g. NCBI Gene ID: 7410986).

In a specific embodiment, the host cells of the invention comprise a nucleic acid sequence encoding an oligosaccharyl transferase, wherein said nucleic acid sequence encoding an oligosaccharyl transferase (e.g. pglB from Campylobacter jejuni) is integrated into the genome of the host cell.

In a specific embodiment, the host cells of the invention comprise a nucleic acid sequence encoding an oligosaccharyl transferase, wherein said nucleic acid sequence encoding an oligosaccharyl transferase (e.g. pglB from Campylobacter jejuni) is plasmid-borne.

In another specific embodiment, provided herein is a modified prokaryotic host cell comprising (i) a glycosyltransferase derived from an capsular polysaccharide cluster from S. aureus, wherein said glycosyltransferase is integrated into the genome of said host cell; (ii) a nucleic acid encoding an oligosaccharyl transferase (e.g. pglB from Campylobacter jejuni), wherein said nucleic acid encoding an oligosaccharyl transferase is plasmid-borne and/or integrated into the genome of the host cell; and (iii) a modified carrier protein of the invention, wherein said modified carrier protein is either plasmid-borne or integrated into the genome of the host cell. There is also provided a method of making a modified prokaryotic host cell comprising (i) integrating a glycosyltransferase derived from an capsular polysaccharide cluster into the genome of said host cell; (ii) integrating into the host cell one or more nucleic acids encoding an oligosaccharyl transferase (e.g. pglB from Campylobacter jejuni) which is plasmid-borne and/or integrated into the genome of the host cell; and (iii) integrating into a host cell a modified carrier protein of the invention either plasmid-borne or integrated into the genome of the host cell.

In specific embodiment is a host cell of the invention, wherein at least one gene of the host cell has been functionally inactivated or deleted, optionally wherein the waaL gene of the host cell has been functionally inactivated or deleted, optionally wherein the waaL gene of the host cell has been replaced by a nucleic acid encoding an oligosaccharyltransferase, optionally wherein the waaL gene of the host cell has been replaced by C. jejuni pglB.

### Polymerases

In an embodiment, a polymerase (e.g. wzy) is introduced into a host cell of the invention (i.e. the polymerase is heterologous to the host cell). In an embodiment, the polymerase is a bacterial polymerase. In an embodiment, the polymerase is a capsular polysaccharide polymerase (e.g. wzy) or an O antigen polymerase (e.g. wzy). In an embodiment, the polymerase is a capsular polysaccharide polymerase (e.g. wzy).

In an embodiment, a polymerase of a capsular polysaccharide biosynthetic pathway is introduced into a host cell of the invention.

### Flippases

In an embodiment, a flippase (wzx or homologue) is introduced into a host cell of the invention (i.e. the flippase is heterologous to the host cell). Thus, a host cell of the invention may further comprise a flippase. In an embodiment, the flippase is a bacterial flippase. Flippases translocate wild type repeating units and/or their corresponding engineered (hybrid) repeat units from the cytoplasm into the periplam of host cells (e.g. E. coli). Thus, a host cell of the invention may comprise a nucleic acid that encodes a flippase (wzx).

In a specific embodiment, a flippase of a capsular polysaccharide biosynthetic pathway is introduced into a host cell of the invention.

### Genetic Background

Exemplary host cells that can be used to generate the host cells of the invention include, without limitation, Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species, Bacillus species, and Clostridium species. In a specific embodiment, the host cell used herein is E. coli.

In an embodiment, the host cell genetic background is modified by, e.g. deletion of one or more genes. Exemplary genes that can be deleted in host cells (and, in some cases, replaced with other desired nucleic acid sequences) include genes of host cells involved in glycolipid biosynthesis, such as waaL (see, e.g. Feldman et al. 2005, PNAS USA 102:3016-3021), the O antigen cluster (rfb or wb), enterobacterial common antigen cluster (wec), the lipid A core biosynthesis cluster (waa), and prophage O antigen modification clusters like the gtrABS cluster. In a specific embodiment, one or more of the waaL gene, gtrA gene, gtrB gene, gtrS gene, or a gene or genes from the wec cluster or a gene or genes from the rfb gene cluster are deleted or functionally inactivated from the genome of a prokaryotic host cell of the invention. In one embodiment, a host cell used herein is E. coli, wherein the waaL gene, gtrA gene, gtrB gene, gtrS gene are deleted or functionally inactivated from the genome of the host cell. In another embodiment, a host cell used herein is E. coli, wherein the waaL gene and gtrS gene are deleted or functionally inactivated from the genome of the host cell. In another embodiment, a host cell used herein is E. coli, wherein the waaL gene and genes from the wec cluster are deleted or functionally inactivated from the genome of the host cell.

### Bioconjugates

The host cells of the invention can be used to produce bioconjugates comprising a saccharide antigen, for example a bacterial capsular polysaccharide antigen linked to a modified carrier protein of the invention. In an embodiment, the polysaccharide is linked to asparagine in the modified carrier protein, for example via N-acetylglucosamine. Methods of producing bioconjugates using host cells are described for example in WO 2003/074687, WO 2006/119987 and WO2011/138361. Bioconjugates, as described herein, have advantageous properties over chemical conjugates of antigen-carrier protein, in that they require less chemicals in manufacture and are more consistent in terms of the final product generated.

In an embodiment, provided herein is a bioconjugate comprising a modified carrier protein of the invention linked to a polysaccharide, in particular a polysaccharide antigen. In an embodiment, provided herein is a bioconjugate comprising a modified carrier protein of the invention and an antigen selected from Staphylococcus aureus type 5 capsular saccharide, Staphylococcus aureus type 8 capsular saccharide, N. meningitidis serogroup A capsular saccharide (MenA), N. meningitidis serogroup C capsular saccharide (MenC), N. meningitidis serogroup Y capsular saccharide (MenY), N. meningitidis serogroup W capsular saccharide (MenW), H. influenzae type b capsular saccharide (Hib), Group B Streptococcus group I capsular saccharide, Group B Streptococcus group II capsular saccharide, Group B Streptococcus group III capsular saccharide, Group B Streptococcus group IV capsular saccharide, Group B Streptococcus group V capsular saccharide, Vi saccharide from Salmonella typhi, N. meningitidis LPS (such as L3 and/or L2), M. catarrhalis LPS, H. influenzae LPS, Shigella O-antigens, P. aeruginosa O-antigens, E. coli O-antigens or S. pneumoniae capsular polysaccharide.

The bioconjugates of the invention can be purified for example, by chromatography (e.g. ion exchange, cationic exchange, anionic exchange, affinity, and sizing column chromatography), centrifugation, differential solubility, or by any other standard technique for the purification of proteins. See, e.g. Saraswat et al. 2013, Biomed. Res. Int. ID #312709 (p. 1-18); see also the methods described in WO 2009/104074. Further, the bioconjugates may be fused to heterologous polypeptide sequences described herein or otherwise known in the art to facilitate purification. The actual conditions used to purify a particular bioconjugate will depend, in part, on the synthesis strategy and on factors such as net charge, hydrophobicity, and/or hydrophilicity of the bioconjugate, and will be apparent to those having skill in the art.

A further aspect of the invention is a process for producing a bioconjugate that comprises (or consists of) a modified carrier protein linked to a polysaccharide, said method comprising (i) culturing the host cell of the invention under conditions suitable for the production of proteins (and optionally under conditions suitable for the production of saccharides) and (ii) isolating the bioconjugate produced by said host cell.

A further aspect of the invention is a bioconjugate produced by the process of the invention, wherein said bioconjugate comprises a saccharide linked to a modified carrier protein.

### Mass Spectrometry Methods

The present invention provides carrier proteins by analytics driven design approach that allows measurement of the glycosylation site occupancy by liquid chromatography coupled to mass spectrometry (LC-MS). This is particularly relevant to (i) quantify the unglycosylated carrier in the final product, (ii) follow in process the rate of bioconjugation and (iii) quantify the extent of glycosylation on single sites, in the case of carrier proteins designed with multiple sites for glycosylation, to increase the rate of glycosylation.

The strategy is based on the quantification of the natively unglycosylated form of the glycopeptide, using isotopically labeled internal standards. In particular, two sets of heavy isotope labeled peptide standards are spiked into the sample before proteolysis, and the digested sample is analyzed by LC-MS. One set of peptide standards is employed to determine the total glycoprotein amount, while the other standard monitors the unglycosylated amount of the glycoprotein. In this way, the abundance of the glycosylated portion of the protein is calculated by subtracting the unglycosylated protein amount from the total protein amount, and the site occupancy is then determined.

### Immunogenic Compositions

The modified carrier proteins and conjugates (e.g. bioconjugates), of the invention are particularly suited for inclusion in immunogenic compositions and vaccines. The present invention provides an immunogenic composition comprising a modified carrier protein of the invention, or the conjugate of the invention, or the bioconjugate of the invention.

Also provided is a method of making the immunogenic composition of the invention comprising the step of mixing the modified carrier protein or the conjugate (e.g. bioconjugate) of the invention with a pharmaceutically acceptable excipient or carrier.

Immunogenic compositions comprise an immunologically effective amount of the modified carrier protein or conjugate (e.g. bioconjugate) of the invention, as well as any other components. By “immunologically effective amount”, it is meant that the administration of that amount to an individual, either as a single dose or as part of a series is effective for treatment or prevention. This amount varies depending on the health and physical condition of the individual to be treated, age, the degree of protection desired, the formulation of the vaccine and other relevant factors. It is expected that the amount will fall in a relatively broad range that can be determined through routine trials.

Immunogenic compositions if the invention may also contain diluents such as water, saline, glycerol etc. Additionally, auxiliary substances, such as wetting or emulsifying agents, pH buffering substances, polyols and the like may be present.

The immunogenic compositions comprising the modified carrier protein of the invention or conjugates (or bioconjugates) may comprise any additional components suitable for use in pharmaceutical administration. In specific embodiments, the immunogenic compositions of the invention are monovalent formulations. In other embodiments, the immunogenic compositions of the invention are multivalent formulations, e.g. bivalent, trivalent, and tetravalent formulations. For example, a multivalent formulation comprises more than one antigen for example more than one conjugate.

### Vaccines

The present invention also provides a vaccine comprising an immunogenic composition of the invention and a pharmaceutically acceptable excipient or carrier.

Pharmaceutically acceptable excipients and carriers can be selected by those of skill in the art. For example, the pharmaceutically acceptable excipient or carrier can include a buffer, such as Tris (trimethamine), phosphate (e.g. sodium phosphate), acetate, borate (e.g. sodium borate), citrate, glycine, histidine and succinate (e.g. sodium succinate), suitably sodium chloride, histidine, sodium phosphate or sodium succinate. The pharmaceutically acceptable excipient may include a salt, for example sodium chloride, potassium chloride or magnesium chloride. Optionally, the pharmaceutically acceptable excipient contains at least one component that stabilizes solubility and/or stability. Examples of solubilizing/stabilizing agents include detergents, for example, laurel sarcosine and/or polysorbate (e.g. TWEEN™ 80). Examples of stabilizing agents also include poloxamer (e.g. poloxamer 124, poloxamer 188, poloxamer 237, poloxamer 338 and poloxamer 407). The pharmaceutically acceptable excipient may include a non-ionic surfactant, for example polyoxyethylene sorbitan fatty acid esters, Polysorbate-80 (TWEEN™ 80), Polysorbate-60 (TWEEN™ 60), Polysorbate-40 (TWEEN™ 40) and Polysorbate-20 (TWEEN™ 20), or polyoxyethylene alkyl ethers (suitably polysorbate-80). Alternative solubilizing/stabilizing agents include arginine, and glass forming polyols (such as sucrose, trehalose and the like). The pharmaceutically excipient may be a preservative, for example phenol, 2-phenoxyethanol, or thiomersal. Other pharmaceutically acceptable excipients include sugars (e.g. lactose, sucrose), and proteins (e.g. gelatine and albumin). Pharmaceutically acceptable carriers include water, saline solutions, aqueous dextrose and glycerol solutions. Numerous pharmaceutically acceptable excipients and carriers are described, for example, in Remington's Pharmaceutical Sciences, by E. W. Martin, Mack Publishing Co. Easton, Pa., 5th Edition (975).

In an embodiment, the immunogenic composition or vaccine of the invention additionally comprises one or more buffers, e.g. phosphate buffer and/or sucrose phosphate glutamate buffer. In other embodiments, the immunogenic composition or vaccine of the invention does not comprise a buffer.

In an embodiment, the immunogenic composition or vaccine of the invention additionally comprises one or more salts, e.g. sodium chloride, calcium chloride, sodium phosphate, monosodium glutamate, and aluminum salts (e.g. aluminum hydroxide, aluminum phosphate, alum (potassium aluminum sulfate), or a mixture of such aluminum salts). In other embodiments, the immunogenic composition or vaccine of the invention does not comprise a salt.

The immunogenic composition or vaccine of the invention may additionally comprise a preservative, e.g. a mercury derivative thimerosal. In a specific embodiment, the immunogenic composition or vaccine of the invention comprises 0.001% to 0.01% thimerosal. In other embodiments, the immunogenic composition or vaccine of the invention does not comprise a preservative.

The vaccine or immunogenic composition of the invention may also comprise an antimicrobial, typically when package in multiple dose format. For example, the immunogenic composition or vaccine of the invention may comprise 2-phenoxyethanol.

The vaccine or immunogenic composition of the invention may also comprise a detergent e.g. polysorbate, such as TWEEN™ 80. Detergents are generally present at low levels e.g. <0.01%, but higher levels have been suggested for stabilising antigen formulations e.g. up to 10%.

The immunogenic compositions of the invention can be included in a container, pack, or dispenser together with instructions for administration.

The immunogenic compositions or vaccines of the invention can be stored before use, e.g. the compositions can be stored frozen (e.g. at about −20° C. or at about −70° C.); stored in refrigerated conditions (e.g. at about 4° C.); or stored at room temperature.

The immunogenic compositions or vaccines of the invention may be stored in solution or lyophilized. In an embodiment, the solution is lyophilized in the presence of a sugar such as sucrose, trehalose or lactose. In another embodiment, the vaccines of the invention are lyophilized and extemporaneously reconstituted prior to use.

Vaccine preparation is generally described in Vaccine Design (“The subunit and adjuvant approach” (eds Powell M. F. & Newman M. J.) (1995) Plenum Press New York). Encapsulation within liposomes is described by Fullerton, U.S. Pat. No. 4,235,877.

The present invention also provides a vaccine comprising an immunogenic composition of the invention and a pharmaceutically acceptable excipient or carrier.

### Adjuvants

In an embodiment, the immunogenic compositions or vaccines of the invention comprise, or are administered in combination with, an adjuvant. The adjuvant for administration in combination with an immunogenic composition or vaccine of the invention may be administered before, concomitantly with, or after administration of said immunogenic composition or vaccine. In some embodiments, the term “adjuvant” refers to a compound that when administered in conjunction with or as part of an immunogenic composition of vaccine of the invention augments, enhances and/or boosts the immune response to a bioconjugate, but when the compound is administered alone does not generate an immune response to the modified carrier protein/conjugate/bioconjugate. In some embodiments, the adjuvant generates an immune response to the modified carrier protein, conjugate or bioconjugate and does not produce an allergy or other adverse reaction.

In an embodiment, the immunogenic composition or vaccine of the invention is adjuvanted. Adjuvants can enhance an immune response by several mechanisms including, e.g. lymphocyte recruitment, stimulation of B and/or T cells, and stimulation of macrophages. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see United Kingdom Patent GB2220211), MF59 (Novartis), AS03 (GlaxoSmithKline), AS04 (GlaxoSmithKline), polysorbate 80 (TWEEN™ 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al. in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al. N. Engl. J. Med. 336, 86-91 (1997)). Another adjuvant is CpG (Bioworld Today, Nov. 15, 1998).

In one aspect of the invention, the adjuvant is an aluminium salt such as aluminium hydroxide gel (alum) or aluminium phosphate.

In another aspect of the invention, the adjuvant is selected to be a preferential inducer of either a TH1 or a TH2 type of response. High levels of Th1-type cytokines tend to favor the induction of cell mediated immune responses to a given antigen, whilst high levels of Th2-type cytokines tend to favour the induction of humoral immune responses to the antigen. It is important to remember that the distinction of Th1 and Th2-type immune response is not absolute. In reality an individual will support an immune response which is described as being predominantly Th1 or predominantly Th2. However, it is often convenient to consider the families of cytokines in terms of that described in murine CD4+ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p 145-173). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of 11-4, IL-5, IL-6, IL-10. Suitable adjuvant systems which promote a predominantly Th1 response include: Monophosphoryl lipid A or a derivative thereof, particularly 3-de-O-acylated monophosphoryl lipid A (3D-MPL) (for its preparation see GB 2220211 A); MPL, e.g. 3D-MPL and the saponin QS21 in a liposome, for example a liposome comprising cholesterol and DPOC; and a combination of monophosphoryl lipid A, for example 3-de-O-acylated monophosphoryl lipid A, together with either an Aluminium salt (for instance Aluminium phosphate or Aluminium hydroxide) or an oil-in-water emulsion. In such combinations, the antigen and 3D-MPL may be contained in the same particulate structures, allowing for more efficient delivery of antigenic and immunostimulatory signals. Studies have shown that 3D-MPL is able to further enhance the immunogenicity of an Alum-adsorbed antigen (Thoelen et al. Vaccine (1998) 16:708-14; EP 689454-B1). Unmethylated CpG containing oligonucleotides (WO 96/02555) are also preferential inducers of a TH1 response and are suitable for use in the present invention.

The vaccine or immunogenic composition of the invention may contain an oil in water emulsion, since these have been suggested to be useful as adjuvant compositions (EP 399843; WO 95/17210). Oil in water emulsions such as those described in WO95/17210 (which discloses oil in water emulsions comprising from 2 to 10% squalene, from 2 to 10% alpha tocopherol and from 0.3 to 3% tween 80 and their use alone or in combination with QS21 and/or 3D-MPL), WO99/12565 (which discloses oil in water emulsion compositions comprising a metabolisable oil, a saponin and a sterol and MPL) or WO99/11241 may be used. Further oil in water emulsions such as those disclosed in WO 09/127676 and WO 09/127677 are also suitable. A particularly potent adjuvant formulation involving QS21, 3D-MPL and tocopherol in an oil in water emulsion is described in WO 95/17210. In a specific embodiment, the immunogenic composition or vaccine additionally comprises a saponin, for example QS21. The immunogenic composition or vaccine may also comprise an oil in water emulsion and tocopherol (WO 95/17210).

### Prophylactic and Therapeutic Uses

The present invention also provides methods of treating and/or preventing bacterial infections of a subject comprising administering to the subject a modified carrier protein, conjugate or bioconjugate of the invention. The modified carrier protein, conjugate or bioconjugate may be in the form of an immunogenic composition or vaccine. In a specific embodiment, the immunogenic composition or vaccine of the invention is used in the prevention of infection of a subject (e.g. human subjects) by a bacterium. Bacterial infections that can be treated and/or prevented using the modified carrier protein, conjugate or bioconjugate of the invention include those caused by Staphylococcus species, Escherichia species, Shigella species, Neisseria species, Moraxella species, Haemophilus species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Bacillus species, Clostridium species, Listeria species, or Campylobacter species, Staphylococcus aureus, N. meningitidis, H. influenzae, H. influenzae type b, Group B Streptococcus, S. typhi, M. catarrhalis LPS, S. flexneri, P. aeruginosa, E. coli or S. pneumoniae.

Also provided here are methods of inducing an immune response in a subject against a bacterium, comprising administering to the subject a modified carrier protein, or conjugate or bioconjugate of the invention (or immunogenic composition or vaccine). In one embodiment, said subject has bacterial infection at the time of administration. In another embodiment, said subject does not have a bacterial infection at the time of administration. The modified carrier protein, conjugate or bioconjugate of the invention can be used to induce an immune response against Staphylococcus species, Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Bacillus species, Clostridium species, Listeria species, or Campylobacter species. In a specific embodiment, modified Hla protein, or conjugate or bioconjugate of the invention is used to induce an immune response against Staphylococcus species (e.g. Staphylococcus aureus).

Also provided herein are methods of inducing the production of opsonophagocytic antibodies in a subject against a bacterium, comprising administering to the subject a modified carrier protein, or conjugate or bioconjugate of the invention (or immunogenic composition or vaccine). In one embodiment, said subject has bacterial infection at the time of administration. In another embodiment, said subject does not have a bacterial infection at the time of administration. The modified carrier protein, or conjugate or bioconjugate of the invention (or immunogenic composition or vaccine) provided herein can be used to induce the production of opsonophagocytic antibodies against Staphylococcus species, Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Bacillus species, Clostridium species, Listeria species, or Campylobacter species. In a specific embodiment, a modified carrier protein, or conjugate or bioconjugate of the invention (or immunogenic composition or vaccine) is used to induce the production of opsonophagocytic antibodies against Staphylococcus species (e.g. Staphylococcus aureus).

The present invention also provides methods of treating and/or preventing bacterial infections of a subject comprising administering to the subject a modified carrier protein, conjugate or bioconjugate of the invention. The modified carrier protein, conjugate or bioconjugate may be in the form of an immunogenic composition or vaccine. In a specific embodiment, the immunogenic composition or vaccine of the invention is used in the prevention of infection of a subject (e.g. human subjects) by a bacterium. Bacteria infections that can be treated and/or prevented using the modified carrier protein, conjugate or bioconjugate of the invention include those caused by Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species. In a specific embodiment, the immunogenic composition or vaccine of the invention is used to treat or prevent an infection by Streptococcus species (e.g. Streptococcus pneumoniae).

Also provided herein are methods of inducing an immune response in a subject against a bacterium, comprising administering to the subject a modified carrier protein, or conjugate or bioconjugate of the invention (or immunogenic composition or vaccine). In one embodiment, said subject has bacterial infection at the time of administration. In another embodiment, said subject does not have a bacterial infection at the time of administration. The modified carrier protein, conjugate or bioconjugate of the invention can be used to induce an immune response against Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species. In a specific embodiment, modified carrier protein, or conjugate or bioconjugate of the invention is used to induce an immune response against Streptococcus species (e.g. Streptococcus pneumoniae).

Also provided herein are methods of inducing the production of opsonophagocytic antibodies in a subject against a bacterium, comprising administering to the subject a modified carrier protein, or conjugate or bioconjugate of the invention (or immunogenic composition or vaccine). In one embodiment, said subject has bacterial infection at the time of administration. In another embodiment, said subject does not have a bacterial infection at the time of administration. The modified carrier protein, or conjugate or bioconjugate of the invention (or immunogenic composition or vaccine) provided herein can be used to induce the production of opsonophagocytic antibodies against Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species. In a specific embodiment, a modified carrier protein, or conjugate or bioconjugate of the invention (or immunogenic composition or vaccine) is used to induce the production of opsonophagocytic antibodies against Streptococcus species (e.g. Streptococcus pneumoniae).

In an embodiment, the present invention is an improved method to elicit an immune response in infants (defined as 0-2 years old in the context of the present invention) by administering a therapeutically effective amount of an immunogenic composition or vaccine of the invention (a paediatric vaccine). In an embodiment, the vaccine is a paediatric vaccine.

In an embodiment, the present invention is an improved method to elicit an immune response in the elderly population (in the context of the present invention a patient is considered elderly if they are 50 years or over in age, typically over 55 years and more generally over 60 years) by administering a therapeutically effective amount of the immunogenic composition or vaccine of the invention. In an embodiment, the vaccine is a vaccine for the elderly.

All references or patent applications cited within this patent specification are incorporated by reference herein.

In order that this invention may be better understood, the following examples are set forth. These examples are for purposes of illustration only and are not to be construed as limiting the scope of the invention in any manner.

### EXAMPLES

### Materials and Methods

### Bacterial Strains, Cloning and Growing Conditions.

The Polymerase Incomplete Primer Extension (PIPE) method (Klock H E et al., 2009, Methods Mol Biol.; 498:91-103) was applied for mutagenesis and cloning experiments to obtain the plasmids Bcp218, Bcp233, Bcp234 and Bcp235 carrying the sequence of the newly designed carriers Hla-i, Hla-s, Hla-v and Hla-a carriers (SEQ ID NOs: 36-39), in which the bioconjugation consensus sequence in position 131 of a recombinant form of Hla (SEQ ID NO: 9 in patent PCT/EP2018/085854, published as WO2019/121924; plasmid: pGVXN2872; see also Wacker, M. et al J. Infect. Dis. 2014, 209, 1551-1561) was substituted with the sequences KDSNITSAR (SEQ ID NO: 42), KDSNSTSAR (SEQ ID NO: 43), KDSNVTSAR (SEQ ID NO: 44) and KDSNATSAR (SEQ ID NO: 45), respectively. As an alternative to position 131, or in addition to position 131, these glycosite sequences have been added at the N-terminal end and/or at the C-terminal end of the Hla protein to obtain three further newly designed Hla carriers, Hla-N, 131, Hla-131,C and Hla-N,C wherein glycosite at the N-terminal end was preceded and followed by the introduction of some amino acids (GSGGG, SEQ ID NO:50) creating a spacer between the FlgI signal sequence and the starting of the protein sequence, while glycosite at the C-terminal end was only preceded by the spacer (SEQ ID Nos: 51-53). DNA plasmid encoding sequences for the designed double sites constructs were obtained from GeneArt® supplier and the DNA inserts of Hla constructs were cloned by the above-mentioned PIPE method. The transformation of chemically competent E. coli topTEN (Thermo®) by using such mutagenesis PCR reaction products allowed to obtain colonies carrying the specific plasmids constructs (Klock H E et al., 2009, Methods Mol Biol.; 498:91-103). Plasmids from selected clones were purified and sequenced to confirm and select the Hla constructs of interest.

E. coli W3110 StGVXN9268 cells were co-transformed by electroporation with plasmids encoding the S. aureus Hla-i Hla-v, Hla-s, Hla-N,131, Hla-131,C, or Hla-N,C isoform, and the C. jejuni oligosaccharyltransferase PglBN311V-K482R-D483H-A669V (pGVXN1221).

Transformed bacteria were grown overnight on selective agar plates supplemented with the two antibiotics kanamycin [50 μg/ml] and spectinomycin [80 μg/ml] for the maintenance of the plasmid encoding for Hla and PglB, respectively. Bacteria were inoculated in 50 ml Lysogeny broth (LB) containing antibiotics and shaken in Erlenmeyer flask overnight at 37° C., 180 rpm. A culture of 50 ml HTMC medium supplemented with kanamycin [50 μg/ml] and spectinomycin [80 μg/ml] was inoculated to a dilution of 0.1 optical density at 600 nm (OD_{600 nm}), incubated at 37° C. in a shaker at 180 rpm, until an average OD_{600 nm} of 0.8-1.0 and then induced overnight, by using 0.2% arabinose for the induction of Hla expression and 1 mM IPTG for the induction of PglB expression. The cultures were shaken overnight at 37° C., 180 rpm, and 20 OD_{600 nm} of bacteria were harvested after 20 hours of induction. The supernatants were discarded, and the pellets were immediately used for periplasmic extraction.

### Periplasmic Extraction

Periplasmic extraction (PPE) was performed on 20 OD_{600 nm} of bacterial pellets recovered after 20 min centrifugation (4000 rcf at 4° C.), resuspended in 600 uL of Lysis buffer (30 mM Tris HCl pH 8.5, 1 mM EDTA, 20% (wt/vol) Sucrose) and then treated for 20 min in a rotating shaker at 4° C. with 1 mg/mL final Lysozyme. After 20 min of centrifugation at 16000 rcf 4° C., supernatants were immediately collected and stored at −20° C. until their use. Total protein content was assessed by Bicinchoninic acid assay (Kit Reducing Agent Compatible, Thermo Fischer Scientific).

### Western Blot Analysis

Glycosylation status of the periplasmic Hla was analyzed by SDS-PAGE (4-12% Bis Tris in MES 1× at 150V for 1 h) and immunoblotting Hla-CP5 antisera (1:1000). A commercial secondary Goat Anti-Rabbit HRP-conjugated antibody was used in a 1:5000 dilution (DAKO Ab, Agilent, CA, USA).

### Selection of PTPs for Hla Total Protein Amount Quantification.

20 ug of recombinant Hla H35L was boiled at 95° C. for 5 minutes in 50 mM ammonium bicarbonate containing 5 mM of DTT and 0.1% (wt/vol) RapiGestSF (Waters, USA), and digested overnight at 37° C. with trypsin [1/25 (wt/wt), enzyme/substrate ratio] (Promega). Peptides mixture were analyzed by LC-MS/MS performed on an Acquity UPLC system (Waters®) coupled with a Thermo Scientific Q Exactive plus mass spectrometer equipped with a micro electron spray ESI source (Thermo). Samples were loaded using a full loop injection at a flow rate of 40 uL/min in a mobile phase A (0.1% Formic Acid FA). Peptides were than separated on a nano Acquity UPLC Peptide BEH C18 Column 75 μm×100 mm (Waters®) using a 60 min gradient 3-98% mobile phase B (98% (vol/vol) Acetonitrile, 0.1% (vol/vol) FA) at a flow rate of 40 ul/min.

The eluted peptides were run with an automated data-dependent acquisition (DDA) on top ten m/z using Xcalibur software. Peptide identification was run using Peaks X software on an E. coli K-12 database downloaded from NCBInr database, in which Hla H35L sequence was inserted. Search parameters as variable modifications were: methionine oxidation, glutamine and asparagine deamidation, trypsin cleavage (cleaves the C-term side of K or R unless next residue is P), peptide mass tolerance as 0.15 Da, peptide MS/MS tolerance as 0.15 Da, missed cleavage=2, ion charge states: +2, +3, +4).

Suitable PTPs were selected based on the following criteria: (i) peptides specific for Hla, (ii) peptides showing strong MS signal intensities either for the parental or fragment ions, (iii) peptides that do not contain methionine and tryptophan residues, which are susceptible to oxidation, or N-terminal glutamine, to avoid cyclization.

SRM-MS method set-up

Detection and chromatographic elution optimizations of the peptides were performed with 1 pmol of synthetic peptides in a mix solution of light and heavy forms in 0.1% (vol/vol of FA using a reverse phase column (ACQUITY UPLC HSS T3 Column, 100 Å, 1.8 μm, 2.1 mm×30 mm, Waters, USA) coupled to a Xevo TQ triple quadrupole mass spectrometer associated to an UPLC (Waters, USA). The elution gradient is developed with mobile phase A 0.1% (vol/vol) FA in water and B 0.1% (vol/vol) FA in acetonitrile. The synthetic peptides were used to optimize collision energy (CE) values starting from the theoretical value, computed in silico by using PinPoint software calculated using the formula CE=0.034×(parent m/z)+1.314 (MacLean B. et al., Anal. Chem. 2010; 82:10116-10124), and to validate the transitions in 50 μg matrix. The optimization of the chromatographic separation was performed in an SRM acquisition mode by using the optimized CE and the selected transitions, both in neat and in matrix background (see Table 3).

### Sample Preparation for LC-SRM Analysis of Bacterial Periplasmic Extract.

50 ug of PPE fractions were digested by using an in-stage-tips (iST) sample preparation kit supplied by PreOmics (Martinsried, Germany). It is a 3-step protocol performed on a cartridge: 1) lysis, denaturation, reduction and alkylation; 2) proteolytic digestion by LysC and Trypsin; 3) peptide desalting operated as recommended by the provider. Recovered peptides were dried under vacuum at 45° C. and resuspended in 0.1% (vol/vol) FA to a final concentration of 1 ug/uL and stored at −20° C. until the MS analysis.

### SRM-MS Analysis.

SRM was performed by injecting 10 ug of a periplasmic fraction digested with IsT sample preparation kit in column per run, and each sample was analyzed in triplicate. The following parameters were used: Q1 isolation window 1.0 m/z, Q3 Isolation window 0.7 m/z, 0.03 s of switching time (dwell time) from MS to MS/MS and collision cell exit and entrance potential set at 30 V. A spray voltage of 1,700 V was used with a heated ion transfer setting of 270° C. for desolvation. Data were acquired using MassLynx software (version 2.1.0; Waters). The dwell time was set to 30 ms and the scan width to 0.02 m/z. The peak area quantification was determined with TargetLynx software (version 1.0.0.1; Waters) after confirming the coelution of all transitions for each peptide and following the best practices reported in Carr et al., Mol Cell Proteomics 13(3):907-917, 2014.

### PTP Dose-Range Linearity Responses and Hla Quantification.

The dose-range linearity response of the selected PTPs was assessed in a periplasmic bacterial sample prepared from E. coli glycocompetent cells (stGVXN9268 transformed with PglB plasmid) used as reference background to consider the matrix effect.labeled PTPs (final concentration 0.1 pmol/μL) and non-labeled PTPs (final concentration from 1.6 pmol/μl to 0.0125 pmol/μl) were spiked in 50 μg of periplasmic fraction prior to digestion with IsT sample preparation kit,

For each PTP, concentrations were plotted as ratio of peak area light (variable)/peak area heavy (constant) and the fitted curve was used to obtain the concentration of selected endogen PTP. According to the International Conference on Harmonization (ICH) Guidelines (http://www.ich.org/products/guidelines/quality/article/quality-guidelines.html), the lower limit of quantification (LLOQ) for each peptide was set as the lowest concentration point on the fitted curve that can be quantitively detected and defined as 10 σ/S, where σ=the standard deviation of the response and S=the slope of the calibration curve.

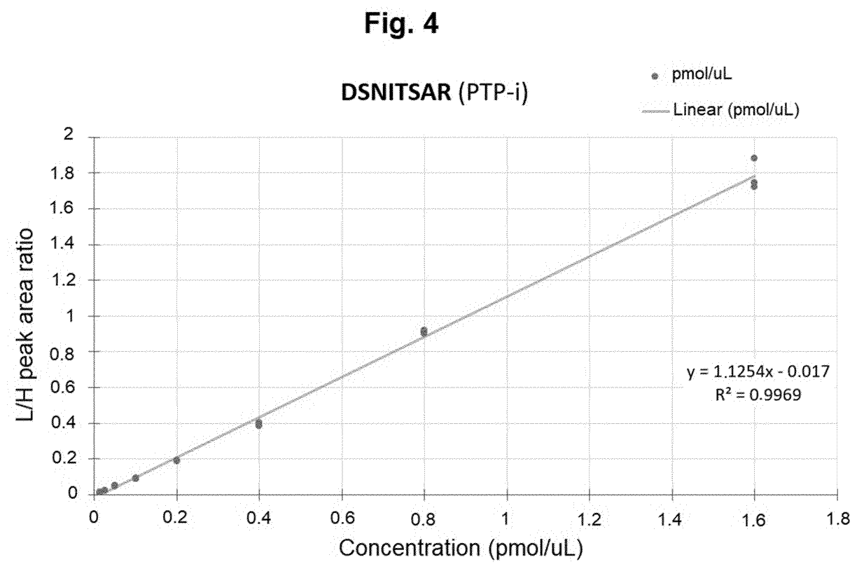

The Hla concentrations were reported in picograms per microgram of total periplasmic protein extract considering the molecular mass average of the Hla-i (34093.07 Da), Hla-s (34066.99 Da), Hla-v (34079.05 Da), Hla-N,131 (36962.06 Da), Hla-131,C (36518.60 Da) and Hla-N,C (37390.51 Da) isoforms. The quantifications were obtained by the interpolation of each peptide-response value in the related dose-response linearity curve (FIG. 4).

### Results

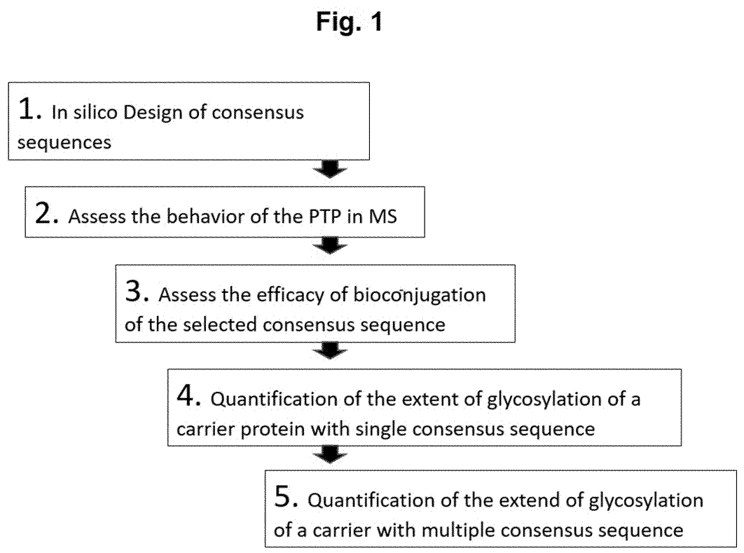

The workflow undertaken to design new carrier proteins for bioconjugation is reported in FIG. 1.

The first step was the in-silico design of consensus sequences, predicted to be substrates of the PglB enzyme (Kowarik et al, 2006, EMBO J. 25:1957-1966) and able to generate tryptic peptides (referred as proteotypic peptides PTPs) suitable for the quantification of the extent of glycosylation by MS (Zhu et al, 2014, J Am Soc Mass Spectrom. 25:1012-7).

The designed PTPs were chemically synthetized in natural or heavy-labelled forms by incorporating 13C-15N in the arginine residue and investigated for their behavior in MS/MS analysis using a triple quadrupole instrument.

Once PTPs suitable for quantification by MS were identified, the corresponding sequences were introduced in a carrier protein, and the efficiency of PglB enzyme to recognize and glycosylate the new carrier was evaluated.

The site occupancy for each consensus sequence was then determined from the absolute quantification of the non-glycosylated form of the glycopeptide, by using isotopically labeled internal standards and a SRM approach. Two sets of heavy isotope labeled peptide standards were spiked into the sample before proteolysis, and LC-SRM MS. One set of peptide standard was employed to determine the total carrier concentration, while the other standard set monitored the non-glycosylated part of the carrier. In this way, the abundance of the glycosylated portion of the protein was calculated by subtracting the non-glycosylated protein abundance from the overall protein concentration, and the site occupancy was then determined. The approach has been demonstrated to be successful for the quantification of naturally glycosylated eukaryotic protein (Zhu et al, 2014, J Am Soc Mass Spectrom. 25:1012-7).

As a proof of concept, newly designed consensus sequences were introduced into a recombinant form of S. aureus Hla, a substrate used as a carrier protein for the bioconjugation of S. aureus CP5 (see PCT/EP2018/085854, published as WO2019/121924). The carrier has been reported to be efficiently glycosylated by the insertion, in the position 131 of the consensus sequence, (−3)KDQNRTK(+3), where the Asn residue (in position 0) is the glycan acceptor. Unfortunately, it was found that this antigen design was not adapted for the quantification of glycosylation extent since digestion of the carrier by trypsin generated an unmodified peptide (−2)DQNR(+1) that was too short and hydrophilic to be monitored by LC-MS/MS. Different resin and gradients were tested without any success.

### In-Silico Design of Consensus Sequences

The consensus sequence substrate of C. jejuni PglB has been well characterized (Kowarik et al, 2006, EMBO J. 25:1957-1966). The sequence is characterized by the presence of negatively charged side chain amino acid residues in the −2 position (asp or glu), and a ser or thr in position +2 of the asn acceptor of the saccharide. Moreover, an efficient bioconjugation also requires that the consensus sites are in accessible and flexible loops of the carrier protein (Silverman et al., 2016, J. Biol. Chem. 291, 22001-22010).

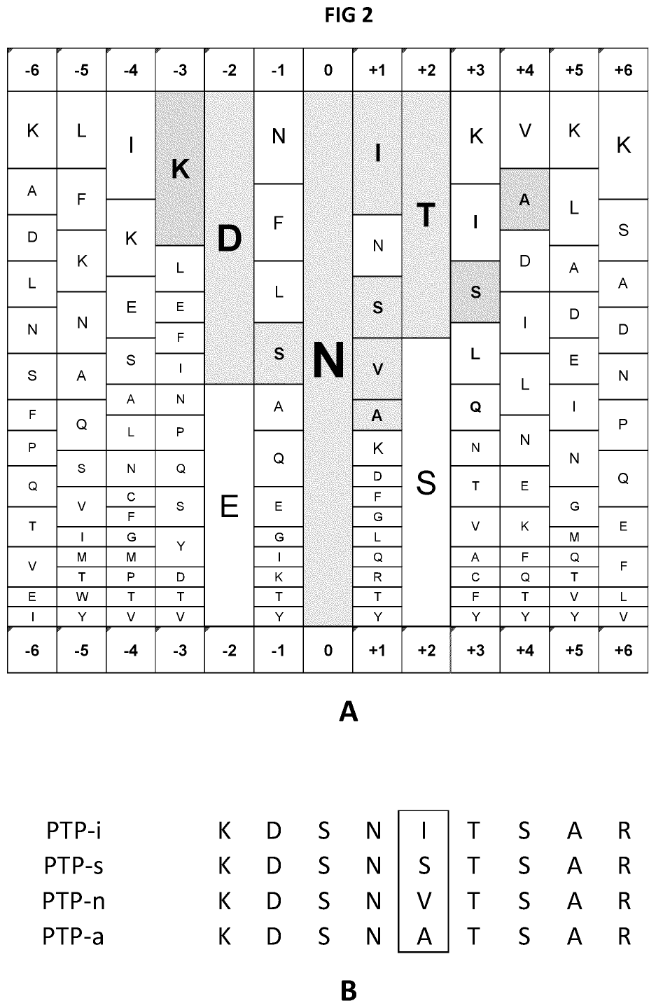

A statistical analysis of the occurrence of amino acids in the region from −6 to +6 of the glycosylated asn residue found in 32 native C. jejuni glycoproteins is reported in Kowarik et al. EMBO J. 2006; 25(9): 1957-66, as shown in FIG. 2A. The amino acid residues in position −3, −1, +1, +3 and +4, respectively, represented in bold in grey boxes, were selected for the design of the four consensus sequences (FIG. 2B). These residues were selected as frequently found and responding to the set-up criteria. The amino acid arg in position +5 (not reported in the statistical analysis), and the amino acid lys in position −3 are the substrates of trypsin, required for the generation of the PTPs. The PTPs differed from each other only from the amino acid residue in position +1.

With these minimal requirements in mind, four consensus sequences predicted to be substrates of the PglB and able to generate tryptic peptides were designed (FIG. 2B). In detail, the following criteria were taken in to account: (i) to circumvent possible interference with carrier structure, the inserted consensus sequences did not exceed nine amino acid residues and the insertion of hydrophobic and aromatic amino acid residues was limited, (ii) to avoid underestimated quantification, amino acid residues prone to post-translational modifications such as oxidation (met, cys, trp) and deamination (asn and gln) were limited, and the consensus sequences were designed to be substrate of trypsin, selected for its high specificity, efficacy and ability to generate C-terminal positively charged peptides, (iii) preferential amino acid residues surrounding the asn, acceptor of the saccharide, evidenced from the comparison of a data set containing 32 active C. jejuni N-glycosylation sites were taken in consideration (4), and (iv) the newly designed consensus sequences were unique for the newly designed carrier isoforms.

The four designed consensus sequences were (−3)KDSNXTSAR(+5) in which X is an Ile, Ser, Val or Ala amino acid residue. After LysC/trypsin digestion, PTPs (−2)DSNXTSAR(+5), named PTP-i (SEQ ID NO:42), PTP-s (SEQ ID NO:43), PTP-v (SEQ ID NO:44) and PTP-a (SEQ ID NO:45) according to the amino acid residue present in position +1 are generated (FIG. 2B).

### SRM Assay for Quantification of Extent of Glycosylation

The behaviors of chemically synthesized PTP-i-s-v-a were evaluated in SRM assays. The four PTPs were separated on a reverse phase C18 on-line with a triple-quadrupole mass spectrometer, with well distinct retention time ranging from 1.65 to 5.74 min (the minimal difference of 0.5 min was observed between peptide PTP-a and PTP-s) and with strong MS signals.

For each PTPs, four transitions (precursor/product pairs b4, y5, and y6 containing the selective amino acid residue specific for each glycosylation site, and y4, common to all peptides) were computed by Pin Point software and first optimized by in-neat injection (Table 1).

The transitions were then validated in an E. coli periplasmic fraction digested with LysC/Trypsin to evaluate the effect of the matrix (Lange et al, 2008, Mol. Syst. Biol. 4:222). While the matrix had minor effects on the PTP-s, PTP-v and PTP-i performances (Table 1), it had deleterious effect on PTP-a, for which neither retention time and transitions were stable over repetitive analysis. For this reason, the bioconjugation consensus sequence (−5)KDSNATSAR(+3) was not further investigated.

For PTP-s, PTP-v and PTP-i, collision energies were optimized and a dose-response linearity curve was established adding to 50 μg E. coli periplasmic fraction a fixed amount of heavy forms of PTPs (0.1 pmol/pg) and scalar concentration of light PTPs (ranging from 0.0125 to 1.6 pmol/pg), before the trypsin digestion. According to the International Conference on Harmonization (ICH) Guidelines (http://www.ich.org/products/guidelines/quality/article/quality-guidelines.html), the LLOQ for each peptide was set as the lowest concentration point on the fitted curve that can be quantitively detected and defined as 10 σ/S, where σ=the standard deviation of the response and S=the slope of the calibration curve. Also the LOD or Limit of Detection (LOD=3.3 σ/S) was defined based on ICH Guidelines (FIG. 4).

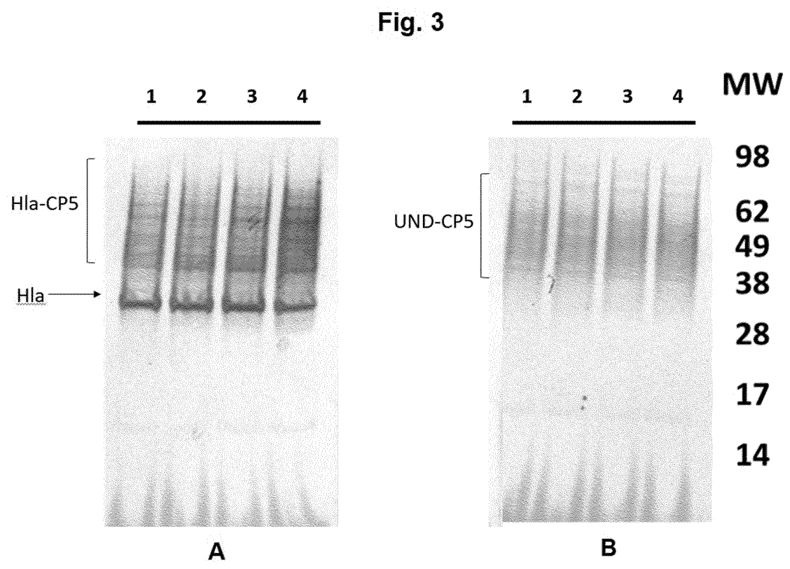

The selected consensus sequences are substrates of PglB The three newly designed consensus sequences PTP-i (SEQ ID NO: 42), PTP-s (SEQ ID NO: 43), and PTP-v (SEQ ID NO: 44) were inserted in position 131 of an optimized Hla antigen (see PCT/EP2018/085854, published as WO2019/121924) to generate the bioconjugates Hla-i-CP5, Hla-v-CP5 and Hla-s-CP5, which produce the PTP-i, PTP-v, PTP-s peptides, respectively, once digested with LysC/trypsin. Periplasms of glycocompetent E. coli strains bearing the machinery required for the bioconjugation were isolated and the conjugation of CP5 to Hla was assessed by Western-blot analysis using a murine serum that recognizes Hla-CP5 bioconjugate (FIG. 3). The carriers are characterized by a partial glycosylation pattern that was comparable among the three different constructs, although the extent of glycosylation could not be quantified from the Western blot.

### Quantification of Extent of Hla-CP5 Glycosylation

The extent of glycosylation was assessed by SRM. In detail, site occupancies were determined by the following equation (Zhu et al 2015, J Am Soc Mass Spectrom. 25:1012-7):

$$Site Occupancy ( \% ) = \frac{( Total - unmodified ) carrier concentration}{Total carrier concentration} \times 100$$

where the unmodified carrier concentrations were determined by the quantification of endogenous PTP-i, PTP-v, PTP-s and the total carrier concentrations were quantified by peptides specific for Hla.

To identify suitable Hla-specific peptides, recombinant Hla was digested with trypsin and the generated peptides were analyzed by LC-MS/MS. Two peptides, 42T-50K (TGDLVTYK, SEQ ID NO: 48) and 225A-234K (AADNFLDPNK, SEQ ID NO: 49), named PTP₋₂ and PTP₋₁ respectively were selected. All the information regarding the two peptides (transitions, optimized CE, retention time and LLOQ in the matrix) are reported in Table 3.

Moreover, the SRM assay requires effective protease digestions of the carrier to ensure consistency in the quantification of each selected PTP. The efficiency of the digestion was checked by SDS/PAGE and by assessing by LC-MS/MS that the number of missed cleavages was inferior to 2% of the total identified peptides (Biagini M et al, 2016, Proc Natl Acad Sci USA. 113:2714-9)

The interchangeability of the PTP-i-v-s and PTP₋₁₋₂ was demonstrated by spiking recombinant Hla, and known amount of each isotopically labeled PTPs in an E. coli periplasmic fraction before trypsin digestion.

Periplasmic fractions were isolated from E. coli glycocompetent strains expressing the bioconjugates of different newly designed Hla carriers with CP5 (Hla-i-CP5, Hla-v-CP5, and Hla-s-CP5), and a known amount of each isotopically labeled PTP was added before the LysC/trypsin digestion and analysis by LC-SRM. The experiments were performed from three independent digestions run in triplicate. The quantification of the PTPs were very reproducible between the runs with almost all coefficient of variation (CV) inferior to 10% while the CV associated to the reproducibility of the digestion and the quantification of the extend of bio-conjugation were inferior to 14% (Table 1). These values are in line with CVs reported in literature and with the error intended of the analysis (Hüttenhain et al 2009, Curr. Opin. Chem. Biol. 13 518-525). The concentrations of total and un-glycosylated amount of the carriers allowed to determine that Hla-i-CP5, Hla-v-CP5 and Hla-s-CP5 represented 41.47%, 45.14% and 42.73% of the total amount of the carrier expressed (Table 4). The similar extent of glycosylation was in agreement with the pattern observed in Western blot analysis (FIG. 3). The introduction of the variable amino acid residue (ser, val, or ile) in the consensus sequence did not significantly affected the efficacy of PglB enzyme to bioconjugate Hla carrier. The identification of three different consensus sequences allowed the design of Hla carriers bearing multiple quantifiable conjugation sites, as described below.

Design of Hla Construct Bearing Two Glycosylation Sites

Hla carriers with multiple consensus sequences substrate, suitable for quantification of glycosylation extent, were designed by inserting PTP-i and PTP-v in alternative combinations at the N-Terminal, C-Terminal, and/or in position 131 of the Hla protein. In accordance with their respective positions of the consensus sequence insertions, they are designated here as Hla-N,131 (SEQ ID NO:50), Hla-131,C (SEQ ID NO:51), and Hla-N,C (SEQ ID NO:52).

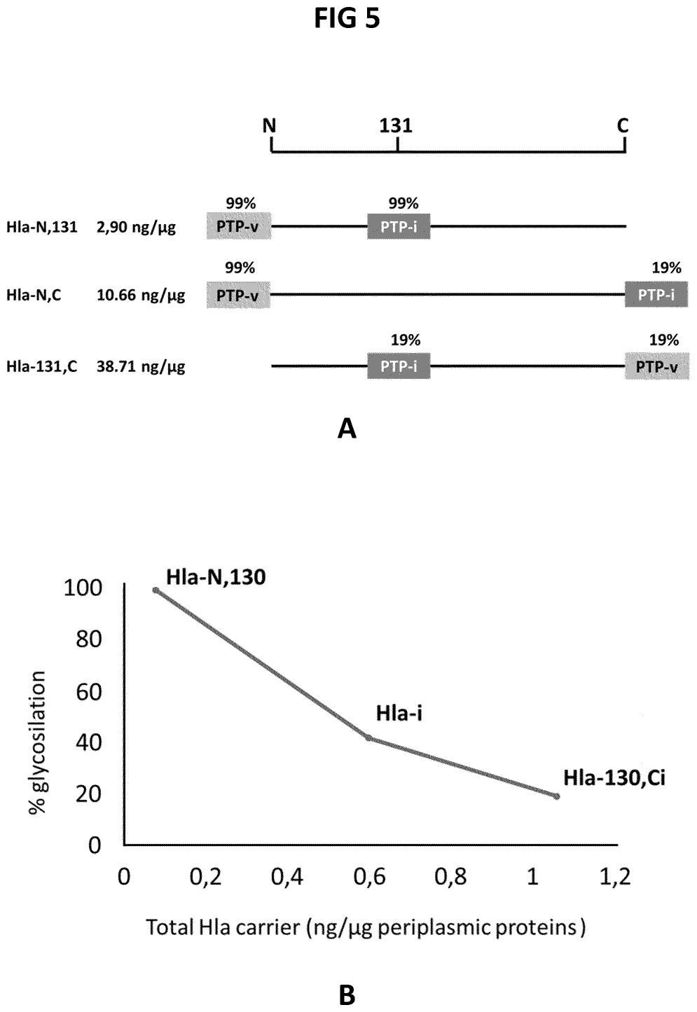

The quantification of the three carriers and the calculated extent of glycosylation are reported in the following Table 5 and summarized in the FIG. 5A.

A low amount of carrier Hla-N,131 (2.90 ng/pg total periplasmic proteins) was quantified in the periplasmic extract and both peptides containing the glyco-sequence (PTP-i and PTP-v) resulted not quantifiable with values detected under the LLOQ of the analysis. For this reason, both consensus sequences were considered fully conjugated. For the Hla-N,C isoform, the quantification resulted to be 10.66 ng/pg of periplasmic extract. The peptide carrying the glyco-sequence on the N-terminal domain (PTP-v) resulted fully glycosylated, while only ˜19% of glycosylation extent was achieved for the glyco-sequence in C-terminal position (PTP-i). The Hla-131,C isoform resulted to be the most expressed isoform, with 38.71 ng/pg of the total periplasmic proteins with a glycosylation extent of around 19% in position 131 (PTP-i) as well as at the C-terminal end (PTP-v).

These data showed that the measure of the extent of glycosylation by the method of this invention can be assessed on individual sites of bioconjugation also when several sites are simultaneously inserted in the carrier protein. Moreover, the glycosite located at N-terminal domain of the carrier protein resulted fully glycosylated in all the isoforms analyzed. Instead, when the glycosite was inserted in position 131, which is a Hla flexible and solvent-exposed loop, the extent of glycosylation was inversely proportional to the carrier protein amount independently from the presence or not of a second site of glycosylation (FIG. 5B). Finally, when a glycosite was inserted in the more structured C-terminal region, the extent of glycosylation was similar for the two carrier proteins tested Hla-131,C and Hla-N,C indicating that PglB was also able to some extent to perform N-glycosylation on folded protein portion (Fisher A. C. et al., 2011, Applied and Environmental Microbiology, 77(3) 871-881).

TABLE 1
List of optimized SRM transitions for the selected PTPs. For each PTP, PTPs name, peptide sequence, molecular
mass, and the optimized transition and chromatographic condition stablished in SRM analysis are reported.
For each selected fragment, the reproducibility of detection was assessed monitoring the TIC signal.

| | | | | | | | | 1 pmol | | | 1 pmol in matrix | | |
|---|---|---|---|---|---|---|---|---|---|---|---|---|---|
| | | Molecular | | Q1 (m/z) | | Q3 (m/z) | | loaded on column | | | loaded on column | | |
| Peptide | | mass | Charge | precursor | | fragment | Fragment | | | Channel | | | Channel |
| name | Sequence | (Da) | state | ion | CE | ions | ions | RT | TIC | intensity | RT | TIC | intensity |
| PTP-i | DSNITSAR | 862.89 | 2 | 432.21 | 16 | 434.21 | y4 | 5.74 | 4.4E+04 | 30700 | 4.97 | 4.49E+04 | 33800 |
| | (SEQ ID NO: 42) | | | | 14 | 547.15 | y5 | | | 8030 | | | 5140 |
| | | | | | 14 | 661.47 | y6 | | | 7480 | | | 5700 |
| | | | | | 12 | 430.20 | b4 | | | 3980 | | | 2610 |
| PTP-s | DSNSTSAR | 836.81 | 2 | 419.19 | 16 | 434.27 | y4 | 1.65 | 3.32E+04 | 12000 | 1.66 | 3.3E+03 | 1070 |
| | (SEQ ID NO: 43) | | | | 14 | 521.39 | y5 | | | 15100 | | | 1640 |
| | | | | | 14 | 634.96 | y6 | | | 8010 | | | 1180 |
| | | | | | 16 | 404.14 | b4 | | | 2480 | | | 145 |
| PTP-v | DSNVTSAR | 848.97 | 2 | 425.21 | 16 | 434.23 | y4 | 2.02 | 8.00E+04 | 46000 | 2.05 | 3.47E+04 | 18600 |
| | (SEQ ID NO: 44) | | | | 18 | 533.30 | y5 | | | 12400 | | | 5400 |
| | | | | | 18 | 647.34 | y6 | | | 9670 | | | 6070 |
| | | | | | 12 | 416.38 | b4 | | | 12000 | | | 6690 |
| PTP-a | DSNATSAR | 820.81 | 2 | 411.19 | 15 | 434.24 | y4 | 1.70 | 3.68E+04 | 11400 | — | — | — |
| | (SEQ ID NO: 45) | | | | 17 | 505.27 | y5 | | | 10300 | | | — |
| | | | | | 17 | 619.32 | y6 | | | 12400 | | | — |
| | | | | | 13 | 388.27 | b4 | | | 4710 | | | — |

TABLE 2
Optimised chromatographic gradient

| Time | mL/min | % A | % B |
|---|---|---|---|
| 0.00 | 0.080 | 97.0 | 3.0 |
| 1.00 | 0.080 | 97.0 | 3.0 |
| 3.00 | 0.080 | 95.0 | 5.0 |
| 10.00 | 0.080 | 65.0 | 35.0 |
| 13.00 | 0.100 | 10.0 | 90.0 |
| 15.00 | 0.100 | 10.0 | 90.0 |
| 15.01 | 0.100 | 10.0 | 90.0 |
| 17.00 | 0.100 | 93.0 | 7.0 |
| 17.01 | 0.100 | 93.0 | 7.0 |

TABLE 3
Information regarding the two peptides PTP₋₂ and PTP₋₁
that are 42T-50K (TGDLVTYK, SEQ ID NO: 48) and
225A-234K (AADNFLDPNK, SEQ ID NO: 49):
transitions, optimized CE, retention time
and LLOQ in the matrix

| | | Q1 | Q3 | | | | | LLOQ in |
|---|---|---|---|---|---|---|---|---|
| Peptide | | Precursor | Fragment | Fragment | CE | Charge | RT | matrix |
| name | Sequence | ion (m/z) | ions (m/z) | ions | optimized | state | (min) | (pmol/ug) |
| PTP-2 | TGDLVTDK | 506.2532 | 526.25 | y4 | 18 | 2 | 7.42 | 0.053 |
| | (SEQ ID | | 625.32 | y5 | 18 | 2 | | |
| | NO: 48) | | 853.43 | y7 | 18 | 2 | | |
| PTP-2* | TGDLV | 510.2603 | 534.26 | y4 | 18 | 2 | 7.42 | 0.0053 |
| | TDK | | 633.33 | y5 | 18 | 2 | | |
| | (SEQ ID | | 861.44 | y7 | 18 | 2 | | |
| | NO: 48) | | | | | | | |
| PTP-1 | AADNF | 552.7696 | 473.23 | y4 | 18 | 2 | 8.30 | 0.050 |
| | LDPNK | | 586.32 | y5 | 18 | 2 | | |
| | (SEQ ID | | 962.46 | y8 | 18 | 2 | | |
| | NO: 49) | | 733.39 | y6 | 18 | 2 | | |
| | | | 358.21 | y3 | 18 | 2 | | |
| PTP-1* | AADNF | 556.7767 | 481.25 | y4 | 18 | 2 | 8.30 | 0.050 |
| | LDPNK | | 594.33 | y5 | 18 | 2 | | |
| | | | 970.47 | y8 | 18 | 2 | | |
| | (SEQ ID | | 741.40 | y6 | 18 | 2 | | |
| | NO: 49) | | 366.22 | y3 | 18 | 2 | | |

TABLE 4
Quantification of the PTPs for the definition of % site occupancy. For each Hla isoform (Hla-i-v-s) 3 proteolytic digestions were
performed, quantifying both the total HLA and the unglycosylated form using the 2 sets of PTPs PTP-1-2 and PTP-i-v-s
respectively. For each digestion the PTP amount are reported in pmol/μg of total periplasmic protein extract and used to calculate,
from each digestion, the extent of bio-conjugation. The CV between runs and digestion are reported. The obtained % site
occupancies for each isoform were reported as an average of the % sites occupancies deduced from each digestion,
moreover, moreover, the total amount of Hla isoforms are reported as ng/μg of total periplasmic protein extract using
each Hla construct, the average MW as reported in the table (grey boxes) indicated below.

| | Hla-i (average MW: 34093.07 Da) | | | | | | | | |
|---|---|---|---|---|---|---|---|---|---|
| | Digestion 1 | | | Digestion 2 | | | Digestion 3 | | |
| | PTP-1 | PTP-2 | PTI-i | PTP-1 | PTP-2 | PTI-i | PTP-1 | PTP-2 | PTI-i |
| run1 | 0.732 | 0.669 | 0.434 | 0.604 | 0.516 | 0.286 | 0.885 | 0.818 | 0.526 |
| run2 | 0.719 | 0.665 | 0.418 | 0.603 | 0.512 | 0.297 | 0.879 | 0.812 | 0.545 |
| run3 | 0.729 | 0.670 | 0.410 | 0.604 | 0.512 | 0.295 | 0.885 | 0.828 | 0.534 |
| CV(%) run | 0.94 | 0.40 | 2.90 | 0.10 | 0.45 | 2.00 | 0.39 | 1.00 | 1.78 |
| Average PTP-1 &2 or PTP-i | 0.697 | | 0.421 | 0.559 | | 0.293 | 0.851 | | 0.535 |
| % SITE OCCUPANCY | | | 39.67 | | | 47.60 | | | 37.14 |
| | Amount carrier: 23.94 ng/μg periplasmic proteins | | | | | | | | |
| | SITE OCCUPANCY: 41.47—CV (%): 13.16 | | | | | | | | |
| | Hla-v (average MW: 34079.05 Da) | | | | | | | | |
| | Digestion 1 | | | Digestion 2 | | | Digestion 3 | | |
| | PTP-1 | PTP-2 | PTI-v | PTP-1 | PTP-2 | PTI-v | PTP-1 | PTP-2 | PTI-v |
| run1 | 0.561 | 0.522 | 0.288 | 0.475 | 0.434 | 0.231 | 0.845 | 0.805 | 0.497 |
| run2 | 0.561 | 0.519 | 0.293 | 0.477 | 0.435 | 0.243 | 0.85 | 0.794 | 0.45 |
| run3 | 0.54 | 0.523 | 0.291 | 0.467 | 0.434 | 0.249 | 0.865 | 0.796 | 0.475 |
| CV(%) run | 2.19 | 0.40 | 0.87 | 1.12 | 0.13 | 3.80 | 1.22 | 0.73 | 4.96 |
| Average PTP-1 &2 or PTP-v | 0.538 | | 0.291 | 0.454 | | 0.241 | 0.826 | | 0.474 |
| % SITE OCCUPANCY | | | 45.94 | | | 46.88 | | | 42.60 |
| | Amount carrier: 20.64 ng/μg periplasmic proteins | | | | | | | | |
| | % SITE OCCUPANCY: 45.14—CV (%): 4.98 | | | | | | | | |
| | Hla-s (average MW: 34066.99 Da) | | | | | | | | |
| | Digestion 1 | | | Digestion 2 | | | Digestion 3 | | |
| | PTP-1 | PTP-2 | PTI-s | PTP-1 | PTP-2 | PTI-s | PTP-1 | PTP-2 | PTI-s |
| run1 | 0.692 | 0.623 | 0.336 | 0.546 | 0.502 | 0.286 | 0.787 | 0.727 | 0.451 |
| run2 | 0.692 | 0.627 | 0.372 | 0.578 | 0.499 | 0.294 | 0.789 | 0.741 | 0.429 |
| run3 | 0.687 | 0.628 | 0.387 | 0.545 | 0.498 | 0.321 | 0.777 | 0.733 | 0.474 |
| CV(%) run | 0.42 | 0.42 | 7.18 | 3.37 | 0.42 | 6.11 | 0.82 | 0.96 | 4.99 |
| Average PTP-1 &2 or PTP-s | 0.658 | | 0.365 | 0.528 | | 0.300 | 0.759 | | 0.451 |
| % SITE OCCUPANCY | | | 44.54 | | | 43.12 | | | 40.54 |

Amount carrier: 22.09 ng/μg periplasmic proteins
SITE OCCUPANCY: 42.73 %—CV (%): 4.75

TABLE 5
Quantification of the PTPs for the definition of % site occupancy. For each isoform Hla-N, 131, Hla-N, C, and
Hla-131, C three proteolytic digestions Dig1, Dig2 and Dig3 were performed, quantifying both the total HLA
and the unglycosylated form using the 2 sets of PTPs PTP-1-2 and PTP-i-v respectively. For each digestion the
PTP amount are reported in pmol/μg of total periplasmic protein extract and used to calculate, from each
digestion, the extent of bio-conjugation. The CV between runs and digestion are reported. The obtained
% site occupancies for each isoform wre reported as an average of the % sites occupancies deduced from
each digestion, moreover, the total amount of Hla isoforms are reported as ng/μg of total periplasmic
protein extract using each Hla construct, the average MW as reported in the table (grey boxes).

| | Hla-N, 131 (average MW: 336962.06 Da) | | | | | | | | | | | |
|---|---|---|---|---|---|---|---|---|---|---|---|---|
| | Digestion 1 | | | | Digestion 2 | | | | Digestion 3 | | | |
| | PTP-1 | PTP-2 | PTP-i | PTP-v | PTP-1 | PTP-2 | PTP-i | PTP-v | PTP-1 | PTP-2 | PTP-i | PTP-v |
| run1 | 0.094 | 0.086 | <LLOQ | <LLOQ | 0.075 | 0.065 | <LLOQ | <LLOQ | 0.079 | 0.072 | <LLOQ | <LLOQ |
| run2 | 0.095 | 0.086 | | | 0.074 | 0.066 | | | 0.079 | 0.071 | | |
| run3 | 0.095 | 0.087 | | | 0.073 | 0.065 | | | 0.08 | 0.071 | | |
| CV(%) run | 0.61 | 0.67 | — | — | 1.35 | 0.88 | — | — | 0.73 | 0.81 | — | — |
| Average PTP-1 | 0.091 | | — | — | 0.070 | | — | — | 0.075 | | — | — |
| &2 or PTP-i | | | | | | | | | | | | |
| or PTP-v | | | | | | | | | | | | |
| % SITE | | | >99% | >99% | | | >99% | >99% | | | >99% | >99% |
| OCCUPANCY | | | | | | | | | | | | |
| | Amount carrier: 2.90 ng/μg periplasmic proteins | | | | | | | | | | | |
| | SITE OCCUPANCY PTP-i: >99% | | | | | | | | | | | |
| | SITE OCCUPANCY PTP-v: >99% | | | | | | | | | | | |
| | Hla-131, C (average MW: 36518.60 Da) | | | | | | | | | | | |
| | Digestion 1 | | | | Digestion 2 | | | | Digestion 3 | | | |
| | PTP-1 | PTP-2 | PTP-i | PTP-v | PTP-1 | PTP-2 | PTP-i | PTP-v | PTP-1 | PTP-2 | PTP-i | PTP-v |
| run1 | 1.119 | 1.079 | 0.888 | 0.955 | 0.956 | 0.922 | 0.748 | 0.759 | 1.152 | 1.123 | 0.955 | 0.94 |
| run2 | 1.125 | 1.068 | 0.885 | 0.949 | 0.938 | 0.987 | 0.742 | 0.771 | 1.167 | 1.114 | 0.962 | 0.979 |
| run3 | 1.129 | 1.075 | 0.904 | 0.952 | 0.947 | 0.896 | 0.737 | 0.732 | 1.163 | 1.121 | 0.93 | 0.987 |
| CV(%) run | 0.45 | 0.52 | 1.14 | 0.32 | 0.95 | 5.01 | 0.74 | 2.65 | 0.67 | 0.42 | 1.77 | 2.60 |
| Average PTP-1 | 1.099 | | 0.892 | 0.952 | 0.941 | | 0.742 | 0.754 | 1.140 | | 0.949 | 0.969 |
| &2 or PTP-i | | | | | | | | | | | | |
| or PTP-v | | | | | | | | | | | | |
| % SITE | | | 18.817 | 16.492 | | | 21.112 | 25.191 | | | 16.754 | 18.054 |
| OCCUPANCY | | | | | | | | | | | | |
| | Amount carrier: 38.71 ng/μg periplasmic proteins | | | | | | | | | | | |
| | SITE OCCUPANCY PTP-i: 18.98%—CV (%): 11.54 | | | | | | | | | | | |
| | SITE OCCUPANCY PTP-v: 19.12%—CV (%): 23.29 | | | | | | | | | | | |
| | fila-N, C (average MW: 37390.51 Da) | | | | | | | | | | | |
| | Digestion 1 | | | | Digestion 2 | | | | Digestion 3 | | | |
| | PTP-1 | PTP-2 | PTP-i | PTP-v | PTP-1 | PTP-2 | PTP-i | PTP-v | PTP-1 | PTP-2 | PTP-i | PTP-v |
| run1 | 0.26 | 0.24 | 0.205 | <LLOQ | 0.325 | 0.299 | 0.242 | <LLOQ | 0.308 | 0.284 | 0.238 | |
| run2 | 0.257 | 0.241 | 0.2 | | 0.325 | 0.294 | 0.246 | | 0.315 | 0.281 | 0.241 | |
| run3 | 0.258 | 0.24 | 0.201 | | 0.319 | 0.296 | 0.252 | | 0.312 | 0.279 | 0.251 | |
| CV(%) run | 0.59 | 0.24 | 1.31 | — | 1.07 | 0.85 | 2.04 | — | 1.13 | 0.89 | 2.80 | — |
| Average PTP-1 | 0.249 | | 0.202 | — | 0.310 | | 0.247 | — | 0.297 | | 0.243 | |
| &2 or PTP-i | | | | | | | | | | | | |
| or PTP-v | | | | | | | | | | | | |
| % SITE | | | 18.98 | >99% | | | 20.34 | >99% | | | 17.93 | >99% |
| OCCUPANCY | | | | | | | | | | | | |

Amount of carrier: 10.66 ng/μg periplasmic proteins
SITE OCCUPANCY PTP-i: 18.98%—CV (%): 6.34
SITE OCCUPANCY PTP-v: >99%

Aspects of the invention are summarized in following numbered paragraphs:
1. A modified carrier protein, modified in that it comprises one or more consensus sequence(s) comprising or consisting of the following amino acid sequence:
   K/R-Z₀₋₉-D/E-X-N-Y-S/T-Z₀₋₉-K/R
wherein X and Y are independently any amino acid except proline, and Z represents any amino acid; wherein optionally X and Y are independently any amino acid except proline, lysine or arginine, Z represents any amino acid except lysine or arginine, and preferably Z represents any amino acid except cysteine, methionine, asparagine, glutamine, lysine or arginine (eg SEQ ID NO: 47)
2. A modified carrier protein according to paragraph 1, wherein said consensus sequence is the amino acid sequence K/R-D/E-X₁-N-X₂-S/T-Z₁-Z₂-K/R (SEQ ID NO 19), wherein X₁ and X₂ are independently any amino acid apart from proline, and wherein X₁ and X₂ and Z₁ and Z₂. are preferably not lysine or arginine, wherein optionally, wherein X₁ and X₂ and Z₁ and Z₂. are not cysteine, asparagine, glutamine, methionine or arginine.
3. A modified carrier protein according to paragraph 1 or paragraph 2, wherein said consensus sequence comprises or consists of the amino acid sequence of SEQ ID NO: 20, optionally any one of SEQ ID Nos: 42-45, optionally SEQ ID Nos 42-44.
4. A modified carrier protein according to anyone of paragraphs 1-3, wherein said consensus sequence (i) has been substituted for one or more amino acids of the carrier protein sequence, or (ii) has been inserted into the carrier protein sequence.
5. A modified carrier protein according to any one of paragraphs 1-4, comprising more than one said consensus sequence, optionally at least 2, 3, 4 or 5 consensus sequences.
6. A modified carrier protein according to paragraph 5, wherein all of said consensus sequences have a different amino acid sequence.
7. A modified carrier protein according to any one of paragraphs 1-6, wherein the carrier protein is CRM197, TT from Clostridium tetani, EPA from P. aeruginosa, Hcp1 from P. aeruginosa, Hla from S. aureus, ClfA from S. aureus, MBP from E. coli, PspA from E. coli, or MtrE from N. gonorrhoeae.
8. A modified carrier protein according to paragraph 7, wherein the carrier protein comprises or consists of an amino acid sequence of any one of SEQ ID Nos: 1 to 16 or an amino acid sequence at least 80%, 85%, 90%, 92%, 95%, 96%, 97%, 98% or 99% identical to any one of SEQ ID NOs. 1 to 16.
9. A modified carrier protein according to paragraph 8, wherein the modified carrier protein comprises or consists of an amino acid sequence at least 80%, 85%, 90%, 92%, 95%, 96% or 97% identical to any one of SEQ ID NOs. 1 to 16.
10. The modified carrier protein of any one of paragraphs 1-9, wherein the modified carrier protein is glycosylated.
11. A conjugate (e.g. bioconjugate) comprising a modified carrier protein of any one of paragraphs 1-10, wherein the modified carrier protein is linked to a polysaccharide.
12. The conjugate (e.g. bioconjugate) of paragraph 11, wherein the polysccharide is linked to an amino acid on the modified carrier protein selected from asparagine, aspartic acid, glutamic acid, lysine, cysteine, tyrosine, histidine, arginine or tryptophan (e.g. asparagine).
13. The conjugate (e.g. bioconjugate) of paragraph 11 or paragraph 12, wherein the polysaccharide is a bacterial capsular polysaccharide
14. The conjugate (e.g. bioconjugate) of paragraph 13, wherein the capsular polysaccharide is selected from the group consisting of: Staphylococcus aureus type 5 capsular saccharide, Staphylococcus aureus type 8 capsular saccharide, N. meningitidis serogroup A capsular saccharide (MenA), N. meningitidis serogroup C capsular saccharide (MenC), N. meningitidis serogroup Y capsular saccharide (MenY), N. meningitidis serogroup W capsular saccharide (MenW), H. influenzae type b capsular saccharide (Hib), Group B Streptococcus group I capsular saccharide, Group B Streptococcus group II capsular saccharide, Group B Streptococcus group III capsular saccharide, Group B Streptococcus group IV capsular saccharide, Group B Streptococcus group V capsular saccharide, Vi saccharide from Salmonella typhi, N. meningitidis LPS (such as L3 and/or L2), M. catarrhalis LPS, H. influenzae LPS, Shigella O-antigens, P. aeruginosa O-antigens, E. coli O-antigens or S. pneumoniae capsular polysaccharide.
15. The conjugate (e.g. bioconjugate) of paragraph 13 or paragraph 14, wherein the capsular polysaccharide is from the same organism as the carrier protein.
16. The conjugate (e.g. bioconjugate) of paragraph 15, wherein the capsular polysaccharide is from a different organism to the carrier protein.
17. A polynucleotide encoding the modified carrier protein of any one of paragraphs 1-10.
18. A vector comprising the polynucleotide of paragraph 17.
19. A host cell comprising:
   a. one or more nucleic acids that encode glycosyltransferase(s);
   b. a nucleic acid that encodes an oligosaccharyl transferase;
   c. a nucleic acid that encodes a modified carrier protein according to any one of paragraphs 1-10; and optionally
   d. a nucleic acid that encodes a polymerase (e.g. wzy).
20. The host cell of paragraph 19, wherein said host cell comprises (a) a glycosyltransferase that assembles a hexose monosaccharide derivative onto undecaprenyl pyrophosphate (Und-PP) and (b) one or more glycosyltransferases capable of adding a monosaccharide to the hexose monosaccharide derivative assembled on Und-PP.
21. The host cell of paragraph 20, wherein said glycosyltransferase that assembles a hexose monosaccharide derivative onto Und-PP is heterologous to the host cell and/or heterologous to one or more of the genes that encode glycosyltransferase(s) optionally wherein said glycosyltransferase that assembles a hexose monosaccharide derivative onto Und-PP is from Escherichia species, Shigella species, Klebsiella species, Xhantomonas species, Salmonella species, Yersinia species, Aeromonas species, Francisella species, Helicobacter species, Proteus species, Lactococcus species, Lactobacillus species, Pseudomonas species, Corynebacterium species, Streptomyces species, Streptococcus species, Enterococcus species, Staphylococcus species, Bacillus species, Clostridium species, Listeria species, or Campylobacter species, optionally wecA (e.g. wecA from E. coli).
22. The host cell of any one of paragraphs 19-21, wherein said hexose monosaccharide derivative is any monosaccharide in which C-2 position is modified with an acetamido group such as N-acetylglucosamine (GlcNAc), N-acetylgalactoseamine (GalNAc), 2,4-Diacetamido-2,4,6-trideoxyhexose (DATDH). N-acetylfucoseamine (FucNAc), or N-acetylquinovosamine (QuiNAc).
23. The host cell of any one of paragraphs 19-22, wherein said one or more glycosyltransferases capable of adding a monosaccharide to the hexose monosaccharide derivative assembled on Und-PP is the galactofuranosyltransferase (wbeY) from E. coli O28 or the galactofuranosyltransferase (wfdK) from E. coli O167 or are the galactofuranosyltransferase (wbeY) from E. coli O28 and the galactofuranosyltransferase (wfdK) from E. coli O167.
24. The host cell of any one of paragraphs 19-23, wherein the glycosyltransferases comprise a glycosyltransferase that is capable of adding the hexose monosaccharide present at the reducing end of the first repeat unit of the donor oligosaccharide or polysaccharide to the hexose monosaccharide derivative, optionally wherein said one or more glycosyltransferases capable of adding a monosaccharide to the hexose monosaccharide derivative comprise galactosyltransferase (wciP), optionally from E. coli O21, and optionally comprising a glycosyltransferase that is capable of adding the monosaccharide that is adjacent to the hexose monosaccharide present at the reducing end of the first repeat unit of the donor oligosaccharide or polysaccharide to the hexose monosaccharide present at the reducing end of the first repeat unit of the donor oligosaccharide or polysaccharide, optionally glucosyltransferase (wciQ), optionally from E. coli O21.
25. The host cell of any one of paragraphs 19-24 wherein the oligosaccharyl transferase is derived from Campylobacter jejuni, optionally wherein said oligosaccharyl transferase is pglB of C. jejuni, optionally wherein the pglB gene of C. jejuni is integrated into the host cell genome and optionally wherein at least one gene of the host cell has been functionally inactivated or deleted, optionally wherein the waaL gene of the host cell has been functionally inactivated or deleted, optionally wherein the waaL gene of the host cell has been replaced by a nucleic acid encoding an oligosaccharyltransferase, optionally wherein the waaL gene of the host cell has been replaced by C. jejuni pglB.
26. The host cell of any one of paragraphs 19-25, wherein the nucleic acid that encodes the modified carrier protein is in a plasmid in the host cell.
27. The host cell of any one of paragraphs 19-26, wherein the nucleic acid that encodes the modified carrier protein is integrated into the genome of the host cell.
28. The host cell of any one of paragraphs 19-27, wherein the host cell is E. coli.
29. A method of producing a bioconjugate that comprises a modified carrier protein linked to a saccharide, said method comprising (i) culturing the host cell of any one of paragraphs 19-28 under conditions suitable for the production of proteins and (ii) isolating the bioconjugate.
30. A bioconjugate produced by the process of paragraph 29, wherein said bioconjugate comprises a polysaccharide linked to a modified carrier protein.
31. An immunogenic composition comprising the modified carrier protein of any one of paragraphs 1-10, or the conjugate or the bioconjugate of any one of paragraphs 11-16.
32. A method of making the immunogenic composition of paragraph 31 comprising the step of mixing the modified carrier protein or the conjugate or the bioconjugate with a pharmaceutically acceptable excipient or carrier.
33. A vaccine comprising the immunogenic composition of paragraph 31 and a pharmaceutically acceptable excipient or carrier.
34. A method for the treatment or prevention of a bacterial infection in a subject in need thereof comprising administering to said subject a therapeutically effective amount of the modified carrier protein of any one of paragraphs 1-10, or the conjugate or the bioconjugate of any one of paragraphs 11-16.
35. A method of immunising a human host against a bacterial infection comprising administering to the host an immunoprotective dose of the modified carrier protein of any one of paragraphs 1-10, or the conjugate or the bioconjugate of any one of paragraphs 11-16.
36. A method of inducing an immune response to a bacterium in a subject, the method comprising administering to a subject a therapeutically or prophylactically effective amount of the modified carrier protein any one of paragraphs 1-10, or the conjugate or the bioconjugate of any one of paragraphs 11-16.
37. A modified carrier protein of any one of paragraphs 1-10, or the conjugate or the bioconjugate of any one of paragraphs 11-16, for use in the treatment or prevention of a disease caused by bacterial infection.
38. Use of the modified carrier protein of any one of paragraphs 1-10, or the conjugate or the bioconjugate of any one of paragraphs 11-16 in the manufacture of a medicament for the treatment or prevention of a disease caused by bacterial infection.
39. The method of any one of paragraphs 34-36, or a carrier protein, conjugate or bioconjugate for use of paragraph 37, or the use of paragraph 38, wherein said bacterium or bacterial infection is selected from the group consisting of Staphylococcus aureus, N. meningitidis, H. influenzae, H. influenzae type b, Group B Streptococcus, S. typhi, M. catarrhalis, S. flexneri, P. aeruginosa, E. coli or S. pneumoniae.
40. A method of measuring the level of glycosylation site occupancy of a carrier protein according to any one of paragraphs 1 to 10, said method comprising: digesting the glycosylated carrier protein with a protease; subjecting the digested protein to LC-MS; determining the concentration U of unmodified carrier protein; determining the concentration T of total carrier protein; and calculating glycosylation site occupancy according to the following equation:

$$Site Occupancy ( \% ) = \frac{( Total - unmodified ) carrier concentration}{Total carrier concentration} \times 100$$

41. A method according to paragraph 40, wherein the concentration U of unmodified carrier protein is determined by determining the concentration of a peptide fragment corresponding to a consensus sequence.
42. A method according to paragraph 40 or paragraph 41, wherein the concentration T of total carrier protein is determined by determining the concentration of one or more peptide fragments which are unique to said carrier protein.
43. A method according to any one of paragraphs 40 to 42, wherein the protease is trypsin.

### SEQUENCE LISTINGS

SEQ ID NO: 1
Amino acid sequence of mature wild-type EPA.
Bold and underlined are the residues
substituted/removed for detoxification.
Organism: Pseudomonas aeruginosa.
AEEAFDLWNECAKACVLDLKDGVRSSRMSVDPAIA
DTNGQGVLHYSMVLEGGNDALKLAIDNALSITSDG
LTIRLEGGVEPNKPVRYSYTRQARGSWSLNWLVPI
GHEKPSNIKVFIHELNAGNQLSHMSPIYTIEMGDE
LLAKLARDATFFVRAHESNEMQPTLAISHAGVSVV
MAQAQPRREKRWSEWASGKVLCLLDPLDGVYNYLA
QQRCNLDDTWEGKIYRVLAGNPAKHDLDIKPTVIS
HRLHFPEGGSLAALTAHQACHLPLEAFTRHRQPRG
WEQLEQCGYPVQRLVALYLAARLSWNQVDQVIRNA
LASPGSGGDLGEAIREQPEQARLALTLAAAESERF
VRQGTGNDEAGAASADVVSLTCPVAAGECAGPADS
GDALLERNYPTGAEFLGDGGDVSFSTRGTQNWTVE
RLLQAHRQLEERGYVFVGYHGTFLEAAQSIVFGGV
RARSQDLDAIWRGFYIAGDPALAYGYAQDQEPDAR
GRIRNGALLRVYVPRWSLPGFYRTGLTLAAPEAAG
EVERLIGHPLPLRLDAITGPEEEGGR [symbol] TILGWPL
AERTVVIPSAIPTDPRNVGGDLDPSSIPDKEQAIS
ALPDYASQPGKPPREDLK
SEQ ID NO: 2
Amino acid sequence of EPA with L552V/AE553
detoxifying mutation (bold, underlined).
Artificial sequence.
AEEAFDLWNECAKACVLDLKDGVRSSRMSVDPAIA
DTNGQGVLHYSMVLEGGNDALKLAIDNALSITSDG
LTIRLEGGVEPNKPVRYSYTRQARGSWSLNWLVPI
GHEKPSNIKVFIHELNAGNQLSHMSPIYTIEMGDE
LLAKLARDATFFVRAHESNEMQPTLAISHAGVSVV
MAQAQPRREKRWSEWASGKVLCLLDPLDGVYNYLA
QQRCNLDDTWEGKIYRVLAGNPAKHDLDIKPTVIS
HRLHFPEGGSLAALTAHQACHLPLEAFTRHRQPRG
WEQLEQCGYPVQRLVALYLAARLSWNQVDQVIRNA
LASPGSGGDLGEAIREQPEQARLALTLAAAESERF
VRQGTGNDEAGAASADVVSLTCPVAAGECAGPADS
GDALLERNYPTGAEFLGDGGDVSFSTRGTQNWTVE
RLLQAHRQLEERGYVFVGYHGTFLEAAQSIVFGGV
RARSQDLDAIWRGFYIAGDPALAYGYAQDQEPDAR
GRIRNGALLRVYVPRWSLPGFYRTGLTLAAPEAAG
EVERLIGHPLPLRLDAITGPEEEGGRVTILGWPLA
ERTWIPSAIPTDPRNVGGDLDPSSIPDKEQAISAL
PDYASQPGKPPREDLK
SEQ ID NO: 3:
Tetanus toxin precursor TT (AA 1-1315)
without initial methionine.
Organism: Clostridium tetani
MPITINNFRYSDPVNNDTIIMMEPPYCKGLDIYYK
AFKITDRIWIVPERYEFGTKPEDFNPPSSLIEGAS
EYYDPNYLRTDSDKDRFLQTMVKLFNRIKNNVAGE
ALLDKIINAIPYLGNSYSLLDKFDTNSNSVSFNLL
EQDPSGATTKSAMLTNLIIFGPGPVLNKNEVRGIV
LRVDNKNYFPCRDGFGSIMQMAFCPEYVPTFDNVI
ENITSLTIGKSKYFQDPALLLMHELIHVLHGLYGM
QVSSHEIIPSKQEIYMQHTYPISAEELFTFGGQDA
NLISIDIKNDLYEKTLNDYKAIANKLSQVTSCNDP
NIDIDSYKQIYQQKYQFDKDSNGQYIVNEDKFQIL
YNSIMYGFTEIELGKKFNIKTRLSYFSMNHDPVKI
PNLLDDTIYNDTEGFNIESKDLKSEYKGQNMRVNT
NAFRNVDGSGLVSKLIGLCKKIIPPTNIRENLYNR
TASLTDLGGELCIKIKNEDLTFIAEKNSFSEEPFQ
DEIVSYNTKNKPLNFNYSLDKIIVDYNLQSKITLP
NDRTTPVTKGIPYAPEYKSNAASTIEIHNIDDNTI
YQYLYAQKSPTTLQRITMTNSVDDALINSTKIYSY
FPSVISKVNQGAQGILFLQWVRDIIDDFTNESSQK
TTIDKISDVSTIVPYIGPALNIVKQGYEGNFIGAL
ETTGVVLLLEYIPEITLPVIAALSIAESSTQKEKI
IKTIDNFLEKRYEKWIEVYKLVKAKWLGTVNTQFQ
KRSYQMYRSLEYQVDAIKKIIDYEYKIYSGPDKEQ
IADEINNLKNKLEEKANKAMININIFMRESSRSFL
VNQMINEAKKQLLEFDTQSKNILMQYIKANSKFIG
ITELKKLESKINKVFSTPIPFSYSKNLDCWVDNEE
DIDVILKKSTILNLDINNDIISDISGFNSSVITYP
DAQLVPGINGKAIHLVNNESSEVIVHKAMDIEYND
MFNNFTVSFWLRVPKVSASHLEQYGTNEYSIISSM
KKHSLSIGSGWSVSLKGNNLIWTLKDSAGEVRQIT
FRDLPDKFNAYLANKWVFITITNDRLSSANLYING
VLMGSAEITGLGAIREDNNITLKLDRCNNNNQYVS
IDKFRIFCKALNPKEIEKLYTSYLSITFLRDFWGN
PLRYDTEYYLIPVASSSKDVQLKNITDYMYLTNAP
SYTNGKLNIYYRRLYNGLKFIIKRYTPNNEIDSFV
KSGDFIKLYVSYNNNEHIVGYPKDGNAFNNLDRIL
RVGYNAPGIPLYKKMEAVKLRDLKTYSVQLKLYDD
KNASLGLVGTHNGQIGNDPNRDILIASNWYFNHLK
DKILGCDWYFVPTDEGWTND
SEQ ID NO: 4 Diphtheria toxin (DT).
Organism: Corynebacterium diphtheriai
GADDVVDSSKSFVMENFSSYHGTKPGYVDSIQKGI
QKPKSGTQGNYDDDWKGFYSTDNKYDAAGYSVDNE
NPLSGKAGGVVKVTYPGLTKVLALKVDNAETIKKE
LGLSLTEPLMEQVGTEEFIKRFGDGASRVVLSLPF
AEGSSSVEYINNWEQAKALSVELEINFETRGKRGQ
DAMYEYMAQACAGNRVRRSVGSSLSCINLDWDVIR
DKTKTKIESLKEHGPIKNKMSESPNKTVSEEKAKQ
YLEEFHQTALEHPELSELKTVTGTNPVFAGANYAA
WAVNVAQVIDSETADNLEKTTAALSILPGIGSVMG
IADGAVHHNTEEIVAQSIALSSLMVAQAIPLVGEL
VDIGFAAYNFVESIINLFQVVHNSYNRPAYSPGHK
TQPFLHDGYAVSWNTVEDSIIRTGFQGESGHDIKI
TAENTPLPIAGVLLPTIPGKLDVNKSKTHISVNGR
KIRMRCRAIDGDVTFCRPKSPVYVGNGVHANLHVA
FHRSSSEKIHSNEISSDSIGVLGYQKTVDHTKVNS
KLSLFFEIKS
SEQ ID NO: 5:
CRM197, non-toxic mutant of diphtheria toxin.
Artificial sequence.
GADDVVDSSKSFVMENFSSYHGTKPGYVDSIQKGI
QKPKSGTQGNYDDDWKEFYSTDNKYDAAGYSVDNE
NPLSGKAGGVVKVTYPGLTKVLALKVDNAETIKKE
LGLSLTEPLMEQVGTEEFIKRFGDGASRVVLSLPF
AEGSSSVEYINNWEQAKALSVELEINFETRGKRGQ
DAMYEYMAQACAGNRVRRSVGSSLSCINLDWDVIR
DKTKTKIESLKEHGPIKNKMSESPNKTVSEEKAKQ
YLEEFHQTALEHPELSELKTVTGTNPVFAGANYAA
WAVNVAQVIDSETADNLEKTTAALSILPGIGSVMG
IADGAVHHNTEEIVAQSIALSSLMVAQAIPLVGEL
VDIGFAAYNFVESIINLFQVVHNSYNRPAYSPGHK
TQPFLHDGYAVSWNTVEDSIIRTGFQGESGHDIKI
TAENTPLPIAGVLLPTIPGKLDVNKSKTHISVNGR
KIRMRCRAIDGDVTFCRPKSPVYVGNGVHANLHVA
FHRSSSEKIHSNEISSDSIGVLGYQKTVDHTKVNS
KLSLFFEIKS
SEQ ID NO: 6:
Hcp1. Organism: Pseudomonas aeruginosa
MAVDMFIKIGDVKGESKDKTHAEEIDVLAWSWGMS
QSGSMHMGGGGGAGKVNVQDLSFTKYIDKSTPNLM
MACSSGKHYPQAKLTIRKAGGENQVEYLIITLKEV
LVSSVSTGGSGGEDRLTENVTLNFAQVQVDYQPQK
ADGAKDGGPIKYGWNIRQNVQA
SEQ ID NO: 7:
PspA, phage shock protein A without initial
methionine. Organism: Escherichia coli
GIFSRFADIVNANINALLEKAEDPQKLVRLMIQEM
EDTLVEVRSTSARALAEKKQLTRRIEQASAREVEW
QEKAELALLKEREDLARAALIEKQKLTDLIKSLEH
EVTLVDDTLARMKKEIGELENKLSETRARQQALML
RHQAANSSRDVRRQLDSGKLDEAMARFESFERRID
QMEAEAESHSFGKQKSLDDQFAELKADDAISEQLA
QLKAKMKQDNQ
SEQ ID NO: 8:
MBP, Maltose/maltodextrin binding protein.
Organism: Escherichia coli
MKIKTGARILALSALTTMMFSASALAKIEEGKLVI
WINGDKGYNGLAEVGKKFEKDTGIKVTVEHPDKLE
EKFPQVAATGDGPDIIFWAHDRFGGYAQSGLLAEI
TPDKAFQDKLYPFTWDAVRYNGKLIAYPIAVEALS
LIYNKDLLPNPPKTWEEIPALDKELKAKGKSALMF
NLQEPYFTWPLIAADGGYAFKYENGKYDIKDVGVD
NAGAKAGLTFLVDLIKNKHMNADTDYSIAEAAFNK
GETAMTINGPWAWSNIDTSKVNYGVTVLPTFKGQP
SKPFVGVLSAGINAASPNKELAKEFLENYLLTDEG
LEAVNKDKPLGAVALKSYEEELAKDPRIAATMENA
QKGEIMPNIPQMSAFWYAVRTAVINAASGRQTVDE
ALKDAQTRITK
SEQ ID NO: 9:
mature mtrE, Membrane Transporter E.
Organism: Neisseria gonorrhoeae
MIPQYEQPKVEVAETFQNDTSVSSIRAVDLGWHDY
FADPRLQKLIDIALERNTSLRTAVLNSEIYRKQYM
IERNNLLPTLAANANGSRQGSLSGGNVSSSYNVGL
GAASYELDLFGRVRSSSEAALQGYFASVANRDAAH
LSLIATVAKAYFNERYAEEAMSLAQRVLKTREETY
NAVRIAVQGRRDFRRRPAPAEALIESAKADYAHAA
RSREQARNALATLINRPIPEDLPAGLPLDKQFFVE
KLPAGLSSEVLLDRPDIRAAEHALKQANANIGAAR
AAFFPSIRLTGSVGTGSVELGGLFKSGTGVWAFAP
SITLPIFTWGTNKANLDVAKLRQQAQIVAYESAVQ
SAFQDVANALAAREQLDKAYDALSKQSRASKEALR
LVGLRYKHGVSGALDLLDAERSSYSAEGAALSAQL
TRAENLADLYKALGGGLKRDTQTGK
SEQ ID NO: 10-
Wild-type mature ClfANI N2N3.
Organism: Staphylococcus aureus.
ASENSVTQSDSASNESKSNDSSSVSAAPKTDDTNV
SDTKTSSNTNNGETSVAQNPAQQETTQSSSTNATT
EETPVTGEATTTTTNQANTPATTQSSNTNAEELVN
QTSNETTFNDTNTVSSVNSPQNSTNAENVSTTQDT
STEATPSNNESAPQSTDASNKDVVNQAVNTSAPRM
RAFSLAAVAADAPAAGTDITNQLTNVTVGIDSGTT
VYPHQAGYVKLNYGFSVPNSAVKGDTFKITVPKEL
NLNGVTSTAKVPPIMAGDQVLANGVIDSDGNVIYT
FTDYVNTKDDVKATLTMPAYIDPENVKKTGNVTLA
TGIGSTTANKTVLVDYEKYGKFYNLSIKGTIDQID
KTNNTYRQTIYVNPSGDNVIAPVLTGNLKPNTDSN
ALIDQQNTSIKVYKVDNAADLSESYFVNPENFEDV
TNSVNITFPNPNQYKVEFNTPDDQITTPYIVVVNG
HIDPNSKGDLALRSTLYGYNSNIIWRSMSWDNEVA
FNNGSGSGDGIDKPWPEQPDEPGEIEPIPED
SEQ ID NO: 11-Wild-
type mature ClfAN2N3.
Organism: Staphylococcus aureus.
VAADAPAAGTDITNQLTNVTVGIDSGTTVYPHQAG
YVKLNYGFSVPNSAVKGDTFKITVPKELNLNGVTS
TAKVPPIMAGDQVLANGVIDSDGNVIYTFTDYVNT
KDDVKATLTMPAYIDPENVKKTGNVTLATGIGSTT
ANKTVLVDYEKYGKFYNLSIKGTIDQIDKTNNTYR
QTIYVNPSGDNVIAPVLTGNLKPNTDSNALIDQQN
TSIKVYKVDNAADLSESYFVNPENFEDVTNSVNIT
FPNPNQYKVEFNTPDDQITTPYIVVVNGHIDPNSK
GDLALRSTLYGYNSNIIWRSMSWDNEVAFNNGSGS
GDGIDKPWPEQPDEPGEIEPIPED
SEQ ID NO: 12-ClfAN2N3P116S
/Y118A. Artificial sequence.
VAADAPAAGTDITNQLTNVTVGIDSGTTVYPHQAG
YVKLNYGFSVPNSAVKGDTFKITVPKELNLNGVTS
TAKVPPIMAGDQVLANGVIDSDGNVIYTFTDYVNT
KDDVKATLTMSAAIDPENVKKTGNVTLATGIGSTT
ANKTVLVDYEKYGKFYNLSIKGTIDQIDKTNNTYR
QTIYVNPSGDNVIAPVLTGNLKPNTDSNALIDQQN
TSIKVYKVDNAADLSESYFVNPENFEDVTNSVNIT
FPNPNQYKVEFNTPDDQITTPYIVVVNGHIDPNSK
GDLALRSTLYGYNSNIIWRSMSWDNEVAFNNGSGS
GDGIDKPWPEQPDEPGEIEP [symbol] PED
SEQ ID NO: 13:
Amino acid sequence of mature wild-type Hla.
Organism: Staphylococcus aureus.
ADSDINIKTGTTDIGSNTTVKTGDLVTYDKENGMH
KKVFYSFIDDKNHNKKLLVIRTKGTIAGQYRVYSE
EGANKSGLAWPSAFKVQLQLPDNEVAQISDYYPRN
SIDTKEYMSTLTYGFNGNVTGDDTGKIGGLIGANV
SIGHTLKYVQPDFKTILESPTDKKVGWKVIFNNMV
NQNWGPYDRDSWNPVYGNQLFMKTRNGSMKAADNF
LDPNKASSLLSSGFSPDFATVITMDRKASKQQTNI
DVIYERVRDDYQLHWTSTNWKGTNTKDKWIDRSSE
RYKIDWEKEEMTN
SEQ ID NO: 14-Amino acid
sequence of mature HlaH35L.
Artificial sequence.
ADSDINIKTGTTDIGSNTTVKTGDLVTYDKENGML
KKVFYSFIDDKNHNKKLLVIRTKGTIAGQYRVYSE
EGANKSGLAWPSAFKVQLQLPDNEVAQISDYYPRN
SIDTKEYMSTLTYGFNGNVTGDDTGKIGGLIGANV
SIGHTLKYVQPDFKTILESPTDKKVGWKVIFNNMV
NQNWGPYDRDSWNPVYGNQLFMKTRNGSMKAADNF
LDPNKASSLLSSGFSPDFATVITMDRKASKQQTNI
DVIYERVRDDYQLHWTSTNWKGTNTKDKWIDRSSE
RYKIDWEKEEMTN
SEQ ID NO: 15-Amino acid
sequence of mature Hla H35L/
H48C/G122C, Artificial sequence.
ADSDINIKTGTTDIGSNTTVKTGDLVTYDKENGML
KKVFYSFIDDKNCNKKLLVIRTKGTIAGQYRVYSE
EGANKSGLAWPSAFKVQLQLPDNEVAQISDYYPRN
SIDTKEYMSTLTYGFNCNVTGDDTGKIGGLIGANV
SIGHTLKYVQPDFKTILESPTDKKVGWKVIFNNMV
NQNWGPYDRDSWNPVYGNQLFMKTRNGSMKAADNF
LDPNKASSLLSSGFSPDFATVITMDRKASKQQTNI
DVIYERVRDDYQLHWTSTNWKGTNTKDKWIDRSSE
RYKIDWEKEEMTN
SEQ ID NO: 16:
HlaPSGS. Artificial sequence.
ADSDINIKTGTTDIGSNTTVKTGDLVTYDKENGMH
KKVFYSFIDDKNHNKKLLVIRTKGTIAGQYRVYSE
EGANKSGLAWPSAFKVQLQLPDNEVAQISDYYPRN
SIDTPSGSVQPDFKTILESPTDKKVGWKVIFNNMV
NQNWGPYDRDSWNPVYGNQLFMKTRNGSMKAADNF
LDPNKASSLLSSGFSPDFATVITMDRKASKQQTNI
DVIYERVRDDYQLHWTSTNWKGTNTKDKWIDRSSE
RYKIDWEKEEMTN
SEQ ID NO: 17:
Minimal PgIB glycosite consensus sequence.
Artificial sequence.
D/E-X₁-N-X₂-S/T
wherein X₁ and X₂ are any amino acid apart
from proline.
SEQ ID NO : 18:
Full PgIB glycosite consensus sequence.
Artificial sequence
K-D/E-X₁-N-X₂-S/T-K
wherein X₁ and X₂ are any amino acid
apart from proline.
SEQ ID NO: 19:
MS quantification-compatible PgIB glycosite
consensus sequence. Artificial sequence.
K/R-D/E-X₁-N-X₂-S/T-Z₁-Z₂-R/K
wherein X₁ and X₂ are any amino acid
apart from proline, and Z₁ and Z₂. are not
lysine or arginine.
SEQ ID NO: 20:
MS quantification-compatible PgIB glycosite
consensus sequence. Artificial sequence.
K-D/E-X₁-N-X₂-S/T-S-A-R
wherein X₁ and X₂ are any amino acid
apart from proline.
SEQ ID NO: 21-Flgl
signal sequence.
Organism: Shigella flexneri
MI K FL SALILLLVTTAAQA
SEQ ID NO: 22-OmpA
signal sequence.
Organism: Escherichia coli
MKKTAIAIAVALAGFATVAQA
SEQ ID NO: 23-MalE
signal sequence.
Organism: Escherichia coli
MKIKTGARILALSALTTMMFSASALA
SEQ ID NO: 24
PelB signal sequence.
Organism: Pectobacterium carotovorum
(Erwinia carotovora).
MKYLLPTAAAGLLLLAAQPAMA
SEQ ID NO: 25
LTIIb signal sequence.
Organism: Escherichia coli
MSFKKIIKAFVIMAALVSVQAHA
SEQ ID NO: 26
XynA signal sequence.
Organism: Bacillus subtilis
MFKFKKKFLVGLTAAFMSISMFSATASA
SEQ ID NO: 27
DsbA signal sequence.
Organism: Escherichia coli
MKKIWLALAGLVLAFSASA
SEQ ID NO: 28
TolB signal sequence.
Organism: Escherichia coli
MKQAL RVAFGFLILWASVLHA
SEQ ID NO: 29
SipA signal sequence.
Organism: Streptococcus agalactiae
MKMNKKVLLTSTMAASLLSVASVQAS
SEQ ID NO: 30:
Amino acid sequence of Hla H35L/H48C/G122C
with N-terminal S, Flgl signal sequence,
C-terminal GSHRHR, and KDQNRTK
substitution for residue K131.
Artificial sequence.
MIKFLSALILLLVTTAAQASADSDINIKTGTTDIG
SNTTVKTGDLVTYDKENGMLKKVFYSFIDDKNCNK
KLLVIRTKGTIAGQYRVYSEEGANKSGLAWPSAFK
VQLQLPDNEVAQISDYYPRNSIDTKEYMSTLTYGF
NCNVTGDDTGKDQNRTKIGGLIGANVSIGHTLKYV
QPDFKTILESPTDKKVGWKVIFNNMVNQNWGPYDR
DSWNPVYGNQLFMKTRNGSMKAADNFLDPNKASSL
LSSGFSPDFATVITMDRKASKQQTNIDVIYERVRD
DYQLHWTSTNWKGTNTKDKWIDRSSERYKIDWEKE
EMTNGSHRHR
SEQ ID NO: 31:
Amino acid sequence of mature Hla
H35L/G122C/H48C with N-terminal S,
Flgl signal sequence, C-terminal
GSHRHR and KDSNITSAR substitution
for residue K131, Artificial sequence.
MIKFLSALILLLVTTAAQASADSDINIKTGTTDIG
SNTTVKTGDLVTYDKENGMLKKVFYSFIDDKNCNK
KLLVIRTKGTIAGQYRVYSEEGANKSGLAWPSAFK
VQLQLPDNEVAQISDYYPRNSIDTKEYMSTLTYGF
NCNVTGDDTGKDSNITSARIGGLIGANVSIGHTLK
YVQPDFKTILESPTDKKVGWKVIFNNMVNQNWGPY
DRDSWNPVYGNQLFMKTRNGSMKAADNFLDPNKAS
SLLSSGFSPDFATVITMDRKASKQQTNIDVIYERV
RDDYQLHWTSTNWKGTNTKDKWIDRSSERYKIDWE
KEEMTNGSHRHR
SEQ ID NO: 32:
Amino acid sequence of mature Hla H35L/G122C/
H48C with N-terminal S, Flgl signal
sequence, C-terminal GSHRHR and KDSNSTSAR
substitution for residue K131, Artificial
sequence.
MIKFLSALILLLVTTAAQASADSDINIKTGTTDIG
SNTTVKTGDLVTYDKENGMLKKVFYSFIDDKNCNK
KLLVIRTKGTIAGQYRVYSEEGANKSGLAWPSAFK
VQLQLPDNEVAQISDYYPRNSIDTKEYMSTLTYGF
NCNVTGDDTGKDSNSTSARIGGLIGANVSIGHTLK
YVQPDFKTILESPTDKKVGWKVIFNNMVNQNWGPY
DRDSWNPVYGNQLFMKTRNGSMKAADNFLDPNKAS
SLLSSGFSPDFATVITMDRKASKQQTNIDVIYERV
RDDYQLHWTSTNWKGTNTKDKWIDRSSERYKIDWE
KEEMTNGSHRHR
SEQ ID NO: 33:
Amino acid sequence of mature Hla H35L/G122C/
H48C with N-terminal S, Flgl signal sequence,
C-terminal GSHRHR and KDSNVTSAR substitution
for residue K131, Artificial sequence.
MIKFLSALILLLVTTAAQASADSDINIKTGTTDIG
SNTTVKTGDLVTYDKENGMLKKVFYSFIDDKNCNK
KLLVIRTKGTIAGQYRVYSEEGANKSGLAWPSAFK
VQLQLPDNEVAQISDYYPRNSIDTKEYMSTLTYGF
NCNVTGDDTGKDSNVTSARIGGLIGANVSIGHTLK
YVQPDFKTILESPTDKKVGWKVIFNNMVNQNWGPY
DRDSWNPVYGNQLFMKTRNGSMKAADNFLDPNKAS
SLLSSGFSPDFATVITMDRKASKQQTNIDVIYERV
RDDYQLHWTSTNWKGTNTKDKWIDRSSERYKIDWE
KEEMTNGSHRHR
SEQ ID NO: 34:
Amino acid sequence of mature Hla H35L/G122C/
H48C with N-terminal S, Flgl signal sequence,
C-terminal GSHRHR and KDSNATSAR substitution
for residue K131. Artificial sequence.
MIKFLSALILLLVTTAAQASADSDINIKTGTTDIG
SNTTVKTGDLVTYDKENGMLKKVFYSFIDDKNCNK
KLLVIRTKGTIAGQYRVYSEEGANKSGLAWPSAFK
VQLQLPDNEVAQISDYYPRNSIDTKEYMSTLTYGF
NCNVTGDDTGKDSNVTSARIGGLIGANVSIGHTLK
YVQPDFKTILESPTDKKVGWKVIFNNMVNQNWGPY
DRDSWNPVYGNQLFMKTRNGSMKAADNFLDPNKAS
SLLSSGFSPDFATVITMDRKASKQQTNIDVIYERV
RDDYQLHWTSTNWKGTNTKDKWIDRSSERYKIDWE
KEEMTNGSHRHR
SEQ ID NO: 35:
Amino acid sequence of mature Hla H35L/
H48C/G122C with KDQNRTK substitution
for residue K131. Artificial sequence.
ADSDINIKTGTTDIGSNTTVKTGDLVTYDKENGML
KKVFYSFIDDKNCNKKLLVIRTKGTIAGQYRVYSE
EGANKSGLAWPSAFKVQLQLPDNEVAQISDYYPRN
SIDTKEYMSTLTYGFNCNVTGDDTGKDQNRTKIGG
LIGANVSIGHTLKYVQPDFKTILESPTDKKVGWKV
IFNNMVNQNWGPYDRDSWNPVYGNQLFMKTRNGSM
KAADNFLDPNKASSLLSSGFSPDFATVITMDRKAS
KQQTNIDVIYERVRDDYQLHWTSTNWKGTNTKDKW
IDRSSERYKIDWEKEEMTN
SEQ ID NO: 36:
Amino acid sequence of mature Hla H35L/
G122C/H48C with KDSNITSAR substitution
for residue K131. Artificial sequence.
ADSDINIKTGTTDIGSNTTVKTGDLVTYDKENGML
KKVFYSFIDDKNCNKKLLVIRTKGTIAGQYRVYSE
EGANKSGLAWPSAFKVQLQLPDNEVAQISDYYPRN
SIDTKEYMSTLTYGFNCNVTGDDTGKDSNITSARI
GGLIGANVSIGHTLKYVQPDFKTILESPTDKKVGW
KVIFNNMVNQNWGPYDRDSWNPVYGNQLFMKTRNG
SMKAADNFLDPNKASSLLSSGFSPDFATVITMDRK
ASKQQTNIDVIYERVRDDYQLHWTSTNWKGTNTKD
KWIDRSSERYKIDWEKEEMTN
SEQ ID NO: 37:
Amino acid sequence of mature Hla H35L/G122C/
H48C with KDSNSTSAR substitution for residue
K131. Artificial sequence.
ADSDINIKTGTTDIGSNTTVKTGDLVTYDKENGML
KKVFYSFIDDKNCNKKLLVIRTKGTIAGQYRVYSE
EGANKSGLAWPSAFKVQLQLPDNEVAQISDYYPRN
SIDTKEYMSTLTYGFNCNVTGDDTGKDSNSTSARI
GGLIGANVSIGHTLKYVQPDFKTILESPTDKKVGW
KVIFNNMVNQNWGPYDRDSWNPVYGNQLFMKTRNG
SMKAADNFLDPNKASSLLSSGFSPDFATVITMDRK
ASKQQTNIDVIYERVRDDYQLHWTSTNWKGTNTKD
KWIDRSSERYKIDWEKEEMTN
SEQ ID NO: 38:
Amino acid sequence of mature Hla H35L/G122C/
H48C with KDSNVTSAR substitution for residue
K131. Artificial sequence.
ADSDINIKTGTTDIGSNTTVKTGDLVTYDKENGML
KKVFYSFIDDKNCNKKLLVIRTKGTIAGQYRVYSE
EGANKSGLAWPSAFKVQLQLPDNEVAQISDYYPRN
SIDTKEYMSTLTYGFNCNVTGDDTGKDSNVTSARI
GGLIGANVSIGHTLKYVQPDFKTILESPTDKKVGW
KVIFNNMVNQNWGPYDRDSWNPVYGNQLFMKTRNG
SMKAADNFLDPNKASSLLSSGFSPDFATVITMDRK
ASKQQTNIDVIYERVRDDYQLHWTSTNWKGTNTKD
KWIDRSSERYKIDWEKEEMTN
SEQ ID NO: 39:
Amino acid sequence of mature Hla H35L/G122C/
H48C with KDSNATSAR substitution for residue
K131. Artificial sequence.
ADSDINIKTGTTDIGSNTTVKTGDLVTYDKENGML
KKVFYSFIDDKNCNKKLLVIRTKGTIAGQYRVYSE
EGANKSGLAWPSAFKVQLQLPDNEVAQISDYYPRN
SIDTKEYMSTLTYGFNCNVTGDDTGKDSNVTSARI
GGLIGANVSIGHTLKYVQPDFKTILESPTDKKVGW
KVIFNNMVNQNWGPYDRDSWNPVYGNQLFMKTRNG
SMKAADNFLDPNKASSLLSSGFSPDFATVITMDRK
ASKQQTNIDVIYERVRDDYQLHWTSTNWKGTNTKD
KWIDRSSERYKIDWEKEEMTN
SEQ ID NO: 40:
KDQNRTK glycosite. Artificial sequence.
KDQNRTK
SEQ ID NO: 41:
KDQNATK glycosite. Artificial sequence.
KDQNATK
SEQ ID NO: 42:
KDSNITSAR glycosite. Artificial sequence.
KDSNITSAR
SEQ ID NO: 43:
KDSNSTSAR glycosite. Artificial sequence.
KDSNSTSAR
SEQ ID NO: 44:
KDSNVTSAR glycosite. Artificial sequence.
KDSNVTSAR
SEQ ID NO: 45:
KDSNATSAR glycosite. Artificial sequence.
KDSNATSAR
SEQ ID NO: 46:
MS quantification-compatible PgIB glycosite
consensus sequence. Artificial sequence.
K/R-Z₀₋₉-D/E-X-N-Y-S/T-Z₀₋₉-K/R
wherein X and Y are independently any amino
acid except proline, lysine or arginine, and
Z represents any amino acid except cysteine,
methionine, asparagine, glutamine, lysine or
arginine.
SEQ ID NO: 47:
MS quantification-compatible PgIB glycosite
consensus sequence. Artificial sequence.
K/R-Z₀₋₉-D/E-X-N-Y-S/T-Z₀₋₉-K/R
wherein X and Y are independently any amino
acid except proline, cysteine, methionine,
asparagine or glutamine, lysine or arginine,
and Z represents any amino acid except cysteine,
methionine, asparagine, glutamine, lysine
or arginine.
SEQ ID NO: 48:
Peptide 42T-50K named PTP-2.
Organism: Staphylococcus aureus.
TGDLVTYK
SEQ ID NO: 49:
Peptide 225A-234K named PTP-3.
Organism: Staphylococcus aureus.
AADNFLDPNK
SEQ. ID NO: 50: spacer
GSGGG
SEQ. ID NO: 51:
Amino acid sequence of mature Hla H35L/G122C/
H48C (starting with Ala-21, in bold) with
N-terminal S, KDSNITSAR glycosite substitution
for residue K131; glycosite KDSNVTSAR at N-
terminal with GSGGG spacers before and after
this glycosite; Flgl signal sequence; and
His tag at C-terminal. Artificial sequence.
MIKFLSALILLLVTTAAQASAGSGGGKDSNVTSAR
GSGGGKLADSDINIKTGTTDIGSNTTVKTGDLVTY
DKENGMLKKVFYSFIDDKNCNKKLLVIRTKGTIAG
QYRVYSEEGANKSGLAWPSAFKVQLQLPDNEVAQI
SDYYPRNSIDTKEYMSTLTYGFNCNVTGDDTGKDS
NITSARIGGLIGANVSIGHTLKYVQPDFKTILESP
TDKKVGWKVIFNNMVNQNWGPYDRDSWNPVYGNQL
FMKTRNGSMKAADNFLDPNKASSLLSSGFSPDFAT
VITMDRKASKQQTNIDVIYERVRDDYQLHWTSTNW
KGTNTKDKWIDRSSERYKIDWEKEEMTNGSHHHHH
H
SEQ ID NO: 52:
Amino acid sequence of mature Hla H35L/G122C/
H48C (starting with Ala-21, in bold)
with N-terminal S, KDSNITSAR glycosite
substitution for residue K131; glycosite
KDSNVTSAR at C-terminal with GSGGG spacers
before this glycosite; Flgl signal sequence;
and His tag at C-terminal. Artificial sequence.
MIKFLSALILLLVTTAAQASAADSDINIKTGTTDI
GSNTTVKTGDLVTYDKENGMLKKVFYSFIDDKNCN
KKLLVIRTKGTIAGQYRVYSEEGANKSGLAWPSAF
KVQLQLPDNEVAQISDYYPRNSIDTKEYMSTLTYG
FNCNVTGDDTGKDSNITSARIGGLIGANVSIGHTL
KYVQPDFKTILESPTDKKVGWKVIFNNMVNQNWGP
YDRDSWNPVYGNQLFMKTRNGSMKAADNFLDPNKA
SSLLSSGFSPDFATVITMDRKASKQQTNIDVIYER
VRDDYQLHWTSTNWKGTNTKDKWIDRSSERYKIDW
EKEEMTNLGSGGGKDSNVTSARGSHHHHHH
SEQ ID NO: 53:
Amino acid sequence of mature Hla H35L/G122C/
H48C (starting with Ala-21, in bold) with N-
terminal S, KDSNITSAR glycosite at C-terminal
end preceded by GSGGG spacers; glycosite
KDSNVTSAR at N-terminal with GSGGG spacers
before and after this glycosite; Flgl
signal sequence; and His tag at C-terminal.
Artificial sequence.
MIKFLSALILLLVTTAAQASAGSGGGKDSNVTSAR
GSGGGKLADSDINIKTGTTDIGSNTTVKTGDLVTY
DKENGMLKKVFYSFIDDKNCNKKLLVIRTKGTIAG
QYRVYSEEGANKSGLAWPSAFKVQLQLPDNEVAQI
SDYYPRNSIDTKEYMSTLTYGFNCNVTGDDTGIGG
LIGANVSIGHTLKYVQPDFKTILESPTDKKVGWKV
IFNNMVNQNWGPYDRDSWNPVYGNQLFMKTRNGSM
KAADNFLDPNKASSLLSSGFSPDFATVITMDRKAS
KQQTNIDVIYERVRDDYQLHWTSTNWKGTNTKDKW
IDRSSERYKIDWEKEEMTNLGSGGGKDSNITSARG
SHHHHHH

1. A modified carrier protein, modified in that it comprises one or more consensus sequence(s) comprising or consisting of the following amino acid sequence:
K/R-Z₀₋₉-D/E-X-N-Y-S/T-Z₀₋₉-K/R
wherein X and Y are independently any amino acid except proline, and Z represents any amino acid.

2. The modified carrier protein according to claim 1, wherein said consensus sequence is the amino acid sequence K/R-D/E-X₁-N-X₂-S/T-Z₁-Z₂-K/R (SEQ ID NO 19), wherein X₁ and X₂ are independently any amino acid except proline.

3. The modified carrier protein according to claim 1, wherein said consensus sequence comprises or consists of an amino acid sequence selected from the group consisting of SEQ ID NO: 20 and SEQ ID Nos: 42-45.

4. The modified carrier protein according to claim 1, wherein said consensus sequence (i) has been substituted for one or more amino acids of the carrier protein sequence, or (ii) has been inserted into the carrier protein sequence.

5. The modified carrier protein according to claim 1, comprising more than one said consensus sequence.

6. (canceled)

7. The modified carrier protein according to claim 1, wherein the carrier protein is CRM197, TT from Clostridium tetani, EPA from P. aeruginosa, Hcp1 from P. aeruginosa, Hla from S. aureus, ClfA from S. aureus, MBP from E. coli, PspA from E. coli, or MtrE from N. gonorrhoeae.

8. The modified carrier protein according to claim 7, wherein the carrier protein comprises or consists of an amino acid sequence of any one of SEQ ID Nos: 1 to 16 or an amino acid sequence at least 80%, 85%, 90%, 92%, 95%, 96%, 97%, 98% or 99% identical to any one of SEQ ID NOs. 1 to 16.

9-10. (canceled)

11. The conjugate comprising a modified carrier protein of claim 1, wherein the modified carrier protein is linked to a polysaccharide.

12. The conjugate of claim 11, wherein the polysaccharide is linked to an amino acid on the modified carrier protein selected from asparagine, aspartic acid, glutamic acid, lysine, cysteine, tyrosine, histidine, arginine or tryptophan.

13. The conjugate of claim 11, wherein the polysaccharide is a bacterial capsular polysaccharide.

14. The conjugate of claim 13, wherein the capsular polysaccharide is selected from the group consisting of: Staphylococcus aureus type 5 capsular saccharide, Staphylococcus aureus type 8 capsular saccharide, N. meningitidis serogroup A capsular saccharide (MenA), N. meningitidis serogroup C capsular saccharide (MenC), N. meningitidis serogroup Y capsular saccharide (MenY), N. meningitidis serogroup W capsular saccharide (MenW), H. influenzae type b capsular saccharide (Hib), Group B Streptococcus group I capsular saccharide, Group B Streptococcus group II capsular saccharide, Group B Streptococcus group III capsular saccharide, Group B Streptococcus group IV capsular saccharide, Group B Streptococcus group V capsular saccharide, Vi saccharide from Salmonella typhi, N. meningitidis LPS (such as L3 and/or L2), M. catarrhalis LPS, H. influenzae LPS, Shigella O-antigens, P. aeruginosa O-antigens, E. coli O-antigens or S. pneumoniae capsular polysaccharide.

15. The conjugate of claim 13, wherein the capsular polysaccharide is from the same organism as the carrier protein.

16. The conjugate of claim 11, which is a bioconjugate.

17. A polynucleotide encoding the modified carrier protein of claim 1.

18. A vector comprising the polynucleotide of claim 17.

19. A host cell comprising:
a. one or more nucleic acids that encode glycosyltransferase(s);
b. a nucleic acid that encodes an oligosaccharyl transferase;
c. a nucleic acid that encodes a modified carrier protein according to claim 1; and optionally
d. a nucleic acid that encodes a polymerase.

20-28. (canceled)

29. A method of producing a bioconjugate that comprises a modified carrier protein linked to a saccharide, said method comprising (i) culturing the host cell of claim 19 under conditions suitable for the production of proteins and (ii) isolating the bioconjugate.

30-31. (canceled)

32. An immunogenic composition comprising the modified carrier protein of claim 1.

33. (canceled)

34. A vaccine comprising the immunogenic composition of claim 32 and a pharmaceutically acceptable excipient or carrier.

35. A method for the treatment or prevention of a bacterial infection in a subject in need thereof comprising administering to said subject a therapeutically effective amount of the modified carrier protein of claim 1.

36-44. (canceled)
